SEQUENCE LISTING (10 sequences)

SEQ ID NO: 1 - protein, 10 aa, synthetic construct
HVLVMSATRR

SEQ ID NO: 2 - protein, 10 aa, synthetic construct
HVLVMSATKK

SEQ ID NO: 3 - protein, 12 aa, synthetic construct
HVLVMSATRRRR

SEQ ID NO: 4 - protein, 12 aa, synthetic construct
HVLVMSATKKKK

SEQ ID NO: 5 - protein, 8 aa, synthetic construct
HVLVMCAT

SEQ ID NO: 6 - protein, 23 aa, synthetic construct
AASSGVSTPGSAGHDIITEQPRS

SEQ ID NO: 7 - protein, 11 aa, synthetic construct
SNWKWWPGIFD

SEQ ID NO: 8 - protein, 24 aa, synthetic construct
GRKKRRQRRRPPQSSEIVLDGTDN

SEQ ID NO: 9 - protein, 8 aa, synthetic construct
HVLVMSAT

SEQ ID NO: 10 - protein, 5 aa, synthetic construct
SATRR

(19) United States
(12) Patent Application Publication
(10) Pub. No.: US 2024/0110004 A1
(43) Pub. Date: Apr. 4, 2024

(54) HUNTINGTIN MIMETIC PROTEIN-LIKE POLYMERS AND USES THEREOF

(71) Applicants: Northwestern University, Evanston, IL (US); The Regents of the University of California, Oakland, CA (US); Case Western Reserve University, Cleveland, OH (US)

(72) Inventors: Nathan C. GIANNESCHI, Wilmette, IL (US); Mara A. FATTAH, Evanston, IL (US); Wonmin CHOI, Evanston, IL (US); Xin QI, Cleveland, OH (US)

(21) Appl. No.: 18/452,059

(22) Filed: Aug. 18, 2023

Related U.S. Application Data
(60) Provisional application No. 63/399,064, filed on Aug. 18, 2022.

(51) Int. Cl. C08G 61/08; A61K 31/787; A61P 25/28

(52) U.S. Cl. CPC C08G 61/08; A61K 31/787; A61P 25/28; C08G 2261/122; C08G 2261/149; C08G 2261/228; C08G 2261/3342; C08G 2261/418

(57) ABSTRACT

Disclosed are protein-like polymers and uses thereof. The protein-like polymers generally comprise a polymer of formula (FX1) or (FX2). The polymer of formula (FX1) or (FX2) in some aspects inhibits the protein-protein interaction between VCP and mutant-type Huntingtin protein.

### CROSS-REFERENCE TO RELATED APPLICATIONS

This application claims the benefit of priority to U.S. Provisional Patent Application No. 63/399,064, filed Aug. 18, 2022, which is hereby incorporated by reference in its entirety.

### REFERENCE TO AN ELECTRONIC SEQUENCE LISTING

The content of the electronic sequence listing (339706_46-22_US_ST1.xml; Size: 9,604 bytes; and Date of Creation: Aug. 14, 2023) is herein incorporated by reference in its entirety.

### BACKGROUND

Huntington's disease (HD) is an autosomal dominant, fatal neurodegenerative disease affecting approximately 41,000 individuals in the United States with at least 200,000 other Americans with a 50% risk of developing the disease. Frank, S. Treatment of Huntington's Disease. Neurotherapeutics 2014, 11 (1), 153-160; Pan, L. et al. Huntington's Disease: New Frontiers in Therapeutics. Curr. Neurol. Neurosci. Rep. 2021, 21 (3); Gupta, S. et al. Demethyleneberberine: A Possible Treatment for Huntington's disease. 2021, 153 (May); Gibson, J. S. et al. Lifetime Neuropsychiatric Symptoms in Huntington's disease: Implications for Psychiatric Nursing. Arch. Psychiatr. Nurs. 2021, 35 (3), 284-289. HD is terminal upon diagnosis, as the expansion of the CAG codon from the Huntingtin (HTT) gene produces mutant huntingtin proteins and causes mitochondrial dysfunction. Mutant huntingtin protein (mtHtt) at the mitochondria binds to valosin containing protein (VCP) erroneously, marking the mitochondria for autophagy and resulting in neuronal cell death. Jimenez-Sanchez, M. et al. Huntington's Disease: Mechanisms of Pathogenesis and Therapeutic Strategies. Cold Spring Harb. Perspect. Med. 2017, 7 (7), 1-22; Franco-Iborra, S. et al. Mitochondrial Quality Control in Neurodegenerative Diseases: Focus on Parkinson's Disease and Huntington's Disease. Front. Neurosci. 2018, 12 (MAY), 1-25; Yin, X. et al. Mitochondria-Targeted Molecules MitoQ and SS31 Reduce Mutant Huntingtin-Induced Mitochondrial Toxicity and Synaptic Damage in Huntington's Disease. Hum. Mol. Genet. 2016, 25 (9), 1739-1753.

Although the wild type Huntingtin (Htt) gene has been identified and a genetic test is available to identify those individuals who carry the mutation and will succumb to the disease, there is currently no therapy to slow or prevent disease progression, only symptomatic treatments with limited impact. Recent developments of therapeutics targeting the striatum via injection are invasive, have low efficacy, and are not sustainable. Frank, S. Treatment of Huntington's Disease. Neurotherapeutics 2014, 11 (1), 153-160; Kordasiewicz, H. B. et al. Sustained Therapeutic Reversal of Huntington's Disease by Transient Repression of Huntingtin Synthesis. Neuron 2012, 74 (6), 1031-1044.

Peptides have been explored as biocompatible therapeutics with enhanced target-specificity for HD. Recently, the peptide sequence HVLVMCAT (HV3) was shown to target the VCP/mtHtt interaction. Guo, X. et al. VCP Recruitment to Mitochondria Causes Mitophagy Impairment and Neurodegeneration in Models of Huntington's Disease. Nat. Commun. 2016, 7. Lacking cell penetrative capability, this sequence was coupled to the cell penetrating, HIV-derived TAT peptide to yield the active, HV3-TAT peptide. HV3-TAT peptide blocks VCP translocation to mtHtt in the mitochondria, thereby rescuing neurons from mitochondrial destruction. Although this peptide displayed effective target engagement, it was limited by rapid enzymatic degradation and clearance. These limitations (poor pharmacokinetic properties and limited cell penetration) generally plague peptide-based therapeutics. In terms of pharmacokinetic limitations, a major factor is that peptides have an inherently low molecular weight, leading to rapid renal clearance. This rapid clearance is coupled with decreased stability and high susceptibility to swift degradation by proteolytic enzymes in vivo, leading to overall poor performance for peptide-based drugs. In their current state, free peptides, though advantageous for targeting and biocompatibility, generally present significant hurdles to translation to clinical use. Otvos, L.; Wade, J. D. Current Challenges in Peptide-Based Drug Discovery. Front. Chem. 2014, 2 (AUG), 8-11.

Thus, there is a demonstrated need for new, enabling platform technologies for treatment of neurodegenerative diseases or conditions. The invention provides such a platform. This and other advantages of the present invention will become apparent from the detailed description provided herein.

### SUMMARY OF THE INVENTION

In an aspect, the invention provides a polymer comprising a first repeating unit comprising a first polymer backbone group directly or indirectly covalently linked to a first functional sidechain comprising a peptide having 75% or greater sequence identity of an interaction site of VCP with mtHtt.

In an aspect, the invention provides a polymer comprising a first repeating unit comprising a first polymer backbone group directly or indirectly covalently linked to a first functional sidechain comprising at least a portion of a HV3 peptide.

In certain aspects, the invention provides a polymer further comprising a second repeating unit comprising a second polymer backbone group directly or indirectly covalently linked to a second functional sidechain comprising a therapeutic peptide that is different from the peptide of the first functional sidechain.

Further disclosed herein is a polymer characterized by a formula (FX1):

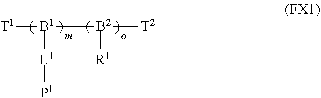

wherein each P¹ independently comprises a peptide; at least one P¹ independently, or in combination with other instances of P¹, comprises a HV3 peptide or a modified HV3 peptide; T¹ and T² are each independently polymer backbone terminating groups that can be the same or different; B¹ and B² are each independently a polymer backbone subunit; L¹ is optionally present and is a linking group; R¹ is independently a substituent; m is an integer from 2 to 1000; o is an integer from 0 to 1000; each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms; each instance of B¹, B², L¹, R¹, and P¹ is the same as or different from any other instance of B¹, B², L¹, R¹, and P¹, respectively; and when o is an integer from 1 to 1000 and/or at least one instance of P¹ is different from another instance of P¹, the polymer is a block copolymer or a statistical copolymer.

The present invention further includes a method for selectively inhibiting the mitochondrial autophagy pathway in a cell, the method comprising: contacting the cell with an effective amount of a polymer or a composition disclosed herein; wherein the contacting results in the selective inhibition of the mitochondrial autophagy pathway in the cell.

In some aspects, the invention provides a method for selectively inhibiting the interaction between VCP and mtHtt in a cell, the method comprising: contacting the cell with an effective amount of a polymer or a composition disclosed herein; wherein the contacting results in the selective inhibition of the interaction between VCP and mtHtt in the cell.

The present invention further includes a method for preventing or treating a neurodegenerative disease or condition in a subject, the method comprising: administering to the subject a therapeutically effective amount of a polymer or a composition disclosed herein; thereby preventing or treating the neurodegenerative disease or condition in the subject. In some aspects, the neurodegenerative disease or condition is Huntington's disease.

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

Unless otherwise indicated, graphs depicting statistical significance values with the following symbols are defined as: N.S. (p>0.05), * (p≤0.05), ** (p≤0.01), *** (p≤0.001), and **** (p≤0.0001). Furthermore, unless otherwise indicated, P1 refers to a PLP homopolymer having peptide side chains with sequence identities of SEQ ID: 1; P2 refers to a PLP homopolymer having peptide side chains with sequence identities of SEQ ID: 2; P3 refers to a PLP homopolymer having peptide side chains with sequence identities of SEQ ID: 3; P4 refers to a PLP homopolymer having peptide side chains with sequence identities of SEQ ID: 4; and HV3-TAT, HV3, HV3-TAT peptide are used interchangeably.

FIG. 1A-1B: Structure of Protein-Like Polymer (PLP). Representation of HV3 peptide analogs with different number of positively charged amino acids (FIG. 1B) polymerized into PLPs using ROMP polymerization technique (FIG. 1A).

FIG. 2A-2D: Characterization of HV3-PLPs. SEC-MALS trace results for a PLP homopolymer having peptide side chains of: FIG. 2A: SEQ ID: 1 (HVLVMSATRR), the PLP sometimes referred to herein as P1; FIG. 2B: SEQ ID: 2 (HVLVMSATKK), the PLP sometimes referred to herein as P2; FIG. 2C: SEQ ID: 3 (HVLVMSATRRRR), the PLP sometimes referred to herein as P3; and FIG. 2D: SEQ ID: 4 (HVLVMSATKKKK), the PLP sometimes referred to herein as P4.

FIG. 3A-3B: For each of FIGS. 3A-3B, P1 refers to a PLP homopolymer having peptide side chains of SEQ ID: 1 (HVLVMSATRR); P2 refers to a PLP having peptide side chains of SEQ ID: 2 (HVLVMSATKK); P3 refers to a PLP having peptide side chains of SEQ ID: 3 (HVLVMSATRRRR); P4 refers to a PLP having peptide side chains of SEQ ID: 4 (HVLVMSATKKKK); and HV3-TAT. FIG. 3A: Cell viability of HV3-TAT peptide and PLPs. Compounds treated to the HdhQ111 cells once a day for 3 days. Concentration was 3 μM with respect to peptide. FIG. 3B: VCP binding assay results for HV3-TAT, P1, P2, and P4.

FIG. 4: Live cell confocal microscopy in HdhQ111 cells showing relative cell penetration of HV3-TAT and P1 (i.e., a PLP homopolymer having peptide side chains of SEQ ID: 1). Scale bars: 60 μm; 12 μm for inset. Cell Membrane: wheat germ agglutinin-Alexa 488 (green channel). Material: Rhodamine (red channel). Nuclei: Hoechst 33342 stain (blue channel).

FIG. 5A-5B: Quantification of confocal image of FIG. 4 comparing cell penetration of HV3-TAT peptide and P1 (i.e., a PLP homopolymer having peptide side chains of SEQ ID: 1). FIG. 5B shows quantification by FACS analysis (peaks from left to right: vehicle, rhodamine, HV3-TAT-Rho, P1-Rho); and FIG. 5A shows the average of the percentage gated in FIG. 5B.

FIG. 6A-6B: In vitro blood brain barrier assay results. FIG. 6A: Schematic showing in vitro cellular assay with brain microvascular endothelial cells. FIG. 6B: % Translocation of compounds treated to HBEC-5i cells.

FIG. 7: Mitochondrial localization in live HdhQ111 cells. HdhQ111 cells were treated with HV3-TAT-Rho peptide, P1-Rho, and rhodamine dye alone at a concentration of 3 μM. Yellow (i.e., “localization” in FIG. 7) indicates colocalization of green and red channels, signifying localization to the mitochondria. Mitochondria: Mitotracker Green FM (green channel). Material: Rhodamine (red channel). Nuclei: Hoechst 33342 stain (blue channel). Scale Bars: 60 μm; 12 μm for inset.

FIG. 8: Mitochondrial fragmentation assay. The percentage of HdhQ111 cells with fragmented mitochondria relative to the total number of cells was quantitated. At least 100 cells per group were counted. P1 refers to a PLP having peptide side chains of SEQ ID: 1 and P4 refers to a PLP homopolymer having peptide side chains of SEQ ID: 4.

FIG. 9: Mitochondrial fragmentation assay. Mitochondrial morphology was determined by staining cells with anti-Tom20 antibody. Nuclei: Hoechst 33342 stain. P1 refers to a PLP having peptide side chains of SEQ ID: 1 and P4 refers to a PLP homopolymer having peptide side chains of SEQ ID: 4. Scale Bars: 60 μm; 12 μm for inset.

FIG. 10A-10B: Cell viability in HdhQ111 mouse striatal cells after enzyme pretreatment. FIG. 10A shows results for tests with HV3-TAT peptide and FIG. 10B shows results for tests with P1 (PLP having peptide side chains of SEQ ID: 1).

FIG. 11: SDS-PAGE results showing stability of PLPs after treatment with enzymes.

FIG. 12: The graph depicts percent cleavage results after treating HV3-TAT peptide and P1 with serum (10% or 25% FBS medium) and pepsin enzymes and running on HPLC.

FIG. 13: The graph depicts percent cleavage results after treating HV3-TAT peptide and P1 with liver microsomes and running on HPLC.

FIG. 14A-14B: Biolayer Interferometry (BLItz) measurements from in vitro assay evaluating binding between P1 and VCP (FIG. 14A) and between HV3-TAT peptide and VCP (FIG. 14B). FIG. 14A: lines from top to bottom: 100 μM, 10 μM, and 750 nM. FIG. 14B: lines from top to bottom: 10 μM, 7.5 μM, 5 μM, and 1.25 μM.

FIG. 15: Results from in vitro C3a assay to assess immune response (C3a complement activation) when subjected to treatment with PLPs and HV3-TAT peptide.

FIG. 16: In vitro assay results measuring percent hemolysis when subjected to treatment with PLPs and HV3-TAT peptide. Straight line at 5% hemolysis represents standards for safety.

FIG. 17: In vitro assay results measuring formation of blood clots when subject to treatment with PLPs and HV3-TAT peptide.

FIG. 18: Full pharmacokinetics of Gd-P1, where Gd-P1 refers to a PLP homopolymer having peptide side chains of SEQ ID: 1 and a peptide side chain of the contrasting agent, Gd-DOTA. n=5 per time point. The data points within the box along the vertical axis reflect the data points depicted in FIG. 19.

FIG. 19: Early range pharmacokinetics of Gd-P1, where Gd-P1 refers to a PLP homopolymer having peptide side chains of SEQ ID: 1 and a peptide side chain of the contrasting agent, Gd-DOTA. n=5 per time point. The data points in FIG. 19 correspond to the data points within the box along the vertical axis of FIG. 18.

FIG. 20: Biodistribution in the brain of Gd-P1, where Gd-P1 refers to a PLP homopolymer having peptide side chains of SEQ ID: 1 and a peptide side chain of the contrasting agent, Gd-DOTA. n=5 per time point.

FIG. 21: Optical micrographs for toxicity pathology analysis. H&E stained tissue; scale bar: 200 μm. WT C57BL/6J mice were dosed with saline or 3 mg/kg/day (with respect to peptide) of P1 (PLP homopolymer having peptide side chains of SEQ ID: 1) or HV3-TAT from 6-13 weeks of age.

FIG. 22: Quantification of in vivo results from toxicity pathology analysis as depicted in FIG. 21. Left: pathology severity scoring of H&E stained tissue; Middle: red blood cell count; Right: white blood cell count.

FIG. 23A-23D: Quantification of in vivo results from toxicity pathology analysis as depicted in FIG. 21. FIG. 23A: represents hemoglobin count; FIG. 23B: total protein count; FIG. 23C: ALT (alanine transaminase) count; FIG. 23D: total bilirubin count.

FIG. 24: Quantification of western blots of DARPP32 in striatal extracts of WT and R6/2 mice after treatment with PLP or HV3-TAT (western blot not shown). Actin was used as the loading control in the Western Blot, results are therefore depicted as ratios of DARPP32 signal relative to the actin signal in each sample.

FIG. 25: Quantification of immunohistochemistry of DARPP32 in striatum of WT and R6/2 mice after treatment with PLP or HV3-TAT (immunohistochemistry stain not shown).

FIG. 26A-26B: Polymerization kinetics and characterization of PLPs. M1 refers to a monomer comprising SEQ ID: 1; M2 refers to a monomer comprising SEQ ID: 2; M3 refers to a monomer comprising SEQ ID: 3; and M4 refers to a monomer comprising SEQ ID: 4. FIG. 26A: Percent conversion of monomers determined by integration of olefin peaks in 1H NMR. FIG. 26B: Log plots of the polymerization of each monomer. The following slopes (k_{obs}) were determined by linear lease-squares fitting of the plots: 0.0329 min⁻¹ (M1) 0.0154 min⁻¹ (M2) 0.0119 min⁻¹ (M3) 0.0076 min⁻¹ (M4).

FIG. 27: SEC-MALS trace results for batch of P1 (i.e., (PLP homopolymer having peptide side chains of SEQ ID: 1) used for all in vivo studies discussed herein.

FIG. 28A-28B: FIG. 28A: Representation of chemical structure of rhodamine labeled P1 (PLP homopolymer having peptide side chains of SEQ ID: 1). FIG. 28B: Fluorescent spectroscopy of HV3-TAT-Rho, P1-Rho, and rhodamine showing near 1:1 addition of dye on to compounds.

FIG. 29: Results from accumulation assay in HD95. Quantification of mean fluorescence output of flow cytometry in HD95 cells reporting presence of compounds 2 and 7 days post single treatment. From left to right: HV3-TAT-Rhodamine (Peptide) 3 μM; P1-Rhodamine concentration 1 μM with respect to rhodamine (27 μM with respect to peptide); P1-Rhodamine concentration 3 μM with respect to peptide (0.11 μM with respect to rhodamine); Rhodamine 3 μM; and vehicle. Unpaired t-test between groups was performed.

FIG. 30A-30E: FIG. 30A: Representation of chemical structure of EDANS-glutamic acid. FIG. 30B: Calibration curve of EDANS-glutamic acid. EDANS-glutamic acid with different concentrations was excited at 340 nm and emitted at 495 nm on a plate reader. Time course enzyme degradation of fluorogenic HV3 monomer (M1-ED) and HV3-PLP (P1-ED) by trypsin (FIG. 30C), thermolysin (FIG. 30D), and elastase (FIG. 30E). Fluorescence was measured at 495 nm by plate reader and the percent cleavage was calculated based on the calibration curve depicted in FIG. 30B.

FIG. 31A-31F: For each of FIG. 31A-31F, data is shown reflecting the results of 3 separate studies (i.e., “Batch 1+2+3”) conducted under identical conditions; “veh” refers to the vehicle saline control. For each of FIG. 31A-31C at week 6, WT: n=15; WT+P1: n=15; R62 veh: n=22; R62+HV3-TAT: n=32; and R62+P1: n=16. For each of FIG. 31D-31F at week 6, WT: n=15; WT+P1: n=15; R62 veh: n=17; R62+HV3-TAT: n=30; and R62+P1: n=13. FIG. 31A depicts the average percent of body weight gain for mice subjected to each treatment. From top to bottom, the week 13 data points correspond to: WT+P1 (˜43%); WT (˜39%); R62+P1 (˜1%); R62+HV3-TAT (˜ 4%); and R62 (˜-9%). FIG. 31B depicts the survival rate for each treatment group over time. 100% survival rate for WT veh and WT+P1. R62+P1 as compared to R62 veh: p-value=0.0454; R62+HV3-TAT as compared to R62 veh: p-value=0.0201. FIG. 31C depicts the average hindlimb clasping score for each treatment group at weeks 11, 12, and 13. For each week: R62 mice treated with vehicle (left), R62 treated with HV3-TAT (middle), and R62 treated with P1 (right). p-values in week 11: 0.0145 (top—R62 veh as compared to R62+P1), 0.0432 (middle—R62 veh as compared to R62+HV3-TAT), and 0.6051 (bottom—R62+HV3-TAT as compared to R62+P1); week 12: 0.0037 (top—R62 veh as compared to R62+P1), 0.0263 (middle—R62 veh as compared to R62+HV3-TAT), and 0.3957 (bottom—R62+HV3-TAT as compared to R62+P1); week 13: 0.0031 (top—R62 veh as compared to R62+P1), 0.0177 (middle—R62 veh as compared to R62+HV3-TAT), and 0.4938 (bottom—R62+HV3-TAT as compared to R62+P1). FIG. 31D depicts results from the open field tests representing the total distance traveled at final data collection. p-value for: WT veh as compared to R62 veh: <0.0001; WT+P1 as compared to R62 veh: <0.0001; R62 veh as compared to R62+HV3-TAT: 0.0021; R62 veh as compared to R62+P1: <0.0001; R62+HV3-TAT as compared to R62+P1: 0.1486. FIG. 31E depicts results from the horizontal activity tests. p-value for: WT veh as compared to R62 veh: <0.0001; WT+P1 as compared to R62 veh: <0.0001; R62 veh as compared to R62+HV3-TAT: 0.0262; R62 veh as compared to R62+P1: <0.0001; R62+HV3-TAT as compared to R62+P1: 0.0083. FIG. 31F depicts results from the vertical activity tests. P-value for: WT veh as compared to R62 veh: 0.0004; WT+P1 as compared to R62 veh: <0.0001; R62 veh as compared to R62+HV3-TAT: 0.2879; R62 veh as compared to R62+P1: 0.0375; R62+HV3-TAT as compared to R62+P1: 0.6222.

FIG. 32A-32C: Histograms of Quantified Western Blot Results for Protein Levels (blots not shown). Actin was used as the loading control in the Western Blot, results are therefore depicted as ratios of the protein of interest signal relative to the actin signal in each sample. FIG. 32A depicts DARPP-32 levels in striatal lysates. p-value for: WT veh as compared to R62 veh: <0.0001; WT+P1 as compared to R62 veh: <0.0001; R62 veh as compared to R62+HV3-TAT: 0.0493; R62 veh as compared to R62+P1: 0.0027; R62+P1 as compared to R62+HV3-TAT: 0.7733. FIG. 32B depicts PSD95 levels in striatal lysates. p-value for: WT veh as compared to R62 veh: 0.0034; WT+P1 as compared to R62 veh: <0.0001; R62 veh as compared to R62+HV3-TAT: 0.0191; R62 veh as compared to R62+P1: <0.0001; R62+P1 as compared to R62+HV3-TAT: 0.1442. FIG. 32C depicts BDNF levels in striatal lysates. p-value for: WT veh as compared to R62 veh: <0.0001; WT+P1 as compared to R62 veh: <0.0001; R62 veh as compared to R62+HV3-TAT: 0.0120; R62 veh as compared to R62+P1: <0.0001; R62+P1 as compared to R62+HV3-TAT: 0.0257.

FIG. 33: Quantification of immunohistochemistry of DARPP32 in striatum of WT and R6/2 mice after treatment with PLP or HV3-TAT (immunohistochemistry stain not shown). p-value for: WT veh as compared to R62 veh: 0.0010; WT+P1 as compared to R62 veh: 0.0019; R62 veh as compared to R62+HV3-TAT: 0.0496; R62 veh as compared to R62+P1: 0.0104; R62+HV3-TAT as compared to R62+P1: 0.9593.

FIG. 34A-34B: Quantification of mitochondrial studies. FIG. 34A depicts quantification of immunoblot results for VCP levels on mitochondrial fractions from striatal extracts of WT and R6/2 mice after treatment (immunoblot not shown). ATPB refers to ATP-ase β subunit. p-value for: WT veh as compared to R62 veh: <0.0001; WT+P1 as compared to R62 veh: <0.0001; R62 veh as compared to R62+HV3-TAT: 0.0001; R62 veh as compared to R62+P1: 0.0184; R62+P1 as compared to R62+HV3-TAT: 0.2663. FIG. 34B depicts quantification of mtHtt aggregation after EM48 staining (stains not shown). p-value for: R62 veh as compared to R62+HV3-TAT: 0.0491; R62 veh as compared to R62+P1: 0.0189; R62+P1 as compared to R62+HV3-TAT: 0.8765.

### STATEMENTS REGARDING CHEMICAL COMPOUNDS AND NOMENCLATURE

The following abbreviations are used herein: RP-HPLC refers to reverse-phase high performance liquid chromatography; ESI-MS refers to electrospray ionization mass spectrometry; SEC-MALS refers to size-exclusion chromatography coupled with multiangle light scattering; PLP refers to protein-like polymer; SPPS refers to solid-phase peptide synthesis; TEM refers to transmission electron microscopy; STEM refers to scanning TEM; SE or SEM refers to scanning electron microscopy; CD refers to circular dichroism; FPLC refers to fast protein liquid chromatography; FACS refers to fluorescence-activated cell sorting; MALDI-ToF MS refers to matrix assisted laser desorption ionization time of flight mass spectrometry; GPC refers to gel permeation chromatography; CLSM refers to confocal laser scanning microscopy; ICP-MS refers to inductively coupled plasma mass spectrometry; WT refers to wild-type; DP refers to degree of polymerization; VCP refers to valosin containing protein; HV3, HV-3, or HV3 peptide refers to a peptide capable of inhibiting VCP/mtHtt binding; HD refers to Huntington's Disease; Htt refers to Huntingtin protein; and mtHtt refers to mutant Huntingtin protein.

In an embodiment, a peptide, a polymer, or a composition (e.g., formulation) of the invention is isolated or purified. In an embodiment, an isolated or purified peptide, polymer, or composition (e.g., formulation) is at least partially isolated or purified as would be understood in the art. In an embodiment, the peptide, polymer, or composition (e.g., formulation) of the invention has a chemical purity of at least 95%, optionally for some applications at least 99%, optionally for some applications at least 99.9%, optionally for some applications at least 99.99%, and optionally for some applications at least 99.999% pure. The invention includes isolated and purified compositions of any of the brush block polymers described herein including the peptide brush and block copolymers and brush and brush block copolymers having one or more side chains comprising the peptide analogues, derivative, variants or fragments.

As used herein, the term “polymer” refers to a molecule composed of repeating structural units connected by covalent chemical bonds often characterized by a substantial number of repeating units (e.g., equal to or greater than 3 repeating units, optionally, in some embodiments equal to or greater than 5 repeating units, in some embodiments greater or equal to 10 repeating units) and a high molecular weight (e.g., greater than or equal to 1 kDa, in some embodiments greater than or equal to 5 kDa or greater than or equal to 50 kDa). Polymers are commonly the polymerization product of one or more monomer precursors. The term polymer includes homopolymers, or polymers consisting essentially of a single repeating monomer subunit. The term polymer also includes copolymers which are formed when two or more different types of monomers are linked in the same polymer. Copolymers may comprise two or more monomer subunits (e.g., 3 or more monomer subunits, 4 or more monomer subunits, 5 or more monomer subunits, or 6 or more monomer subunits), and include random, block, brush, brush block, alternating, segmented, grafted, tapered and other architectures. In some embodiments, copolymers of the invention comprise from 2 to 10 different monomer subunits. Useful polymers include organic polymers that may be in amorphous, semi-amorphous, crystalline or semi-crystalline states. Cross linked polymers having linked monomer chains are useful for some applications, for example linked by one or more disulfide linkages. The invention provides polymers comprising therapeutic agents, such as brush polymers having at least a portion of the repeating units comprising polymer side chains such as peptide side chains.

An “oligomer” refers to a molecule composed of repeating structural units connected by covalent chemical bonds often characterized by a number of repeating units less than that of a polymer (e.g., equal to or less than 3 repeating units) and a lower molecular weights (e.g., less than or equal to 1,000 Da) than polymers. Oligomers may be the polymerization product of one or more monomer precursors.

A “peptide” or “oligopeptide” herein are used interchangeably and refer to a polymer of repeating structural units connected by a peptide bond. Typically, the repeating structural units of the peptide are amino acids including naturally occurring amino acids, non-naturally occurring amino acids, analogues of amino acids or any combination of these. The number of repeating structural units of a peptide, as understood in the art, are typically less than a “protein”, and thus the peptide often has a lower molecular weight than a protein. In some embodiments, a peptide has a chain length of 3 to 150 amino acids, optionally 3 to 100 amino acids, optionally 5 to 50 amino acids, and optionally 5 to 30 amino acids.

“Block copolymers” are a type of copolymer comprising blocks or spatially segregated domains, wherein different domains comprise different polymerized monomers, for example, including at least two chemically distinguishable blocks. Block copolymers may further comprise one or more other structural domains, such as hydrophobic groups, hydrophilic groups, etc. In a block copolymer, adjacent blocks are constitutionally different, i.e., adjacent blocks comprise constitutional units derived from different species of monomer or from the same species of monomer but with a different composition or sequence distribution of constitutional units. Different blocks (or domains) of a block copolymer may reside on different ends or the interior of a polymer (e.g., [A][B]), or may be provided in a selected sequence ([A][B][A][B]). “Diblock copolymer” refers to block copolymer having two different polymer blocks. “Triblock copolymer” refers to a block copolymer having three different polymer blocks, including compositions in which two non-adjacent blocks are the same or similar. “Pentablock” copolymer refers to a copolymer having five different polymer including compositions in which two or more non-adjacent blocks are the same or similar.

“Statistical copolymers,” also generally known in the art as “random copolymers,” are copolymers in which the ordering of backbone groups is dictated by reaction kinetics. Statistical copolymers generally are antithetical to block copolymers.

“Polymer backbone group” or “polymer backbone subunit” refers to groups that are covalently linked to make up a backbone of a polymer, such as a block copolymer. Polymer backbone groups may be linked to side chain groups, such as polymer side chain groups. Some polymer backbone groups useful in the present compositions are derived from polymerization of a monomer selected from the group consisting of a substituted or unsubstituted norbornene, olefin, cyclic olefin, norbornene anhydride, cyclooctene, cyclopentadiene, styrene, acrylamide, and acrylate. Some polymer backbone groups useful in the present compositions are obtained from a ring opening metathesis polymerization (ROMP) reaction. Polymer backbones may terminate in a range of backbone terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen, C₁-C₁₀ alkyl or C₅-C₁₀ aryl.

“Polymer side chain group” (also sometimes referred to herein as “substituent,” e.g., with respect to R¹) refers to a group covalently linked (directly or indirectly) to a polymer backbone group that comprises a polymer side chain, optionally imparting steric properties to the polymer. In an embodiment, for example, a polymer side chain group is characterized by a plurality of repeating units having the same, or similar, chemical composition. A polymer side chain group may be directly or indirectly linked to the polymer back bone groups. In some embodiments, polymer side chain groups provide steric bulk and/or interactions that result in an extended polymer backbone and/or a rigid polymer backbone. Some polymer side chain groups useful in the present compositions include unsubstituted or substituted peptide groups. Some polymer side chain groups useful in the present compositions comprise repeating units obtained via anionic polymerization, cationic polymerization, free radical polymerization, group transfer polymerization, or ring-opening polymerization. A polymer side chain may terminate in a wide range of polymer side chain terminating groups including hydrogen, C₁-C₁₀ alkyl, C₃-C₁₀ cycloalkyl, C₅-C₁₀ aryl, C₅-C₁₀ heteroaryl, C₁-C₁₀ acyl, C₁-C₁₀ hydroxyl, C₁-C₁₀ alkoxy, C₂-C₁₀ alkenyl, C₂-C₁₀ alkynyl, C₅-C₁₀ alkylaryl, —CO₂R³⁰, —CONR³¹R³², —COR³³, —SOR³⁴, —OSR³⁵, —SO₂R³⁶, —OR³⁷, —SR³⁸, —NR³⁹R⁴⁰, —NR⁴¹COR⁴², C₁-C₁₀ alkyl halide, phosphonate, phosphonic acid, silane, siloxane, acrylamide, acrylate, or catechol; wherein each of R³⁰-R⁴² is independently hydrogen or C₁-C₅ alkyl.

As used herein, the term “polymer segment” (e.g., first polymer segment, second polymer segment, etc.) refers to a section (e.g., portion) of the polymer comprising a particular monomer or arrangement of monomers. A polymer segment can be a homopolymer or a copolymer. In embodiments where a polymer segment is a copolymer, the copolymer can exist in any suitable arrangement of monomers (e.g., random, block, brush, brush block, alternating, segmented, grafted, tapered, statistical and other architectures). In some embodiments, the polymer segments are homopolymers, random copolymers, statistical copolymers, or block copolymers. Any polymer (e.g., brush polymer) described herein can have a single polymer segment or multiple polymer segments. In embodiments where the polymer has multiple polymer segments, the polymer segments can exist in any suitable arrangement (random, block, brush, brush block, alternating, segmented, grafted, tapered, statistical, and other architectures).

As used herein, the term “degree of polymerization” refers to the average number of monomer units per polymer chain. For example, for certain polymers described herein, comprising B¹, B², and/or B³ backbone units, the degree of polymerization would be represented by the sum total of B¹, B², and B³ backbone units. Since the degree of polymerization can vary from polymer to polymer, the degree of polymerization is generally represented by an average.

As used herein, the term “brush polymer” refers to a polymer comprising repeating units each independently comprising a polymer backbone group covalently linked to at least one polymer side chain group. A brush polymer may be characterized by brush density which refers to the percentage of the repeating units comprising polymer side chain groups. Brush polymers of certain aspects are characterized by a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects are characterized by a brush density selected from the range 50% to 100%, optionally some embodiments a density selected from the range of 75% to 100%, or optionally for some embodiments a density selected from the range of 90% to 100%. Brush polymers, such as the polymers disclosed herein (e.g., a polymer of formula (1)), can be prepared by any suitable methods including, “grafting from” methods, “grafting onto” methods, “grafting through” methods, or any combination thereof. Such suitable methods can include, for example, ring opening metathesis polymerization (ROMP) synthetic pathways and/or non-ROMP synthetic pathways, such as, by way of example, reversible addition fragmentation chain transfer (RAFT) polymerization, stable free radical mediated polymerization and atom transfer radical polymerization (ATRP).

As used herein, the term “peptide density” refers to the percentage of monomer units in the polymer chain which have a peptide covalently linked thereto, and such “peptide density” can be calculated generally for all peptides or for a specific peptide. The percentage is based on the overall sum of monomer units in the polymer chain. For example, for certain polymers described herein, the density of peptide P¹ (or percentage of monomer units comprising peptide P¹) in a polymer having m repeat units of peptide P¹, o repeat units of B²-R¹, and n repeat units of peptide P², is represented by the formula:

$$\frac{m}{m + n + o} \times 100 ,$$

where each variable refers to the number of monomer units of that type in the polymer chain. Polymers of certain aspects are characterized by a peptide density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Polymers of certain aspects are characterized by a peptide density selected from the range 50% to 100%, optionally some embodiments a density selected from the range of 75% to 100%, or optionally for some embodiments a density selected from the range of 90% to 100%. In some embodiments, the brush density is equal to the peptide density.

In an aspect, the polymer side chain groups (e.g., also termed substituents herein) can have any suitable spacing on the polymer backbone. Typically, the space between adjacent polymer side chain groups is from 3 angstroms to 30 angstroms, and optionally 5 to 20 angstroms and optionally 5 to 10 angstroms. By way of illustration, in certain embodiments having a brush density of 100%, the polymer side chain groups typically are spaced 6±5 angstroms apart on the polymer backbone. In some embodiments the brush polymer has a high a brush density (e.g., greater than 70%), wherein the polymer side chain groups are spaced 5 to 20 angstroms apart on the polymer backbone.

As used herein, the term “sequence homology” or “sequence identity” means the proportion of amino acid matches between two amino acid sequences of interest in two different peptides considering the ordering of the amino acids. Matches occur when amino acids are in the same order in one peptide compared to the other peptide. When sequence homology is expressed as a percentage, e.g., 50%, the percentage denotes the fraction of matches over the length of sequence that is compared to some other sequence, considering the amino acid order. Gaps (in either of the two sequences) are permitted to maximize matching; for example, wherein gap lengths of 5 amino acids or less, optionally 3 amino acids or less, are usually used. In other words, a sequence having 75% or greater sequence identity to an amino acid sequence with 9 amino acids can indicate that the 9 amino acid sequence can have one or two point mutations (i.e., amino acid change), one or two amino acid deletions, one or two amino acid additions, one point mutation and one amino acid deletion, or one point mutation and one amino acid addition. Even with two such amino acids being different, 7 out of 9 amino acids still match in the correct order, such that there is greater than 75% sequence identity. For clarity, the analysis of whether there sequence homology between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

As used herein, the term “amino acid composition similarity” or “amino acid similarity” means the proportion of amino acid matches between two amino acid sequences of interest in two different peptides regardless of the ordering of the amino acids. Matches occur when amino acids are present in both amino acid sequences regardless of order. When amino acid composition similarity is expressed as a percentage, e.g., 50%, the percentage denotes the fraction of matches over the length of sequence that is compared to some other sequence, regardless of amino acid order. Gaps (in either of the two sequences) are permitted to maximize matching; for example, wherein gap lengths of 5 amino acids or less, optionally 3 amino acids or less, are usually used. By way of example, if two amino acid sequences each containing ten amino acids have three amino acids in common, in any order, then there is 30% amino acid composition similarity between the sequences. For clarity, the analysis of whether there is amino acid composition similarity between two amino acid sequences of interest is conducted with respect to a particular portion of one peptide or protein (i.e., a first amino acid sequence of interest) relative to a particular portion of another peptide or protein (i.e., a second amino acid sequence of interest), and is not conducted relative to all amino acids present in a peptide or protein (i.e., the analysis does not include amino acids outside of the particular amino acid sequence of interest).

The term “fragment” refers to a portion, but not all of, a composition or material, such as a peptide composition or material. In an embodiment, a fragment of a peptide refers to 50% or more of the sequence of amino acids, optionally 70% or more of the sequence of amino acids and optionally 90% or more of the sequence of amino acids.

“Polymer blend” refers to a mixture comprising at least one polymer, such as a brush polymer, e.g., brush block copolymer, and at least one additional component, and optionally more than one additional component. In some embodiments, for example, a polymer blend of the invention comprises a first brush copolymer and one or more addition brush polymers having a composition different than the first brush copolymer. In some embodiments, for example, a polymer blend of the invention further comprises one or more additional brush block copolymers, homopolymers, copolymers, block copolymers, brush block copolymers, oligomers, solvent, small molecules (e.g., molecular weight less than 500 Da, optionally less than 100 Da), or any combination of these. Polymer blends useful for some applications comprise a first brush polymer, and one or more additional components comprising polymers, block copolymers, brush polymers, linear block copolymers, random copolymers, homopolymers, or any combinations of these. Polymer blends of the invention include mixture of two, three, four, five and more polymer components.

As used herein, the term “compound” can be used to refer to any of the peptides or polymers described herein. Alternatively, or additionally, the term compound can refer to any of the synthetic precursors, reagents, additives, excipients, etc. used in preparation of or formulation with the peptides or polymers described herein.

As used herein, the term “group” may refer to a functional group of a chemical compound. Groups of the present compounds refer to an atom or a collection of atoms that are a part of the compound. Groups of the present invention may be attached to other atoms of the compound via one or more covalent bonds. Groups may also be characterized with respect to their valence state. The present invention includes groups characterized as monovalent, divalent, trivalent, etc. valence states.

As used herein, the term “substituted” generally refers to a compound wherein a hydrogen is replaced by another functional group, unless otherwise contradicted by context.

Unless otherwise specified, the term “average molecular weight” or “molecular weight” refers to number average molecular weight. Number average molecular weight is the defined as the total weight of a sample volume divided by the number of molecules within the sample. As is customary and well known in the art, peak average molecular weight and weight average molecular weight may also be used to characterize the molecular weight of the distribution of polymers within a sample.

As used herein, “mimic,” “mimicking,” “mimetic,” and grammatically equivalent variations in reference to a compound, oligomer, and/or polymer mimicking a given species (e.g., “proteomimetic”), such as one or more oligo- or poly-peptides (e.g., proteins), means that the compound, oligomer, and/or polymer has a portion with a similar and/or corresponding amino acid sequence to a portion of the given species. In some aspects, the similar and/or corresponding portion typically relates to there being a certain level of sequence homology and/or amino acid composition similarity between the given species and the one or more oligo- or poly-peptides (e.g., proteins). In aspects, a mimetic refers to a material capable of imitating key structures and/or functions of a peptide or protein. Mimetics may be synthetically produced and modified to comprise specific properties depending on desired outcome, including variable size, greater stability, greater affinity, protease-resistance and improved solubility. In aspects, mimetic refers to a protein-like polymer (PLP) designed to engage proteins and the quality control machinery within cells. Callmann C E et al., Poly(peptide): Synthesis, Structure, and Function of Peptide-Polymer Amphiphiles and Protein-like Polymers. Acc Chem Res 2020; 53:400-13; Gianneschi N C et al., Biomolecular Densely Grafted Brush Polymers: Oligonucleotides, Oligosaccharides and Oligopeptides. Angew Chemie Int Ed 2020; Blum A P, Kammeyer J K, Gianneschi N C, each of which is incorporated by reference herein in its entirety, and more specifically to facilitate the understanding of PLPs, to the extent not inconsistent with the description herein.

In aspects of the invention, a mimetic may be modified to comprise a residue-specific modification, a peptide backbone modification, an N-terminal modification, a C-terminal modification, or any combination thereof. In examples, the modification may improve peptide stability, alter peptide structure, incorporate imaging and/or detection agents, improve solubility, enhance non-specific enzyme resistance, reduce steric hindrance, increase cellular penetration, improve binding affinities to targets, enhance safety, or any combination thereof. For example, a modification may include one or more of: biotin labeling, contrast agent labeling such as Gd-DOTA labeling, fluorescent dye labeling such as cyanine labeling, fluorescein and 7-methoxycoumarin acetic acid labeling, dansyl and/or 2,4-dinitrophenyl labeling, EDANS labeling, coumarin labeling, and/or rhodamine labeling, one or more point mutations, introduction of one or more spacers, isotopic labeling, introduction of one or more chelating agents, acetylation, amidation, methylation, palmitylation, hydroxylation, glycosylation, sulfation and sulfonation, esterification, phosphorylation, peptide stapling, lipidation, cyclization, or any combination thereof.

As used herein, the phrase “charge modulating domain” refers to one or more amino acids added to the peptide sequences described herein to modulate the charge of the peptide. For example, the charge modulating domain can be a TAT sequence, a glycine-serine domain, a cationic residue domain, or a combination thereof, or optionally a glycine-serine domain, a cationic residue domain, or a combination thereof. In certain embodiments, the charge modulating domain has from 2 to 7 amino acid residues. The 2 to 7 amino acids can be added in a single block containing from 2 to 7 amino acid residues or more than one block containing from 1 to 6 amino acid residues. In some embodiments, the charge modulating domain is a cationic residue domain having from 2 to 7 amino acid residues selected from lysine, arginine, histidine, or a combination thereof. In some embodiments, the charge modulating domain comprises an aspartic acid residue. Generally, the charge modulating domain modulates the charge of the peptide to have a net positive charge. Without wishing to be bound by any particular theory, it is believed that the net positive charge increases the cellular uptake of the peptide or polymer comprising the peptide. The overall charge of the peptide or copolymer comprising the peptide can be determined by any suitable means. For example, the overall charge can be determined by (i) structural analysis of the functional residues on the peptide sequence and their respective pKa, (ii) physical characterization by measuring the zeta potential, and/or (iii) by virtue of the material moving towards a negative pole in an electrophoresis polymer gel. In certain embodiments, the overall charge of the peptide or copolymer comprising the peptide is determined by measuring the zeta potential.

As used herein, the terms “alkylene” and “alkylene group” are used synonymously and refer to a divalent group derived from an alkyl group as defined herein. The invention includes compounds having one or more alkylene groups. Alkylene groups in some compounds function as linking and/or spacer groups. Compounds of the invention may have substituted and/or unsubstituted C₁-C₂₀ alkylene, C₁-C₁₀ alkylene and C₁-C₅ alkylene groups, for example, as one or more linking groups (e.g., L¹, L²).

As used herein, the terms “cycloalkylene” and “cycloalkylene group” are used synonymously and refer to a divalent group derived from a cycloalkyl group as defined herein. The invention includes compounds having one or more cycloalkylene groups. Cycloalkyl groups in some compounds function as linking and/or spacer groups. Compounds of the invention may have substituted and/or unsubstituted C₃-C₂₀ cycloalkylene, C₃-C₁₀ cycloalkylene and C₃-C₅ cycloalkylene groups, for example, as one or more linking groups (e.g., L¹, L²).

As used herein, the terms “arylene” and “arylene group” are used synonymously and refer to a divalent group derived from an aryl group as defined herein. The invention includes compounds having one or more arylene groups. In some embodiments, an arylene is a divalent group derived from an aryl group by removal of hydrogen atoms from two intra-ring carbon atoms of an aromatic ring of the aryl group. Arylene groups in some compounds function as linking and/or spacer groups. Arylene groups in some compounds function as chromophore, fluorophore, aromatic antenna, dye and/or imaging groups. Compounds of the invention include substituted and/or unsubstituted C₃-C₃₀ arylene, C₃-C₂₀ arylene, C₃-C₁₀ arylene and C₁-C₅ arylene groups, for example, as one or more linking groups (e.g., L¹, L²).

As used herein, the terms “heteroarylene” and “heteroarylene group” are used synonymously and refer to a divalent group derived from a heteroaryl group as defined herein. The invention includes compounds having one or more heteroarylene groups. In some embodiments, a heteroarylene is a divalent group derived from a heteroaryl group by removal of hydrogen atoms from two intra-ring carbon atoms or intra-ring nitrogen atoms of a heteroaromatic or aromatic ring of the heteroaryl group. Heteroarylene groups in some compounds function as linking and/or spacer groups. Heteroarylene groups in some compounds function as chromophore, aromatic antenna, fluorophore, dye and/or imaging groups. Compounds of the invention include substituted and/or unsubstituted C₃-C₃₀ heteroarylene, C₃-C₂₀ heteroarylene, C₁-C₁₀ heteroarylene and C₃-C₅ heteroarylene groups, for example, as one or more linking groups (e.g., L¹, L²).

As used herein, the terms “alkenylene” and “alkenylene group” are used synonymously and refer to a divalent group derived from an alkenyl group as defined herein. The invention includes compounds having one or more alkenylene groups. Alkenylene groups in some compounds function as linking and/or spacer groups. Compounds of the invention include substituted and/or unsubstituted C₂-C₂₀ alkenylene, C₂-C₁₀ alkenylene and C₂-C₅ alkenylene groups, for example, as one or more linking groups (e.g., L¹, L²).

As used herein, the terms “cycloalkenylene” and “cycloalkenylene group” are used synonymously and refer to a divalent group derived from a cycloalkenyl group as defined herein. The invention includes compounds having one or more cycloalkenylene groups. Cycloalkenylene groups in some compounds function as linking and/or spacer groups. Compounds of the invention include substituted and/or unsubstituted C₃-C₂₀ cycloalkenylene, C₃-C₁₀ cycloalkenylene and C₃-C₅ cycloalkenylene groups, for example, as one or more linking groups (e.g., L¹, L²).

As used herein, the terms “alkynylene” and “alkynylene group” are used synonymously and refer to a divalent group derived from an alkynyl group as defined herein. The invention includes compounds having one or more alkynylene groups. Alkynylene groups in some compounds function as linking and/or spacer groups. Compounds of the invention include substituted and/or unsubstituted C₂-C₂₀ alkynylene, C₂-C₁₀ alkynylene and C₂-C₅ alkynylene groups, for example, as one or more linking groups (e.g., L¹, L²).

As used herein, the term “halo” refers to a halogen group such as a fluoro (—F), chloro (—Cl), bromo (—Br), iodo (—I) or astato (—At).

The term “heterocyclic” refers to ring structures containing at least one other kind of atom, in addition to carbon, in the ring. Examples of such heteroatoms include nitrogen, oxygen and sulfur. Heterocyclic rings include heterocyclic alicyclic rings and heterocyclic aromatic rings. Examples of heterocyclic rings include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups. Atoms of heterocyclic rings can be bonded to a wide range of other atoms and functional groups, for example, provided as substituents.

The term “carbocyclic” refers to ring structures containing only carbon atoms in the ring. Carbon atoms of carbocyclic rings can be bonded to a wide range of other atoms and functional groups, for example, provided as substituents.

The term “alicyclic ring” refers to a ring, or plurality of fused rings, that is not an aromatic ring. Alicyclic rings include both carbocyclic and heterocyclic rings.

The term “aromatic ring” refers to a ring, or a plurality of fused rings, that includes at least one aromatic ring group. The term aromatic ring includes aromatic rings comprising carbon, hydrogen and heteroatoms. Aromatic ring includes carbocyclic and heterocyclic aromatic rings. Aromatic rings are components of aryl groups.

The term “fused ring” or “fused ring structure” refers to a plurality of alicyclic and/or aromatic rings provided in a fused ring configuration, such as fused rings that share at least two intra ring carbon atoms and/or heteroatoms.

As used herein, the term “alkoxyalkyl” refers to a substituent of the formula alkyl-O-alkyl.

As used herein, the term “polyhydroxyalkyl” refers to a substituent having from 2 to 12 carbon atoms and from 2 to 5 hydroxyl groups, such as the 2,3-dihydroxypropyl, 2,3,4-trihydroxybutyl or 2,3,4,5-tetrahydroxypentyl residue.

As used herein, the term “polyalkoxyalkyl” refers to a substituent of the formula alkyl-(alkoxy)ₙ-alkoxy wherein n is an integer from 1 to 10, preferably 1 to 4, and more preferably for some embodiments 1 to 3.

Amino acids include glycine, alanine, valine, leucine, isoleucine, methionine, proline, phenylalanine, tryptophan, asparagine, glutamine, glycine, serine, threonine, serine, threonine, glutamine, tyrosine, cysteine, lysine, arginine, histidine, aspartic acid and glutamic acid. As used herein, reference to “a side chain residue of a natural α-amino acid” specifically includes the side chains of the above-referenced amino acids. Peptides are comprised of two or more amino acids connected via peptide bonds.

Alkyl groups include straight-chain, branched and cyclic alkyl groups. Alkyl groups include those having from 1 to 30 carbon atoms. Alkyl groups include small alkyl groups having 1 to 3 carbon atoms. Alkyl groups include medium length alkyl groups having from 4-10 carbon atoms. Alkyl groups include long alkyl groups having more than 10 carbon atoms, particularly those having 10-30 carbon atoms. The term cycloalkyl specifically refers to an alky group having a ring structure such as ring structure comprising 3-30 carbon atoms, optionally 3-20 carbon atoms and optionally 2-10 carbon atoms, including an alkyl group having one or more rings. Cycloalkyl groups include those having a 3-, 4-, 5-, 6-, 7-, 8-, 9- or 10-member carbon ring(s) and particularly those having a 3-, 4-, 5-, 6-, or 7-member ring(s). The carbon rings in cycloalkyl groups can also carry alkyl groups. Cycloalkyl groups can include bicyclic and tricycloalkyl groups. Alkyl groups are optionally substituted. Substituted alkyl groups include among others those which are substituted with aryl groups, which in turn can be optionally substituted. Specific alkyl groups include methyl, ethyl, n-propyl, iso-propyl, cyclopropyl, n-butyl, s-butyl, t-butyl, cyclobutyl, n-pentyl, branched-pentyl, cyclopentyl, n-hexyl, branched hexyl, and cyclohexyl groups, all of which are optionally substituted. Substituted alkyl groups include fully halogenated or semihalogenated alkyl groups, such as alkyl groups having one or more hydrogens replaced with one or more fluorine atoms, chlorine atoms, bromine atoms and/or iodine atoms. Substituted alkyl groups include fully fluorinated or semifluorinated alkyl groups, such as alkyl groups having one or more hydrogens replaced with one or more fluorine atoms. An alkoxy group is an alkyl group that has been modified by linkage to oxygen and can be represented by the formula R—O and can also be referred to as an alkyl ether group. Examples of alkoxy groups include, but are not limited to, methoxy, ethoxy, propoxy, butoxy and heptoxy. Alkoxy groups include substituted alkoxy groups wherein the alky portion of the groups is substituted as provided herein in connection with the description of alkyl groups. As used herein MeO— refers to CH₃O—. Compositions of some embodiments of the invention comprise alkyl groups as terminating groups, such as polymer backbone terminating groups and/or polymer side chain terminating groups.

Alkenyl groups include straight-chain, branched and cyclic alkenyl groups. Alkenyl groups include those having 1, 2 or more double bonds and those in which two or more of the double bonds are conjugated double bonds. Alkenyl groups include those having from 2 to 20 carbon atoms. Alkenyl groups include small alkenyl groups having 2 to 3 carbon atoms. Alkenyl groups include medium length alkenyl groups having from 4-10 carbon atoms. Alkenyl groups include long alkenyl groups having more than 10 carbon atoms, particularly those having 10-20 carbon atoms. Cycloalkenyl groups include those in which a double bond is in the ring or in an alkenyl group attached to a ring. The term cycloalkenyl specifically refers to an alkenyl group having a ring structure, including an alkenyl group having a 3-, 4-, 5-, 6-, 7-, 8-, 9- or 10-member carbon ring(s) and particularly those having a 3-, 4-, 5-, 6- or 7-member ring(s). The carbon rings in cycloalkenyl groups can also carry alkyl groups. Cycloalkenyl groups can include bicyclic and tricyclic alkenyl groups. Alkenyl groups are optionally substituted. Substituted alkenyl groups include among others those which are substituted with alkyl or aryl groups, which groups in turn can be optionally substituted. Specific alkenyl groups include ethenyl, prop-1-enyl, prop-2-enyl, cycloprop-1-enyl, but-1-enyl, but-2-enyl, cyclobut-1-enyl, cyclobut-2-enyl, pent-1-enyl, pent-2-enyl, branched pentenyl, cyclopent-1-enyl, hex-1-enyl, branched hexenyl, cyclohexenyl, all of which are optionally substituted. Substituted alkenyl groups include fully halogenated or semihalogenated alkenyl groups, such as alkenyl groups having one or more hydrogens replaced with one or more fluorine atoms, chlorine atoms, bromine atoms and/or iodine atoms. Substituted alkenyl groups include fully fluorinated or semifluorinated alkenyl groups, such as alkenyl groups having one or more hydrogen atoms replaced with one or more fluorine atoms. Compositions of some embodiments of the invention comprise alkenyl groups as terminating groups, such as polymer backbone terminating groups and/or polymer side chain terminating groups.

Aryl groups include groups having one or more 5-, 6- or 7-member aromatic rings, including heterocyclic aromatic rings. The term heteroaryl specifically refers to aryl groups having at least one 5-, 6- or 7-member heterocyclic aromatic rings. Aryl groups can contain one or more fused aromatic rings, including one or more fused heteroaromatic rings, and/or a combination of one or more aromatic rings and one or more nonaromatic rings that may be fused or linked via covalent bonds. Heterocyclic aromatic rings can include one or more N, O, or S atoms in the ring. Heterocyclic aromatic rings can include those with one, two or three N atoms, those with one or two O atoms, and those with one or two S atoms, or combinations of one or two or three N, O or S atoms. Aryl groups are optionally substituted. Substituted aryl groups include among others those which are substituted with alkyl or alkenyl groups, which groups in turn can be optionally substituted. Specific aryl groups include phenyl, biphenyl groups, pyrrolidinyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, and naphthyl groups, all of which are optionally substituted. Substituted aryl groups include fully halogenated or semihalogenated aryl groups, such as aryl groups having one or more hydrogens replaced with one or more fluorine atoms, chlorine atoms, bromine atoms and/or iodine atoms. Substituted aryl groups include fully fluorinated or semifluorinated aryl groups, such as aryl groups having one or more hydrogens replaced with one or more fluorine atoms. Aryl groups include, but are not limited to, aromatic group-containing or heterocylic aromatic group-containing groups corresponding to any one of the following: benzene, naphthalene, naphthoquinone, diphenylmethane, fluorene, anthracene, anthraquinone, phenanthrene, tetracene, tetracenedione, pyridine, quinoline, isoquinoline, indoles, isoindole, pyrrole, imidazole, oxazole, thiazole, pyrazole, pyrazine, pyrimidine, purine, benzimidazole, furans, benzofuran, dibenzofuran, carbazole, acridine, acridone, phenanthridine, thiophene, benzothiophene, dibenzothiophene, xanthene, xanthone, flavone, coumarin, azulene or anthracycline. As used herein, a group corresponding to the groups listed above expressly includes an aromatic or heterocyclic aromatic group, including monovalent, divalent and polyvalent groups, of the aromatic and heterocyclic aromatic groups listed herein are provided in a covalently bonded configuration in the compounds of the invention at any suitable point of attachment. In embodiments, aryl groups contain between 5 and 30 carbon atoms. In embodiments, aryl groups contain one aromatic or heteroaromatic six-membered ring and one or more additional five- or six-membered aromatic or heteroaromatic ring. In embodiments, aryl groups contain between five and eighteen carbon atoms in the rings. Aryl groups optionally have one or more aromatic rings or heterocyclic aromatic rings having one or more electron donating groups, electron withdrawing groups and/or targeting ligands provided as substituents. Compositions of some embodiments of the invention comprise aryl groups as terminating groups, such as polymer backbone terminating groups and/or polymer side chain terminating groups.

Arylalkyl groups are alkyl groups substituted with one or more aryl groups wherein the alkyl groups optionally carry additional substituents and the aryl groups are optionally substituted. Specific alkylaryl groups are phenyl-substituted alkyl groups, e.g., phenylmethyl groups. Alkylaryl groups are alternatively described as aryl groups substituted with one or more alkyl groups wherein the alkyl groups optionally carry additional substituents and the aryl groups are optionally substituted. Specific alkylaryl groups are alkyl-substituted phenyl groups such as methylphenyl. Substituted arylalkyl groups include fully halogenated or semihalogenated arylalkyl groups, such as arylalkyl groups having one or more alkyl and/or aryl groups having one or more hydrogens replaced with one or more fluorine atoms, chlorine atoms, bromine atoms and/or iodine atoms. Compositions of some embodiments of the invention comprise arylalkyl groups as terminating groups, such as polymer backbone terminating groups and/or polymer side chain terminating groups.

As to any of the groups described herein which contain one or more substituents, it is understood that such groups do not contain any substitution or substitution patterns which are sterically impractical and/or synthetically non-feasible. Optional substitution of alkyl groups includes substitution with one or more alkenyl groups, aryl groups or both, wherein the alkenyl groups or aryl groups are optionally substituted. Optional substitution of alkenyl groups includes substitution with one or more alkyl groups, aryl groups, or both, wherein the alkyl groups or aryl groups are optionally substituted. Optional substitution of aryl groups includes substitution of the aryl ring with one or more alkyl groups, alkenyl groups, or both, wherein the alkyl groups or alkenyl groups are optionally substituted.

Optional substituents for any alkyl, alkenyl and aryl group includes substitution with one or more of the following substituents, among others: halogen, including fluorine, chlorine, bromine or iodine; pseudohalides, including —CN;

—COOR where R is a hydrogen or an alkyl group or an aryl group and more specifically where R is a methyl, ethyl, propyl, butyl, or phenyl group all of which groups are optionally substituted;

—COR where R is a hydrogen or an alkyl group or an aryl group and more specifically where R is a methyl, ethyl, propyl, butyl, or phenyl group all of which groups are optionally substituted;

—CON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—OCON(R)₂ where each R, independently of each other R, is a hydrogen or an alkyl group or an aryl group and more specifically where R is a methyl, ethyl, propyl, butyl, or phenyl group all of which groups are optionally substituted; and where R and R can form a ring which can contain one or more double bonds and can contain one or more additional carbon atoms;

—N(R)₂ where each R, independently of each other R, is a hydrogen, or an alkyl group, or an acyl group or an aryl group and more specifically where R is a methyl, ethyl, propyl, butyl, phenyl or acetyl group, all of which are optionally substituted; and where R and R can form a ring which can contain one or more double bonds and can contain one or more additional carbon atoms;

—SR, where R is hydrogen or an alkyl group or an aryl group and more specifically where R is hydrogen, methyl, ethyl, propyl, butyl, or a phenyl group, which are optionally substituted;

—SO₂R, or —SOR where R is an alkyl group or an aryl group and more specifically where R is a methyl, ethyl, propyl, butyl, or phenyl group, all of which are optionally substituted;

—OCOOR where R is an alkyl group or an aryl group;

—SO₂N(R)₂ where each R, independently of each other R, is a hydrogen, or an alkyl group, or an aryl group all of which are optionally substituted and wherein R and R can form a ring which can contain one or more double bonds and can contain one or more additional carbon atoms;

—OR where R is H, an alkyl group, an aryl group, or an acyl group all of which are optionally substituted. In a particular example R can be an acyl yielding —OCOR″ where R″ is a hydrogen or an alkyl group or an aryl group and more specifically where R″ is methyl, ethyl, propyl, butyl, or phenyl groups all of which groups are optionally substituted.

Specific substituted alkyl groups include haloalkyl groups, particularly trihalomethyl groups and specifically trifluoromethyl groups. Specific substituted aryl groups include mono-, di-, tri, tetra- and pentahalo-substituted phenyl groups; mono-, di-, tri-, tetra-, penta-, hexa-, and hepta-halo-substituted naphthalene groups; 3- or 4-halo-substituted phenyl groups, 3- or 4-alkyl-substituted phenyl groups, 3- or 4-alkoxy-substituted phenyl groups, 3- or 4-RCO-substituted phenyl, 5- or 6-halo-substituted naphthalene groups. More specifically, substituted aryl groups include acetylphenyl groups, particularly 4-acetylphenyl groups; fluorophenyl groups, particularly 3-fluorophenyl and 4-fluorophenyl groups; chlorophenyl groups, particularly 3-chlorophenyl and 4-chlorophenyl groups; methylphenyl groups, particularly 4-methylphenyl groups; and methoxyphenyl groups, particularly 4-methoxyphenyl groups.

As to any of the above groups which contain one or more substituents, it is understood that such groups do not contain any substitution or substitution patterns which are sterically impractical and/or synthetically non-feasible.

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, which is combined with buffer prior to use.

Thus, the compounds, oligomers, or polymers disclosed herein may exist as salts, such as with pharmaceutically acceptable acids. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds, oligomers, or polymers disclosed herein can exist in unsolvated forms as well as solvated forms, including hydrated forms. In general, the solvated forms are equivalent to unsolvated forms and are encompassed within the scope of the present invention. Certain compounds disclosed herein may exist in multiple crystalline or amorphous forms. In general, all physical forms are equivalent for the uses contemplated herein and are intended to be within the scope of the disclosed compounds, oligomers, or polymers.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Certain compounds of the present invention possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as D- or L- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or D- or L-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms. Isomers include structural isomers and stereoisomers such as enantiomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

As is customary and well known in the art, hydrogen atoms in formulas (FX1), (FX2), and (RU1)-(RU3c) are not always explicitly shown, for example, hydrogen atoms bonded to the carbon atoms of aromatic, heteroaromatic, and alicyclic rings are not always explicitly shown in formulas (FX1), (FX2), and (RU1)-(RU3c). The structures provided herein, for example in the context of the description of formulas (FX1), (FX2), and (RU1)-(RU3c) and schematics and structures in the drawings, are intended to convey to one of reasonable skill in the art the chemical composition of compounds of the methods and compositions of the invention, and as will be understood by one of skill in the art, the structures provided do not indicate the specific positions and/or orientations of atoms and the corresponding bond angles between atoms of these compounds.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “treating” or “treatment” refers to any indicia of success in the treatment or amelioration of an injury, disease, pathology or condition, including any objective or subjective parameter such as abatement; remission; diminishing of symptoms or making the injury, pathology or condition more tolerable to a subject, such as a patient in need of treatment; slowing in the rate of degeneration or decline; making the final point of degeneration less debilitating; improving a subject's physical or mental well-being. The treatment or amelioration of symptoms can be based on objective or subjective parameters; including the results of a physical examination, neuropsychiatric exams, and/or a psychiatric evaluation.

An “effective amount” is an amount sufficient to accomplish a stated purpose (e.g., achieve the effect for which it is administered, treat a disease, reduce enzyme activity, increase enzyme activity, reduce transcriptional activity, increase transcriptional activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist (inhibitor) required to decrease the activity of an enzyme or protein (e.g., transcription factor) relative to the absence of the antagonist. An “activity increasing amount,” as used herein, refers to an amount of agonist (activator) required to increase the activity of an enzyme or protein (e.g., transcription factor) relative to the absence of the agonist. A “function disrupting amount,” as used herein, refers to the amount of antagonist (inhibitor) required to disrupt the function of an enzyme or protein (e.g., transcription factor) relative to the absence of the antagonist. A “function increasing amount,” as used herein, refers to the amount of agonist (activator) required to increase the function of an enzyme or protein (e.g., transcription factor) relative to the absence of the agonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “inhibition,” “inhibit,” “inhibiting,” and grammatically equivalent variations in reference to a compound, oligomer, and/or polymer inhibiting aggregation means disrupting, preventing, or otherwise negatively affecting (e.g., decreasing) the ability of a given species, such as one or more oligo- or poly-peptides (e.g., proteins), to bind together (e.g., noncovalently) or otherwise associate relative to the level of association of such species in the absence of the compound, oligomer, and/or polymer. As defined herein, the term “inhibition”, “inhibit”, “inhibiting” and the like in reference to a protein-inhibitor (e.g., antagonist) interaction means negatively affecting (e.g., decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a signal transduction pathway or signaling pathway. Thus, inhibition includes, at least in part, partially or totally blocking stimulation, decreasing, preventing, or delaying activation, or inactivating, desensitizing, or down-regulating signal transduction or enzymatic activity or the amount of a protein.

As defined herein, the term “activation”, “activate”, “activating” and the like in reference to a protein-activator (e.g., agonist) interaction means positively affecting (e.g., increasing) the activity or function of the protein.

The term “modulator” refers to a composition that increases or decreases the level of a target molecule or the function of a target molecule.

“Patient”, “subject”, or “subject in need thereof” refers to a living organism suffering from or prone to a disease or condition that can be treated by administration of a compound or pharmaceutical composition, as provided herein. Non-limiting examples include humans, other mammals, bovines, rats, mice, dogs, monkeys, goat, sheep, cows, deer, and other non-mammalian animals. In some embodiments, a patient is human. In some embodiments, a patient is a mammal. In some embodiments, a patient is a mouse. In some embodiments, a patient is an experimental animal. In some embodiments, a patient is a rat. In some embodiments, a patient is a test animal.

“Pharmaceutically acceptable excipient” and “pharmaceutically acceptable cater” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful in the present invention.

The term “preparation” is intended to include the formulation of the active compound with encapsulating material as a cater providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). In embodiments, administration includes direct administration to a tumor. Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g., anti-cancer agent or chemotherapeutic). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another embodiment, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

As used herein, the term “conjugated” when referring to two moieties means the two moieties are bonded, wherein the bond or bonds connecting the two moieties may be covalent or non-covalent. In embodiments, the two moieties are covalently bonded to each other (e.g., directly or through a covalently bonded intermediary). In embodiments, the two moieties are non-covalently bonded (e.g., through ionic bond(s), van der waal's bond(s)/interactions, hydrogen bond(s), polar bond(s), or combinations or mixtures thereof).

As used herein, the term “about” means a range of values including the specified value, which a person of ordinary skill in the art would consider reasonably similar to the specified value. In some aspects, about means within a standard deviation using measurements generally acceptable in the art. In some aspects, about means a range extending to +/−10% of the specified value. In embodiments, about means the specified value.

As used herein, “or” is to be given its broadest reasonable interpretation, and is not to be limited to an either/or construction. Thus, the phrase “comprising A or B” means that A can be present and not B, or that B is present and not A, or that A and B are both present. Further, if A, for example, defines a class that can have multiple members, e.g., A₁ and A₂, then one or more members of the class can be present concurrently.

As used herein, “therapeutic agent” as used herein refers to a class of agents capable of treating or managing a disease, illness, or other condition of a subject. In some embodiments, the therapeutic agent is a pharmaceutical or biological agent or component or fragment thereof. In an embodiment, the therapeutic agent is a therapeutic peptide. In embodiments, the therapeutic agent may be a therapeutic peptide having a chain length of 3 to 150 amino acids, optionally of 3 to 100 amino acids, optionally 5 to 50 amino acids and optionally 5 to 20 amino acids. The therapeutic peptide may be a naturally-occurring peptide, a synthetic peptide, or a purified recombinant peptide. In some aspects, the therapeutic peptide is a naturally-occurring fusion peptide or a synthetic fusion peptide. In embodiments, the therapeutic peptide may be an agonist (activator) or an antagonist (inhibitor) of enzymatic activity or function, protein activity or function, gene expression, or a combination thereof. In some embodiments, the therapeutic peptide is a reversible antagonist or a reversible agonist. In some embodiments, the therapeutic peptide is an irreversible antagonist or an irreversible agonist. In embodiments, the therapeutic peptide may comprise a sequence derived from Htt protein, or a modified sequence thereof. In other embodiments, the therapeutic agent may be a small molecule therapeutic. In examples, the small molecule therapeutic comprises a low molecular weight organic compound having a size less than or equal to 20 nm, optionally less than or equal to 15 nm, optionally less than or equal to 10 nm. In some embodiments, the therapeutic peptide is characterized by an average molecular weight less than or equal to 40 kDa, optionally less than or equal to 30 kDa, optionally less than or equal to 20 kDa, optionally less than or equal to 10 kDa, and optionally less than or equal to 5 kDa. In some embodiments, the therapeutic peptide is characterized by an average molecular weight of 0.5 kDa to 20 kDa, optionally of 0.5 kDa to 10 kDa and optionally of 1 kDa to 5 kDa. In some aspects, at least a portion of a therapeutic peptide is or comprises the therapeutic payload of the polymers or pharmaceutical compositions described herein.

Various polymers disclosed herein are characterized, in part, by the relative amounts of distinct functional side chains present in the polymer. In aspects, the relative amounts of distinct functional side chains is represented as an average ratio defined herein as “peptide ratio.” In some embodiments, the peptide ratio is recited as a “P¹:P² ratio.” Since the degree of polymerization can vary from polymer to polymer, the composition of monomers (and functional side chains of said monomers) can also vary. Therefore, the peptide ratio should be understood as an average. It will be appreciated by one having skill in the art that polymerization methods and subsequent analysis methods are subject to random, experimental error, and the peptide ratios should therefore be read to encompass reasonable variations from the stated value. Specifically, in some aspects, peptide ratios associated with functional side chains of polymers include variations of 20% of the stated ratio. In keeping with this aspect, a P¹:P² ratio of 2:1 includes variations of P¹:P² ratios of 1.6:1 to 2.4:1 (e.g., 1.6:1, 1.8:1, 2:1, 2.2:1, 2.4:1) and 2:0.8 to 2:1.2 (e.g., 2:0.8, 2:0.9, 2:1, 2:1.1, 2:1.2). In some aspects, peptide ratios associated with functional side chains of polymers include variations of 10% of the stated ratio. In some aspects, peptide ratios associated with functional side chains of polymers include variations of ±5% of the stated ratio. In some aspects, peptide ratios associated with functional side chains of polymers include variations of ±1% of the stated ratio.

As used herein, the term “acceleration,” “accelerate,” “accelerating,” and grammatically equivalent variations in reference to a compound, oligomer, and/or polymer accelerating aggregation means promoting, facilitating, or otherwise positively affecting (e.g., increasing or speeding up) the ability of a given species, such as one or more oligo- or poly-peptides (e.g., proteins), to bind together (e.g., noncovalently) or otherwise associate relative to the level of association of such species in the absence of the compound, oligomer, and/or polymer. Acceleration of aggregation is relevant, for example, in the context of speeding up aggregation past a toxic species, such as nanofibirils, towards higher order aggregates that are less toxic, so as to reduce overall toxicity of the system.

As used herein, the term “bind,” “binding,” and grammatically equivalent variations in reference to a compound, oligomer, and/or polymer binding to a given species, such as one or more oligo- or poly-peptides (e.g., proteins), means that the compound, oligomer, and/or polymer makes multiple noncovalent bonds to the given species. In some aspects, the multiple noncovalent bonds are possible because the compound, oligomer, and/or polymer has a portion with a similar and/or corresponding amino acid sequence to a portion of the given species.

As used herein, the phrase “at least a portion of each instance of P¹ independently comprises at least” a specified percentage of amino acid composition similarity and/or sequence homology, and similar phrasing, refers to a discrete segment of P¹ having the indicated amino acid composition similarity and/or sequence homology. For clarity, the specified percentage is not determined by references to single amino acids taken from unrelated segments of P¹.

As used herein, the phrase “at least a portion of each instance of P¹ independently is or comprises at least one of” specified amino acid sequences, and similar phrasing, means that each P¹ can be any of the specified amino acid sequences or any combination of the specified amino acid sequences.

As used herein, “metaphilic” means a compound, oligomer, or polymer that is transiently amphiphilic, such as by way of a hydrophobic backbone with hydrophilic side chains. In some aspect, the metaphilicity leads to globular but fluxional structures, which may be useful for penetrating cell walls.

As used herein, “polyQ disease” or “polyglutamine disease” refers to a group of neurodegenerative diseases or disorders characterized by polyglutamine expansion mutations, wherein CAG (i.e., cytosine, adenine, guanine) trinucleotide repeats are translated into expanded polyglutamine regions in certain proteins. PolyQ diseases include, at least, Huntington's disease (HD), different types of spinocerebellar ataxias (SCAs), Alzheimer's disease, and Parkinson's disease.

As used herein, “HV-3,” “HV3,” “HV-3 peptide,” or “HV3 peptide” refers to a peptide capable of inhibiting the protein-protein interaction between VCP and mtHtt. In some embodiments, the HV3 peptide refers to SEQ ID NO: 5, which is discussed in detail in Guo, X. et al. VCP Recruitment to Mitochondria Causes Mitophagy Impairment and Neurodegeneration in Models of Huntington's Disease. Nat. Commun. 2016, 7. Unless otherwise indicated, “HV3” does not refer to an HV3 peptide conjugated to an HIV-derived TAT peptide. In preferred embodiments, the HV3 peptide is a modified HV3 peptide. In keeping with this aspect, in some embodiments, the modified HV3 peptide comprises a modification to delete or replace one or more, optionally all, instances of cysteine residues. In some embodiments, the cysteine residue is replaced with a polar, uncharged amino acid such as serine, threonine, asparagine, or glutamine. For example, the cysteine residue may be replaced with a serine residue. In some embodiments, the modified HV3 peptide comprises a modification to increase cellular uptake. For example, the modification may comprise the addition of one or more (e.g., one, two, three, four, five, or six) positively charged amino acid residues, such as an arginine or a lysine residue. In preferred embodiments, the modified HV3 peptide comprises or consists of SEQ ID NO: 1, SEQ ID NO:2, SEQ ID NO: 3, or SEQ ID: 4.

Other terms are defined in other portions of this description, even though not included in this subsection.

### DETAILED DESCRIPTION

In the following description, numerous specific details of the polymers, polymer components, peptides, and methods of the present invention are set forth in order to provide a thorough explanation of the precise nature of the invention. It will be apparent, however, to those of skill in the art that the invention can be practiced without these specific details.

In contrast to the classical, biological, linear polypeptide configuration, the present invention employs a new class of “polypeptide” wherein peptides form side chains originating from a polymer backbone scaffold. These structures, termed protein-like polymers (PLPs), are synthesized via graft-through, living polymerization methods using peptide-modified monomers, for example as discussed in Callmann, C. E.; Thompson, M. P.; Gianneschi, N. C. Poly(Peptide): Synthesis, Structure, and Function of Peptide-Polymer Amphiphiles and Protein-like Polymers. Acc. Chem. Res. 2020, 53 (2), 400-413, which is incorporated by reference herein in its entirety, and more specifically for methodologies of making PLPs and discussions of structure and function, to the extent not inconsistent with the description herein. The resulting structure, consisting of a hydrophobic polymer, with dense array of hydrophilic side-chain peptides leads to the formation of a globular “protein-like” structure protecting the peptides from proteolytic degradation and providing a means to penetrate cells, with judicious choice of charge, combined with their metaphilic, or transiently amphiphilic structure.

It is believed that PLPs, having a higher molecular weight compared to free peptides, avoid rapid renal clearance upon systemic injection. The PLP platform is highly versatile and modular due to synthetic control over the displayed peptides and/or small molecules and the wide range of available peptides and peptide modifications. Herein, the utility of the PLP system to overcome the challenges facing peptide-based therapeutics is disclosed, using the HV3 peptide displayed on a PLP backbone as a proof-of-concept in a HD mouse model. The PLP is shown to penetrate mouse-derived HD striatal neurons (HdhQ111) containing homozygous HTT loci with polyglutamine repeats, successfully blocking VCP/mtHtt binding, an intracellular protein-protein interaction (PPI). This PPI serves as a proof-of-concept target, providing further encouragement to pursue other intracellular disease driving and difficult to drug protein interfaces. Further, they demonstrate biocompatibility, low toxicity, and exceptional resistance to enzymatic proteolysis, and show enhanced therapeutic efficacy over the native peptide.

The inventive polymer can be any suitable polymer type described herein and can comprise, or be derived from, any suitable number of monomers. For example, in some embodiments, the polymer is a homopolymer (i.e., derived from/incorporating one type of monomer). Alternatively, in some embodiments, the polymer can be a copolymer comprising (e.g., derived from/incorporating) more than one type of monomer (e.g., from 2 to 10 types of monomers). It will be understood that the inventive polymer, along with the linked polymer side chains, can have any suitable configuration. For example, in some embodiments wherein the polymer is a homopolymer, the polymer can be a brush polymer. In other embodiments wherein the polymer is a copolymer, the polymer can be a brush block copolymer or brush random/statistical copolymer.

In some aspects, the polymer is characterized by a P¹ peptide density selected from the range 50% to 100%, optionally some embodiments a density selected from the range of 75% to 100%, or optionally for some embodiments a density selected from the range of 90% to 100%. In aspects, the polymer is characterized by a P² peptide density selected from the range 50% to 100%, optionally some embodiments a density selected from the range of 75% to 100%, or optionally for some embodiments a density selected from the range of 90% to 100%.

In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to a sequence found in a HV-3 peptide or a modified HV-3 peptide. In aspects, the peptide of the functional side chain group comprises any suitable number of amino acid units so long as the peptide comprises a sequence having 75% or greater (e.g., 80% or greater, 85% or greater, 90% or greater, 95% or greater, or 100%) sequence identity to one or more of SEQ ID: 1-5. In some aspects, the peptide comprises at least 5 amino acid residues. For example, the peptide comprises 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, 27 or more, 28 or more, 29 or more, 30 or more, 31 or more, 32 or more, or 33 or more amino acid units. Alternatively, or in addition, the peptide can comprise 100 or less amino acid units, for example, 90 or less, 80 or less, 70 or less, 60 or less, 59 or less, 58 or less, 57 or less, 56 or less, 55 or less, 54 or less, 53 or less, 52 or less, 51 or less, 50 or less, 49 or less, 48 or less, 47 or less, 46 or less, 45 or less, 44 or less, 43 or less, 42 or less, 41 or less, 40 or less amino acid units. Thus, the peptide can comprise a number of amino acid units bounded by any two of the aforementioned endpoints. For example, the peptide can comprise 5 to 100 amino acid units, for example, 5 to 100, 5 to 90, 5 to 80, 5 to 70, 5 to 60, 5 to 50, 5 to 40, 5 to 30, 5 to 20, 5 to 16, 5 to 15, 5 to 14, 6 to 100, 6 to 90, 6 to 80, 6 to 70, 6 to 60, 6 to 50, 6 to 40, 6 to 30, 6 to 20, 6 to 16, 6 to 15, 6 to 14, 7 to 100, 7 to 90, 7 to 80, 7 to 70, 7 to 60, 7 to 50, 7 to 40, 7 to 30, 7 to 20, 7 to 16, 7 to 15, 7 to 14, 8 to 100, 10 to 100, 10 to 90, 10 to 80, 10 to 70, 10 to 60, 10 to 50, 10 to 40, 10 to 30, 10 to 20, 10 to 16, 10 to 15, 10 to 14, 11 to 16, 11 to 15, 11 to 14, 12 to 20, 12 to 16, 12 to 15, or 12 to 14 amino acid units. In some embodiments, the peptide comprises 6 to 20 amino acid residues. In certain embodiments, the peptide comprises 6 to 16 amino acid residues.

The functional side group comprising a peptide can have any suitable structure (e.g., primary, secondary, tertiary, or quaternary structure) described herein. The peptide can be a branched peptide, a linear peptide, cyclic peptide, or a cross-linked peptide. In some embodiments, the polymer is characterized by a structure wherein at least a portion of the peptide is linked to the polymer backbone group via an enzymatically degradable linker, such a matrix metalloproteinase (MMP) cleavage sequence, cathepsin B cleavage sequence, ester bond, reductive sensitive bond-disulfide bond, pH sensitive bond-imine bond or any combinations of these. In other embodiments, the polymer is characterized by a structure wherein at least a portion of the peptide side-chain is linked to the polymer backbone or consists of a degradable or triggerable linker.

In some aspects, the peptide comprises a sequence having hydrophobic regions, such as leucine-rich regions. In aspects, the hydrophobic regions may be modified to substitute hydrophilic amino acid residues or non-hydrophobic amino acid residues. It is believed that such substitutions may facilitate improved solubility of the polymer if necessary for certain applications. In aspects where superior stability is desired, the polymer may also be modified. Acceptable polymer modifications include asparagine β-hydroxylation, higher degrees of polymerization (e.g., greater than 10 DP, greater than 15 DP, greater than 30 DP, or greater than 45 DP), single point mutations, and other suitable modifications.

Additionally, the peptide may comprise one or more gaps in its sequence. For example, the peptide may comprise 5 consecutive amino acid residues which do not impact, or do not contribute to, the properties of the peptide's sequence. In aspects, the one or more gaps is a spacer molecule, 5 or less amino acid residues, 3 or less amino acid residues, or a combination thereof.

In some specific embodiments, the polymer comprises a tag for imaging and/or analysis. In aspects, the polymer comprises a fluorescein-, biotin-, or rhodamine-based tag resulting in a fluorescently labelled PLP. For example, each polymer segment B¹, B², or B³ of formula (FX1) or (FX2) can independently comprise a tag for imaging and/or analysis. Similarly, each P¹, P², or R¹ of formula (FX1), (FX2), and (RU1)-(RU3c) can independently comprise a tag for imaging and/or analysis. Additionally, each T¹ or T² of any of the formulas described herein can independently comprise a tag for imaging and/or analysis. For example, the polymer can comprise one or more of a dye, a radiolabeling agent, an imaging agent, titration agent, and the like.

In embodiments, L¹ and L² are linking groups, and optionally a linking group comprising a polymer grafting group. In some embodiments, L¹ and L² independently are optionally functionalized by one or more additional substituents, such as peptide substituents or substituents derived from small molecules. In some embodiments, L¹ and L² independently comprise a single bond, an oxygen, and groups having an alkylene group, a heteroalkylene group, an alkenylene group, an arylene group, an alkoxy group, an acyl group, a triazole group, a diazole group, a pyrazole group, and combinations thereof. In some embodiments, L¹ and L² independently comprise a linking group selected from the group consisting of a single bond, —O—, —(CH₂CH₂O)ₓ—, C₁-C₁₀ alkyl, C₁-C₁₀ acyl, C₂-C₁₀ alkenyl, C₃-C₁₀ aryl, C₁-C₁₀ alkoxyl, or any combination thereof, wherein x is an integer from 1 to 20.

The inventive polymers can have any suitable degrees of polymerization. If the degree of polymerization is too low, the polymer may not be resistant to adhesive force or may not be resistant to enzymatic cleavage by proteases or may be cleared too rapidly from the body since the polymer's molecular weight would be lower than the clearance threshold through the kidney. Additionally, if too low, the polymer may exhibit poor solubility and structural instability. Alternatively, if the degree of polymerization is too high, the peptide side chain groups displayed on the polymer may be too dense to engage their biological targets such as cell receptors, enzymes, PPIs etc. Additionally, the high degree of polymerization may result in a polymer that is too large to penetrate cells. Typically, the polymer has a degree of polymerization of 2 to 1000 (e.g., 2 to 500, 2 to 250, 2 to 100, 2 to 60, 2 to 50, 2 to 30, 5 to 1000, 5 to 500, 5 to 250, 5 to 100, 5 to 60, 5 to 50, 5 to 45, 5 to 30, 7 to 45, 20 to 500, 20 to 250, 20 to 100, 20 to 50, or 20 to 30). In certain embodiments, the polymer has a degree of polymerization of 5 to 100. In preferred embodiments, the polymer has a degree of polymerization of 7 to 30. For example, the polymer can have a degree of polymerization of 2 or about 2, 5 or about 5, a degree of polymerization of 10 or about 10 (e.g., 11), a degree of polymerization of 15 or about 15 (e.g., 17), a degree of polymerization of 20 or about 20, a degree of polymerization of 30 or about 30, a degree of polymerization of 50 or about 50, a degree of polymerization of 60 or about 60, a degree of polymerization of 100 or about 100, a degree of polymerization of 150 or about 150, or a degree of polymerization of 200 or about 200. In some embodiments, the polymer has a degree of polymerization of 2 to 50. In certain embodiments, the polymer has a degree of polymerization of at least 5. In other certain embodiments, the polymer has a degree of polymerization of at least 7.

Additionally, in aspects the polymer comprises a brush density greater than or equal to 50% (e.g., greater than or equal to 60%, greater than or equal to 65%, greater than or equal to 70%, greater than or equal to 75%, greater than or equal to 80%, greater than or equal to 85%, or greater than or equal to 90%), optionally for some embodiments a density greater than or equal to 70%, or optionally for some embodiments a density greater than or equal to 90%. Brush polymers of certain aspects have a brush density selected from the range 50% to 100%, optionally some embodiments a density selected from the range of 75% to 100%, or optionally for some embodiments a density selected from the range of 90% to 100%. Brush polymers of preferred aspects have a brush density selected from the range 75% to 100%. Brush polymers of certain aspects have a “high brush density” selected from the range 90% to 100%, optionally some embodiments a density selected from the range of 95% to 100%, or optionally for some embodiments a density selected from the range of 99% to 100%. For example, in aspects of the invention, the polymer may be characterized by a formulation wherein 90% of its polymer segments comprise a polymer backbone group covalently linked to a functional side chain group, wherein each polymer segment may comprise the same or different functional side chain group. In aspects, said functional side chain group comprises a peptide which comprises a sequence having 75% or greater sequence identity of a sequence found in a Huntingtin protein, such as a portion of the HV-3 region. In some embodiments, the brush density of the polymer is equal to the peptide density of a particular peptide (e.g., all polymer segments of the polymer comprising polymer backbones covalently linked to a polymer side chain comprising P¹). In other aspects, the brush density of the polymer is different from the peptide density of a particular peptide (e.g., at least one polymer segment comprises a polymer backbone covalently linked to a polymer side chain comprising P¹ and at least one other polymer segment comprises a polymer backbone covalently linked to a polymer side chain comprising P² wherein P¹ and P² are characterized by different sequences).

Additionally, for each of the polymers characterized by the formula (FX1) or (FX2) and/or (RU1)-(RU3c) described herein, it will be understood that the first repeating unit

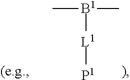

the second repeating unit

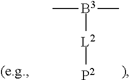

when present, and a third repeating unit

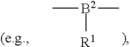

when present, can be arranged in any suitable order. For example, the first repeating unit, the second repeating unit, and the third repeating unit can be arranged as a random polymer, block polymer, brush, brush block, alternating, segmented, grafted, tapered and other architectures. In other words, variables “m”, “n”, and “o” merely define the total number of that particular monomer in the polymer and do not imply any particular order. For each of the polymers characterized by the formula (FX1) or (FX2), each of m, n, and o can be independently selected from any suitable integer. Suitability of the integer is based on the desired DP for each repeating unit as discussed herein.

The polymer backbone groups units (e.g., each of B¹, B², and B³ of the polymers characterized by the formula (FX1) and (FX2), and the backbone group units of (RU1)-(RU3c)), can be independently selected from any suitable polymer backbone subunit. In aspects, each of the first polymer backbone subunit, the second polymer backbone subunit, and the third polymer backbone subunit can be a monomer capable of undergoing ring opening metathesis. For example, each of B¹, B², and B³ can independently be a substituted or unsubstituted norbornene, oxanorbornene, olefin, cyclic olefin, cyclooctene, or cyclopentadiene. In some aspects, each of the first polymer backbone group subunit, the second polymer backbone group subunit, and/or the third polymer backbone group subunit is a polymerized norbornene dicarboxyimide monomer. In some embodiments, each polymer backbone subunit of the polymer is a polymerized norbornene dicarboxyimide monomer. In aspects where the polymer has poor solubility, one or more of the polymer backbone subunits may be substituted with an oxanorbornene-based subunit (if not already in use) or other suitable hydrophilic backbone subunit.

Preferably, in any embodiment of a polymer, a method, a use, a composition, or a medicament disclosed herein, the polymer is stable against enzymatic digestion. Optionally in any embodiment of a polymer, a method, a use, a composition, or a medicament disclosed herein, the polymer is stable against enzymatic digestion by a metalloproteinase. Optionally in any embodiment of a polymer, a method, a use, a composition, or a medicament disclosed herein, the polymer is stable against enzymatic digestion by matrix metalloproteinase and thermolysin. Preferably in any embodiment of a polymer, a method, a use, a composition, or a medicament disclosed herein, the polymer is stable against enzymatic digestion for at least 450 minutes. Optionally in any embodiment a polymer, a method, a use, a composition, or a medicament disclosed herein, each polymer individually solvated by water when a plurality of said polymers is dispersed in water.

In another aspect, the invention provides a pharmaceutical composition comprising one or more peptides and/or one or more polymers described herein. In some embodiments, the composition comprises one or more pharmaceutically acceptable excipients. For example, the peptides and/or polymers of the invention can be formulated for parenteral administration, such as intravenous (IV) administration or administration into a body cavity or lumen of an organ. Alternatively, the peptides and/or polymers can be injected intra-tumorally. Formulations for injection will commonly comprise a solution of the peptide and/or polymer dissolved in a pharmaceutically acceptable carrier. Among the acceptable vehicles and solvents that can be employed are water and an isotonic sodium chloride. In addition, sterile fixed oils can conventionally be employed as a solvent or suspending medium. For this purpose, any bland fixed oil can be employed including synthetic monoglycerides or diglycerides. In addition, fatty acids such as oleic acid can likewise be used in the preparation of injectables. These solutions are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the peptide and/or polymer in these formulations can vary widely, and will be selected primarily based on fluid volumes, viscosities, body weight, and the like, in accordance with the particular mode of administration selected and the patient's needs. In certain embodiments, the concentration of a peptide and/or polymer in a solution formulation for injection will range from 0.1% (w/w) to 10% (w/w) or about 0.1% (w/w) to about 10% (w/w).

The present invention further provides methods for using the inventive polymers disclosed herein. In aspects, the inventive polymers may be used as a therapeutic, a PPI disrupting agent, mitophagy inhibiting agent, or any combination thereof. In some embodiments, the methods described herein can be used to treat or manage a neurodegenerative disease or condition, such as a polyQ disease. In some aspects, the polyQ disease is Huntington's disease (HD). The method includes administering a therapeutically effective amount of a polymer or a peptide described herein and a pharmaceutically acceptable excipient to a subject, a cell, or a tissue in need thereof. In some aspects, the polymers or peptides disclosed herein are formulated as a pharmaceutical composition. In some aspects, the pharmaceutical composition contains a pharmaceutically acceptable carrier, e.g. phosphate buffered saline solution, mixtures of ethanol in water, water and emulsions such as an oil/water or water/oil emulsion, as well as various wetting agents or excipients. In some aspects, the pharmaceutical composition also contains excipients and/or carriers such as solvents, dispersants, coatings, absorption promoting agents, controlled release agents, and inert excipients, such as starches, granulating agents, microcrystalline cellulose, diluents, lubricants, binders, and disintegrating agents. In some aspects, the pharmaceutical composition is formulated as a soluble powder, a liquid concentrate, or a ready-to-use formulation. In some aspects, the proper dosage of the pharmaceutical composition is determined in a conventional manner, based upon factors such as the subject's condition, immune status, body weight and age. For example, the amount of polymer or peptide required to be administered for treating or managing a neurodegenerative disease or condition in a subject will vary depending upon factors such as the risk and severity of the underlying condition(s), any other medical conditions or diseases, age, the form of the composition, and other medications being administered. Further the amount may vary depending upon whether the polymer or peptide is being used to treat (when the dose may be higher) or whether the polymer or peptide is being used as a secondary prevention/maintenance (when the dose may be lower). However, the required amount can be readily set by a medical practitioner. For example, the methods can include administering the polymer to provide a dose of from 10 ng/kg to 50 mg/kg to the subject. For example, the polymer dose can range from 1 mg/kg to 50 mg/kg, from 10 μg/kg to 5 mg/kg, or from 100 μg/kg to 4 mg/kg. In some embodiments, the polymer dose is 3 mg/kg with respect to peptide. The polymer dose can also lie outside of these ranges, depending on the particular polymer as well as the type of disease being treated. Frequency of administration can range from a single dose to multiple doses per week, or more frequently. In some embodiments, the polymer is administered from about once per month to about five times per week. In some embodiments, the polymer is administered once per week.

The present invention further provides a method for making the inventive polymers disclosed herein. In aspects of the method for making the inventive polymer, the at least one peptide is capped at the terminal end with any suitable polymerizable monomer. In aspects, the polymerizable monomer may comprise an ethylenically unsaturated monomer. In aspects, the polymerizable monomer may comprise an olefin-based functional group, a norbornene-amide hexanoic acid, (meth)acrylate, or a norbornene dicarboxyamide. The polymerizable monomers may be polymerized by ROMP, RAFT, or ATRP, aspects of which are further described in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51(9), 1861-1881, each of which is incorporated by reference herein in its entirety, and more specifically for methodologies of making a polymer, to the extent not inconsistent with the description herein.

After polymerization the inventive polymers may be characterized using any suitable technique(s). Typically, the inventive polymers are characterized by size-exclusion chromatography with multiangle light scattering (SEC-MALS), sometimes referred to as gel permeation chromatography (GPC), to ascertain degree of polymerization (DP) and molecular weight distribution (dispersity or Mw/Mn). Alternatively, or in addition to, the inventive polymers may be characterized by SDS-PAGE to ascertain degree of polymerization (DP) and molecular weight. Preferably, there is suitable agreement between the obtained DP and the theoretical DP based on the initial monomer-to-initiator ratio ([M]₀/[I]₀).

The polymer can be administered by oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. In some embodiments, the polymer is administered intravenously, subcutaneously, intramuscularly, topically, orally, or a combination thereof. In some embodiments, the polymer is administered to a subject's brain, spinal cord, cerebrospinal fluid, or any combination thereof. The methods described herein can comprise contacting a target tissue of the subject with the polymer or a metabolite or product thereof, contacting a target cell of the subject with the polymer or a metabolite or product thereof, and/or contacting a target receptor of the subject with the polymer or a metabolite or product thereof, and/or contacting a target peptide of the subject with the polymer or a metabolite or product thereof. In embodiments, the polymers described herein pass through the cell membrane and contact an intracellular target. Without wishing to be bound by any particular theory, it is believed that the PLP structure and charge described herein play an integral role in providing cell permeability.

### Aspects of the Invention

Various aspects are contemplated herein, several of which are set forth in the paragraphs below. It is explicitly contemplated that any aspect or portion thereof can be combined to form an aspect. In addition, it is explicitly contemplated that any aspect (e.g., Aspect A13) that references an aspect (e.g., Aspect A1) for which there are sub-aspects having the same top level number (e.g., Aspect A1a, A1b, A1c, and so forth) necessarily includes reference to those sub-aspects A1a, A1b, A1c, and so forth. Furthermore, although the aspects below are subdivided into aspects A, B, C, D, and so forth, it is explicitly contemplated that aspects in each of subdivisions A, B, C, D, etc. can be combined in any manner. Moreover, the term “any preceding aspect” means any aspect that appears prior to the aspect that contains such phrase (in other words, the sentence “Aspect B13: The method of any one of aspects B1-B12, or any preceding aspect, . . . ” means that any aspect prior to aspect B13 is referenced, including aspects B1-B12 and all of the “A” aspects). For example, it is contemplated that, optionally, any method or composition of any of the below aspects may be useful with or combined with any other aspect provided below. Further, for example, it is contemplated that any embodiment described elsewhere herein, including above this paragraph, may optionally be combined with any of the below listed aspects. In some instances in the aspects below, or elsewhere herein, two open ended ranges are disclosed to be combinable into a range. For example, “at least X” is disclosed to be combinable with “less than Y” to form a range, in which X and Y are numeric values. For the purposes of forming ranges herein, it is explicitly contemplated that “at least X” combined with “less than Y” forms a range of X-Y inclusive of value X and value Y, even though “less than Y” in isolation does not include Y.

Aspect A1: A polymer comprising a first repeating unit comprising a first polymer backbone subunit directly or indirectly covalently linked to a first functional sidechain comprising a peptide having 75% or greater (e.g., 75% or greater, 80% or greater, 85% or greater, 90% or greater, 95% or greater, or 100%) sequence identity of a homologous region between VCP and Htt.

Aspect A2: The polymer of aspect A1, wherein the homologous region between VCP and Htt comprises a sequence having 75% or greater (e.g., 75% or greater, 80% or greater, 85% or greater, 90% or greater, 95% or greater, or 100%) sequence identity of an HV3 peptide.

Aspect A3: The polymer of aspect A1 or aspect A2, further comprising a second repeating unit comprising a second polymer backbone subunit directly or indirectly covalently linked to a second functional sidechain comprising a second peptide that is different from the peptide of the first functional sidechain.

Aspect B1: A polymer characterized by a formula (FX1):

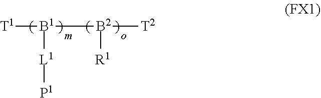

wherein:
each P¹ independently comprises a peptide;
at least one P¹ independently, or in combination with other instances of P¹, comprises an HV3 peptide or a modified HV3 peptide;
T¹ and T² are each independently polymer backbone terminating groups that can be the same or different;
B¹ and B² are each independently a polymer backbone subunit;
L¹ is optionally present and is a linking group;
R¹ is independently a substituent;
m is an integer from 2 to 1000 (e.g., 2 to 1000, 2 to 500, 2 to 100, 2 to 50, 2 to 30, 2 to 20, 5 to 1000, 5 to 500, 5 to 250, 5 to 100, 5 to 50, 5 to 30, 5 to 20, 7 to 1000, 7 to 500, 7 to 250, 7 to 100, 7 to 50, 7 to 30, 20 to 500, 20 to 250, 20 to 100, 20 to 50, 20 to 30);
o is an integer from 0 to 1000 (e.g., 0 to 500, 0 to 250, 0 to 100, 0 to 50, 0 to 30, 0 to 20, 2 to 1000, 2 to 500, 2 to 250, 2 to 100, 2 to 50, 2 to 30, 0 to 20, 5 to 1000, 5 to 500, 5 to 250, 5 to 100, 5 to 50, 5 to 30, 5 to 20, 7 to 1000, 7 to 500, 7 to 250, 7 to 100, 7 to 50, 7 to 30, 20 to 500, 20 to 250, 20 to 100, 20 to 50, or 20 to 30);
each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms;
each instance of B¹, B², L¹, R¹, and P¹ is the same as or different from any other instance of B¹, B², L¹, R¹, and P¹, respectively; and
when o is an integer from 1 to 1000 and/or at least one instance of P¹ is different from another instance of P¹, the polymer is a block copolymer or a statistical copolymer.

Aspect B2: The polymer of aspect B1, or any preceding aspect, wherein the polymer is characterized by a formula (FX2):

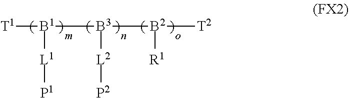

wherein:
T¹ and T² are each independently polymer backbone terminating groups that can be the same or different;
B¹, B², and B³ are each independently polymer backbone subunits;
each L¹ and L² is optionally present and each is independently a linking group;
each P¹ and P² independently comprise a peptide or a small molecule;
at least one P¹ independently, or in combination with other instances of P¹, comprises an HV3 peptide or a modified HV3 peptide;
each instance of P² is different from each instance of P¹;
each R¹ is independently a substituent;
m is an integer selected from the range of 2 to 1000 (e.g., 2 to 1000, 2 to 500, 2 to 100, 2 to 50, 2 to 30, 2 to 20, 5 to 1000, 5 to 500, 5 to 250, 5 to 100, 5 to 50, 5 to 30, 5 to 20, 7 to 1000, 7 to 500, 7 to 250, 7 to 100, 7 to 50, 7 to 30, 20 to 500, 20 to 250, 20 to 100, 20 to 50, 20 to 30);
n is an integer selected from the range of 1 to 1000 (e.g., 1 to 1000, 1 to 500, 1 to 100, 1 to 50, 1 to 30, 1 to 20, 2 to 1000, 2 to 500, 2 to 100, 2 to 50, 2 to 30, 5 to 1000, 5 to 500, 5 to 250, 5 to 100, 5 to 50, 5 to 30, 5 to 20, 7 to 1000, 7 to 500, 7 to 250, 7 to 100, 7 to 50, 7 to 30, 20 to 500, 20 to 250, 20 to 100, 20 to 50, 20 to 30);
o is an integer selected from the range of 0 to 1000 (e.g., 0 to 500, 0 to 250, 0 to 100, 0 to 50, 0 to 30, 0 to 20, 2 to 1000, 2 to 500, 2 to 250, 2 to 100, 2 to 50, 2 to 30, 0 to 20, 5 to 1000, 5 to 500, 5 to 250, 5 to 100, 5 to 50, 5 to 30, 5 to 20, 7 to 1000, 7 to 500, 7 to 250, 7 to 100, 7 to 50, 7 to 30, 20 to 500, 20 to 250, 20 to 100, 20 to 50, or 20 to 30);
each connecting line in formula (FX2) represents a covalent linkage comprising at least one of a single bond, a double bond, one or more atoms, or any combination thereof, optionally, for example, each connecting line represents a single bond or double bond;
each instance of B¹, B², B³, L¹, L², R¹, P¹, and P² is the same as or different from any other instance of B¹, B², B³, L¹, L², R¹, P¹, and P², respectively; and the polymer is a block copolymer or a statistical copolymer.

Aspect B3: The polymer of aspect B1 or B2, or any preceding aspect, wherein at least one (or optionally all) of B¹, B², or B³ comprises a polymerized monomer comprising an unsaturated monomer.

Aspect B4: The polymer of aspect B3, or any preceding aspect, wherein the unsaturated monomer comprises an ethylenically unsaturated monomer, a norbornene monomer, or a norbornene dicarboxyimide.

Aspect B5: The polymer of any one aspects B1-B4, or any preceding aspect, wherein each instance of a repeating unit (RU1) and (RU2):

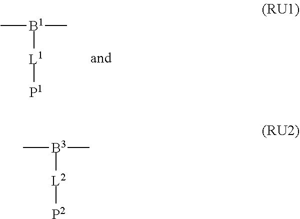

in formula (FX1) or (FX2) is independently characterized by a repeating unit (RU3), (RU4), (RU5), or (RU6):

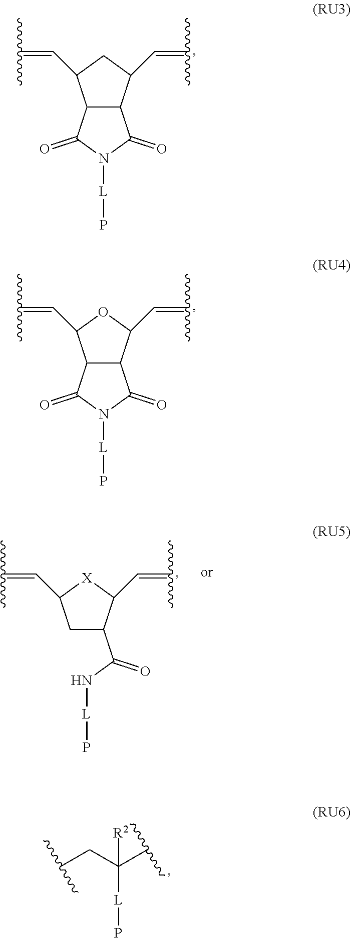

wherein:
L is optionally present and is L¹ or L²;
P is P¹ or P²;
R² is H or C₁-C₃ alkyl; and
X is CH₂ or O.

Aspect B6: The polymer of aspect B5, or any preceding aspect, wherein each instance of the repeating unit (RU3):

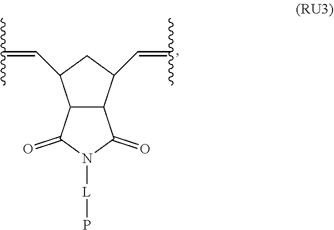

in formula (FX1) or (FX2) is independently characterized by a repeating unit (RU3a), (RU3b), or (RU3c):

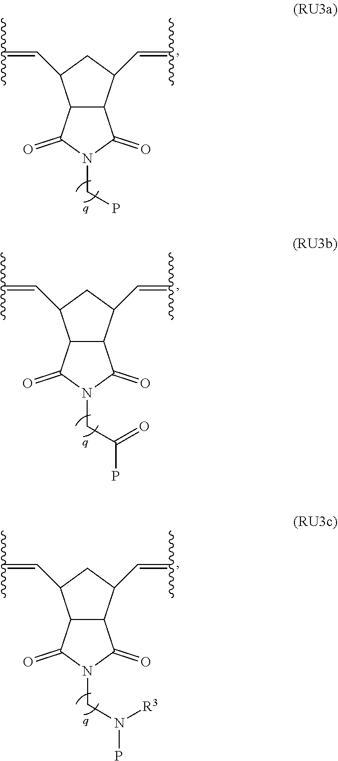

wherein q is an integer from 1 to 20 (e.g., 1 to 20, 1 to 15, 1 to 10, or 1 to 5) and R³ is a hydrogen or a C₁-C₅ alkyl.

Aspect B7: The polymer of any one of aspects B1-B6, or any preceding aspect, wherein each instance of L¹ and L² is independently selected from a single bond, an oxygen, and groups having an alkylene group, a heteroalkylene group, an alkenylene group, an arylene group, an alkoxy group, an acyl group, a triazole group, a diazole group, a pyrazole group, and combinations thereof.

Aspect B8: The polymer of any one of aspects B1-B7, or any preceding aspect, wherein each instance of L¹ and L² is independently selected from a single bond, —O—, C₁-C₁₀ alkyl, C₂-C₁₀ alkylene, C₁-C₁₀ heteroalkylene, C₃-C₁₀ arylene, C₁-C₁₀ alkoxy, C₁-C₁₀ acyl and combinations thereof.

Aspect B9: The polymer of any one of aspects B1-B8, or any preceding aspect, wherein each instance of L¹ and L² is independently selected from —(CH₂)ₙNR—, —(CH₂)ₙC(O)NR—, —(CH₂)ₙNRC(O)—, —(CH₂)ₙC(O)— and —(CH₂)ₙ—, wherein n is an integer from 1 to 20 and R is hydrogen or a C₁-C₅ alkyl.

Aspect B10: The polymer of any one of aspects B1-B9, or any preceding aspect, wherein each instance of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R⁴, —CONR⁵R⁶, —COR⁷, —SOR⁸, —OSR⁹, —SO₂R¹⁰, —OR¹¹, —SR¹², —NR¹³R¹⁴, —NR¹⁵COR¹⁶, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R⁴-R¹⁶ independently is H, C₅-C₁₀ aryl, or C₁-C₁₀ alkyl.

Aspect B11: The polymer of any one of aspects B1-B10, or any preceding aspect, wherein at least one of R¹, T¹, T², P¹, and P² further comprises an analytical tag.

Aspect B12: The polymer of aspect B11, or any preceding aspect, wherein the analytical tag comprises an affinity tag, a solubilization tag, a chromatography tag, an epitope tag, a contrast agent tag, or a fluorescence tag.

Aspect B13: The polymer of any one of aspects A1-B12, wherein the polymer is characterized by a number average molecular weight of 1 kDa to 100 kDa (e.g., from 1 kDa to 100 kDa, from 1 kDa to 50 kDa, from 1 kDa to 30 kDa, from 5 kDa to 100 kDa, from 5 kDa to 50 kDa, from 5 kDa to 30 kDa, from 7 kDa to 100 kDa, from 7 kDa to 50 kDa, from 7 kDa to 30 kDa, from 10 kDa to 100 kDa, from 10 kDa to 50 kDa, or from 10 kDa to 30 kDa).

Aspect B14: The polymer of any one of aspects A1-B13, wherein the polymer is characterized by a number average molecular weight of 1 kDa to 50 kDa (e.g., from 1 kDa to 50 kDa, from 1 kDa to 30 kDa, from 5 kDa to 50 kDa, from 5 kDa to 30 kDa, from 7 kDa to 50 kDa, from 7 kDa to 30 kDa, from 10 kDa to 50 kDa, or from 10 kDa to 30 kDa).

Aspect B15: The polymer of any one of aspects A1-B14, wherein the polymer is characterized by an average degree of polymerization of 2 to 1000 (e.g., 2 to 1000, 2 to 500, 2 to 200, 2 to 100, 2 to 80, 2 to 60, 2 to 50, 2 to 30, 2 to 20, 7 to 1000, 7 to 500, 7 to 200, 7 to 100, 7 to 80, 7 to 60, 7 to 40, 7 to 30, 7 to 20, 15 to 50, 15 to 40, or 15 to 30).

Aspect B16: The polymer of any one of aspects A1-B15, wherein the polymer is characterized by an average degree of polymerization of 2 to 100 (e.g., 2 to 100, 2 to 80, 2 to 60, 2 to 50, 2 to 30, 2 to 20, 7 to 100, 7 to 80, 7 to 60, 7 to 40, 7 to 30, 7 to 20, 15 to 50, 15 to 40, or 15 to 30).

Aspect B17: The polymer of any one of aspects A1-B16, wherein the polymer is characterized by an average degree of polymerization of 2 to 30 (e.g., 2 to 30, 2 to 20, 5 to 30, 5 to 20, 7 to 30, 7 to 20, 15 to 40, or 15 to 30).

Aspect B18: The polymer of any one of aspects A1-B17, wherein the polymer is prepared by a living polymerization method optionally selected from ring-opening metathesis polymerization (ROMP), reversible addition-fragmentation chain transfer polymerization (RAFT), or atom transfer radical polymerization (ATRP).

Aspect B19: The polymer of any one of aspects A1-B18, wherein the polymer is a high-density brush polymer characterized by a brush density greater than or equal to 75% (e.g., greater than or equal to 75%, greater than or equal to 80%, greater than or equal to 85%, greater than or equal to 90%, greater than or equal to 95%, or 100%).

Aspect B20: The polymer of any one of aspects A1-B19, wherein the polymer is a high-density brush polymer characterized by a brush density greater than or equal to 85% (greater than or equal to 85%, greater than or equal to 90%, greater than or equal to 95%, or 100%).

Aspect B21: The polymer of any one of aspects A1-B20, wherein the polymer is metaphilic.

Aspect B22: The polymer of any one of aspects A1-B21, wherein at least one functional sidechain, optionally all functional sidechains, comprises gaps between one or more amino acid residues, wherein the gaps comprise up to 15 (e.g., up to 10, up to 5) amino acid residues or other spacer molecules.

Aspect B23: The polymer of any one of aspects A1-B22, wherein the polymer is characterized by a P¹ peptide density of greater than or equal to 85% (e.g., greater than or equal to 85%, greater than or equal to 90%, greater than or equal to 95%, or 100%).

Aspect B24: The polymer of any one of one of aspects A1-B23, wherein the polymer is characterized by a P¹ peptide density of greater than or equal to 95% (e.g., 99% or 100%).

Aspect B25: The polymer of any one of aspects B1-B24, or any preceding aspect, wherein each instance of P¹ and P² independently comprises from 3 to 50 amino acid residues (e.g., 3 to 50, 3 to 40, 3 to 30, 3 to 20, 5 to 50, 5 to 40, 5 to 30, 5 to 20, 6 to 50, 6 to 40, 6 to 30, or 6 to 20 amino acid residues).

Aspect B25a: The polymer of any one of aspects B1-B25, or any preceding aspect, wherein each instance of P¹ and P² independently comprises from 3 to 30 amino acid residues.

Aspect B26: The polymer of any one of aspects B1-B25a, or any preceding aspect, wherein at least 75% (e.g., at least 75%, at least 80%, at least 85%, at least 90%, at least 95%, or 100%) of all instances of P¹ on a number basis comprises an amino acid sequence derived from Htt.

Aspect B27: The polymer of any one of aspects B1-B26, or any preceding aspect, wherein at least 75% (e.g., at least 75%, at least 80%, at least 85%, at least 90%, at least 95%, or 100%) of all instances of P¹ on a number basis comprises a sequence identity having 75% or greater (e.g., 75% or greater, 80% or greater, 85% or greater, 90% or greater, 95% or greater, or 100%) sequence identity of an HV3 peptide or a modified HV3 peptide.

Aspect B27a: The polymer of any one of aspects B1-B26, or any preceding aspect, wherein at least 75% (e.g., at least 75%, at least 80%, at least 85%, at least 90%, at least 95%, or 100%) of all instances of P¹ on a number basis comprises a sequence identity having 75% or greater (e.g., 75% or greater, 80% or greater, 85% or greater, 90% or greater, 95% or greater, or 100%) sequence identity of a modified HV3 peptide.

Aspect B28: The polymer of any one of aspects B1-B27a, or any preceding aspect, wherein at least one, optionally each instance of, P¹ comprises a protein-protein interaction inhibitor.

Aspect B29: The polymer of aspect B28, or any preceding aspect, wherein the protein-protein interaction comprises the interaction of VCP with mtHtt.

Aspect B30: The polymer of any one of aspects B1-B29, or any preceding aspect, wherein at least one, optionally each instance, of P¹ or P² is characterized by a net positive charge.

Aspect B31: The polymer of any one of aspects B1-B30, or any preceding aspect, wherein at least one of P¹ or P² further comprises a charge modulating domain.

Aspect B32: The polymer of aspect B31, or any preceding aspect, wherein the charge modulating domain is a cationic residue domain.

Aspect B33: The polymer of aspect B32, or any preceding aspect, wherein the cationic residue domain consists of lysine, arginine, histidine, asparagine, or a combination thereof.

Aspect B34: The polymer of any one of aspects B1-B33, or any preceding aspect, wherein at least 75% (e.g., 75% or greater, 80% or greater, 85% or greater, 90% or greater, 95% or greater, or 100%) of all instances of P¹ on a number basis are characterized by a net positive charge.

Aspect B35: The polymer of any one of aspects B1-B34, or any preceding aspect, wherein the polymer has a P¹:P² ratio of between 1:1 and 15:1. For example, a P¹:P² ratio of 1:1, 2:1, 3:1, 4:1, 5:1, 6:1, 8:1, 10:1, 12:1, or 15:1.

Aspect B36: The polymer of any one of aspects B1-B35, or any preceding aspect, wherein the polymer has a P¹:P² ratio of between 1:1 and 5:1. For example, a P¹:P² ratio of 1:1, 2:1, 3:1, 4:1, or 5:1.

Aspect B37: The polymer of any one of aspects B1-B36, or any preceding aspect, wherein at least one, optionally each instance of, P¹ comprises or consists of a sequence having 75% or greater (e.g., 75% or greater, 80% or greater, 85% or greater, 90% or greater, 95% or greater, or 100%) sequence identity of SEQ ID NO: 1 (HVLVMSATRR).

Aspect B37a: The polymer of any one of aspects B1-B37, or any preceding aspect, wherein at least one, optionally each instance of, P¹ comprises or consists of a sequence having 90% or greater (e.g., 90% or greater, 95% or greater, or 100%) sequence identity of SEQ ID NO: 1 (HVLVMSATRR).

Aspect B37b: The polymer of any one of aspects B1-B37a, or any preceding aspect, wherein at least one, optionally each instance of, P¹ comprises or consists of a sequence having 95% or greater (e.g., 95% or greater, 99% or greater, or 100%) sequence identity of SEQ ID NO: 1 (HVLVMSATRR).

Aspect B38: The polymer of any one of aspects B1-B37b, or any preceding aspect, wherein at least one, optionally each instance of, P¹ comprises or consists of a sequence having a sequence identity of SEQ ID NO: 1 (HVLVMSATRR).

Aspect B39: The polymer of any one of aspects B1-B38, or any preceding aspect, wherein at least one, optionally each instance of, P¹ comprises or consists of a sequence having 75% or greater (e.g., 75% or greater, 80% or greater, 85% or greater, 90% or greater, 95% or greater, or 100%) sequence identity of SEQ ID NO: 2 (HVLVMSATKK).

Aspect B39a: The polymer of any one of aspects B1-B39, or any preceding aspect, wherein at least one, optionally each instance of, P¹ comprises or consists of a sequence having 90% or greater (e.g., 90% or greater, 95% or greater, or 100%) sequence identity of SEQ ID NO: 2 (HVLVMSATKK).

Aspect B39b: The polymer of any one of aspects B1-B39a, or any preceding aspect, wherein at least one, optionally each instance of, P¹ comprises or consists of a sequence having 95% or greater (e.g., 95% or greater, 99% or greater, or 100%) sequence identity of SEQ ID NO: 2 (HVLVMSATKK).

Aspect B40: The polymer of any one of aspects B1-B39b, or any preceding aspect, wherein at least one, optionally each instance of, P¹ comprises or consists of a sequence having a sequence identity of SEQ ID NO: 2 (HVLVMSATKK).

Aspect B41: The polymer of any one of aspects B1-B40, or any preceding aspect, wherein at least one, optionally each instance of, P¹ comprises or consists of a sequence having 75% or greater (e.g., 75% or greater, 80% or greater, 85% or greater, 90% or greater, 95% or greater, or 100%) sequence identity of SEQ ID NO: 3 (HVLVMSATRRRR).

Aspect B41a: The polymer of any one of aspects B1-B41, or any preceding aspect, wherein at least one, optionally each instance of, P¹ comprises or consists of a sequence having 90% or greater (e.g., 90% or greater, 95% or greater, or 100%) sequence identity of SEQ ID NO: 3 (HVLVMSATRRRR).

Aspect B41b: The polymer of any one of aspects B1-B41a, or any preceding aspect, wherein at least one, optionally each instance of, P¹ comprises or consists of a sequence having 95% or greater (e.g., 95% or greater, 99% or greater, or 100%) sequence identity of SEQ ID NO: 3 (HVLVMSATRRRR).

Aspect B42: The polymer of any one of aspects B1-B41b, or any preceding aspect, wherein at least one, optionally each instance of, P¹ comprises or consists of a sequence having a sequence identity of SEQ ID NO: 3 (HVLVMSATRRRR).

Aspect B43: The polymer of any one of aspects B1-B42, or any preceding aspect, wherein at least one, optionally each instance of, P¹ comprises or consists of a sequence having 75% or greater (e.g., 75% or greater, 80% or greater, 85% or greater, 90% or greater, 95% or greater, or 100%) sequence identity of SEQ ID NO: 4 (HVLVMSATKKKK).

Aspect B43a: The polymer of any one of aspects B1-B43, or any preceding aspect, wherein at least one, optionally each instance of, P¹ comprises or consists of a sequence having 90% or greater (e.g., 90% or greater, 95% or greater, or 100%) sequence identity of SEQ ID NO: 4 (HVLVMSATKKKK).

Aspect B43b: The polymer of any one of aspects B1-B43a, or any preceding aspect, wherein at least one, optionally each instance of, P¹ comprises or consists of a sequence having 95% or greater (e.g., 95% or greater, 99% or greater, or 100%) sequence identity of SEQ ID NO: 4 (HVLVMSATKKKK).

Aspect B44: The polymer of any one of aspects B1-B43b, or any preceding aspect, wherein at least one, optionally each instance of, P¹ comprises or consists of a sequence having a sequence identity of SEQ ID NO: 4 (HVLVMSATKKKK).

Aspect B45: The polymer of any one of aspects B1-B44, or any preceding aspect, wherein at least 75% (e.g., 75% or greater, 80% or greater, 85% or greater, 90% or greater, 95% or greater, or 100%) of all instances of P¹ on a number basis comprise:

SEQ ID NO: 1
(HVLVMSATRR);
SEQ ID NO: 2
(HVLVMSATKK);
SEQ ID NO: 3
(HVLVMSATRRRR);
SEQ ID NO: 4
(HVLVMSATKKKK);
SEQ ID NO: 9
(HVLVMSAT);

or
any combination thereof.

Aspect B46: The polymer of any one of aspects B1-B45, or any preceding aspect, wherein at least 75% (e.g., 75% or greater, 80% or greater, 85% or greater, 90% or greater, 95% or greater, or 100%) of all instances of P¹ on a number basis comprise:

SEQ ID NO: 1
(HVLVMSATRR);
SEQ ID NO: 4
(HVLVMSATKKKK);

or
any combination thereof.

Aspect B47: The polymer of any one of aspects B1-B46, or any preceding aspect, wherein at least one P¹ comprises a sequence having 75% or greater (e.g., 75% or greater, 80% or greater, 85% or greater, 90% or greater, 95% or greater, or 100%) sequence identity of SEQ ID NO: 5 (HVLVMCAT).

Aspect B47a: The polymer of any one of aspects B1-B47, or any preceding aspect, wherein at least one, optionally each instance of, P¹ comprises or consists of a sequence having 90% or greater (e.g., 90% or greater, 95% or greater, or 100%) sequence identity of SEQ ID NO: 5 (HVLVMCAT).

Aspect B47b: The polymer of any one of aspects B1-B47a, or any preceding aspect, wherein at least one, optionally each instance of, P¹ comprises or consists of a sequence having 95% or greater (e.g., 95% or greater, 99% or greater, or 100%) sequence identity of SEQ ID NO: 5 (HVLVMCAT).

Aspect B48: The polymer of any one of aspects B1-B47b, or any preceding aspect, wherein at least one, optionally each instance of, P² comprises a sequence having 75% or greater (e.g., 75% or greater, 80% or greater, 85% or greater, 90% or greater, 95% or greater, or 100%) sequence identity of SEQ ID NO: 6 (AASSGVSTPGSAGHDIITEQPRS).

Aspect B48a: The polymer of any one of aspects B1-B48, or any preceding aspect, wherein at least one, optionally each instance of, P² comprises or consists of a sequence having 90% or greater (e.g., 90% or greater, 95% or greater, or 100%) sequence identity of SEQ ID NO: 6 (AASSGVSTPGSAGHDIITEQPRS).

Aspect B48b: The polymer of any one of aspects B1-B48a, or any preceding aspect, wherein at least one, optionally each instance of, P² comprises or consists of a sequence having 95% or greater (e.g., 95% or greater, 99% or greater, or 100%) sequence identity of SEQ ID NO: 6 (AASSGVSTPGSAGHDIITEQPRS).

Aspect B49: The polymer of any one of aspects B1-B48b, or any preceding aspect, wherein at least one, optionally each instance of, P² comprises a sequence having 75% or greater (e.g., 75% or greater, 80% or greater, 85% or greater, 90% or greater, 95% or greater, or 100%) sequence identity of SEQ ID NO: 7 (SNWKWWPGIFD).

Aspect B49a: The polymer of any one of aspects B1-B49, or any preceding aspect, wherein at least one, optionally each instance of, P² comprises or consists of a sequence having 90% or greater (e.g., 90% or greater, 95% or greater, or 100%) sequence identity of SEQ ID NO: 7 (SNWKWWPGIFD).

Aspect B49b: The polymer of any one of aspects B1-B49a, or any preceding aspect, wherein at least one, optionally each instance of, P¹ comprises or consists of a sequence having 95% or greater (e.g., 95% or greater, 99% or greater, or 100%) sequence identity of SEQ ID NO: 7 (SNWKWWPGIFD).

Aspect B50: The polymer of any one of aspects B1-B49b, or any preceding aspect, wherein at least one, optionally each instance of, P² comprises a sequence having 75% or greater (e.g., 75% or greater, 80% or greater, 85% or greater, 90% or greater, 95% or greater, or 100%) sequence identity of SEQ ID NO: 8 (GRKKRRQRRRPPQSSEIVLDGTDN).

Aspect B50a: The polymer of any one of aspects B1-B50, or any preceding aspect, wherein at least one, optionally each instance of, P² comprises or consists of a sequence having 90% or greater (e.g., 90% or greater, 95% or greater, or 100%) sequence identity of SEQ ID NO: 8 (GRKKRRQRRRPPQSSEIVLDGTDN).

Aspect B50b: The polymer of any one of aspects B1-B50a, or any preceding aspect, wherein at least one, optionally each instance of, P¹ comprises or consists of a sequence having 95% or greater (e.g., 95% or greater, 99% or greater, or 100%) sequence identity of SEQ ID NO: 8 (GRKKRRQRRRPPQSSEIVLDGTDN).

Aspect B51: The polymer of any one of aspects B1-B50b, or any preceding aspect, wherein at least 85% (e.g., at least 85%, at least 90%, at least 95%, or 100%) of all instances of P¹ on a number basis do not comprise sequences having cysteine residues.

Aspect B52: The polymer of any one of aspects B1-B51, or any preceding aspect, wherein at least one, optionally each instance, of P¹ or P² comprises or further comprises a point mutation or substitution to comprise at least one arginine, at least one lysine, or a combination thereof.

Aspect B53: The polymer of any one of aspects B1-B52, or any preceding aspect, wherein at least one of P¹ and P² comprises or further comprises a point mutation or substitution to comprise at least one serine.

Aspect B53a: The polymer of any one of aspects B1-B52, or any preceding aspect, wherein at least one of P¹ and P² comprises or further comprises a point mutation or substitution to comprise at least one serine, threonine, asparagine, or glutamine.

Aspect B54: The polymer of any one of aspects B1-B53a, or any preceding aspect, wherein the polymer has at least one of the following properties:
(a) P¹ comprises 5-100 amino acids (e.g., 5 to 100, 5 to 50, 5 to 40, 5 to 30, 5 to 20, 6 to 50, 6 to 100, 6 to 40, 6 to 30, or 6 to 20 amino acids);
(b) m is an integer from 2 to 100 (e.g., 2 to 100, 2 to 80, 2 to 60, 2 to 50, 2 to 30, 2 to 20, 2 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(c) n is an integer from 0 to 100 (e.g., 0 to 100, 0 to 80, 0 to 60, 0 to 50, 0 to 30, 0 to 20, 0 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(d) o is an integer from 0 to 100 (e.g., 0 to 100, 0 to 80, 0 to 60, 0 to 50, 0 to 30, 0 to 20, 0 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30);
(d) m is an integer from 2 to 100 (e.g., 2 to 100, 2 to 80, 2 to 60, 2 to 50, 2 to 30, 2 to 20, 2 to 10, 4 to 100, 4 to 80, 4 to 60, 4 to 50, 4 to 30, 4 to 20, 4 to 10, 10 to 50, 10 to 40, 10 to 30, 20 to 50, 20 to 40, or 20 to 30), n is 0, p is 0, and at least one instance of P¹ is different from another instance of P¹;
(e) degree polymerization (m+n+o) is an integer from 2 to 200 (e.g., 2 to 200, 2 to 100, 2 to 80, 2 to 60, 2 to 50, 2 to 30, 2 to 20, 2 to 10, 4 to 200, 4 to 100, 4 to 80, 4 to 60, 4 to 50, 4 to 30, 4 to 20, 4 to 10, 10 to 200, 10 to 50, 10 to 40, 10 to 30, 20 to 50, 20 to 40, or 20 to 30), or 2 to 50 (e.g., 2 to 50, 2 to 30, 2 to 20, 2 to 10, 4 to 50, 4 to 30, 4 to 20, 4 to 10, 10 to 50, 10 to 40, 10 to 30, 20 to 50, 20 to 40, or 20 to 30);
(f) molecular weight of from 1 kDa to 1,000 kDa (e.g., from 1 kDa to 1000 kDa, from 1 kDa to 500 kDa, from 1 kDa to 100 kDa, from 1 kDa to 50 kDa, from 1 kDa to 30 kDa, from 5 kDa to 50 kDa, from 5 kDa to 30 kDa, from 7 kDa to 1000 kDa, from 7 kDa to 50 kDa, from 7 kDa to 30 kDa, from 10 kDa to 1000 kDa, from 10 kDa to 50 kDa, or from 10 kDa to 30 kDa);
(g) the polymer has a P¹ peptide density of at least 50% (e.g., at least 50%, at least 55%, at least 60%, at least 65%, at least 70%, at least 75%, at least 80%, at least 85%, at least 90%, at least 95%, or at least 99%), as defined by equation m/(m+n+o)×100;
(h) a combination thereof,
(i) any combination thereof.

Aspect C1: A composition comprising the polymer of any one of aspects A1-B54 and a pharmaceutically acceptable excipient.

Aspect C2: The composition of aspect C1, or any preceding aspect, wherein the pharmaceutical composition comprises two or more of the polymers of aspects A1-B54.

Aspect D1: A method for selectively inhibiting the mitochondrial autophagy pathway in a cell, the method comprising:
contacting the cell with an effective amount of the polymer of any one of aspects A1-B54 or the pharmaceutical composition of aspect C1 or C2;
wherein the contacting results in the selective inhibition of the mitochondrial autophagy pathway in the cell.

Aspect E1: A method for selectively inhibiting the interaction between VCP and mtHtt in a cell, the method comprising:
contacting the cell with an effective amount of the polymer of any one of aspects A1-B54 or the pharmaceutical composition of aspect C1 or C2;
wherein the contacting results in the selective inhibition of the interaction between VCP and mtHtt in the cell.

Aspect E2: The method of aspect E1, or any preceding aspect, wherein the contacting results in the selective inhibition of the interaction between VCP and mtHtt in or near the mitochondria of the cell.

Aspect F1: A method for preventing or treating a neurodegenerative disease or condition in a subject, the method comprising:
administering to the subject a therapeutically effective amount of the polymer of any one of aspects A1-B54 or the composition of aspect C1 or C2;
thereby preventing or treating the neurodegenerative disease or condition in the subject.

Aspect F2: The method of aspect F2, or any preceding aspect, further comprising:
repeating the step of administering to the subject the therapeutically effective amount of the polymer of any one of aspects A1-B54 or the pharmaceutical composition of aspect C1 or C2.

Aspect F3: The method of aspect F1 or F2, or any preceding aspect, wherein the neurodegenerative disease or condition comprises a polyglutamine disease.

Aspect F4: The method of any one of aspects F1-F3, or any preceding aspect, wherein the neurodegenerative disease or condition comprises Huntington's disease.

Aspect F5: The method of any one of aspects F1-F4, or any preceding aspect, wherein the polymer or composition is administered to the subject's brain, spinal cord, cerebrospinal fluid, or any combination thereof.

Aspect G1: A method of targeting a mitochondrial localized protein in a cell comprising:
introducing the polymer of any one of aspects A1-B54 or the composition of aspect C1 or C2 to the cell;
wherein the introducing results in at least a portion of the polymer to bind with at least a portion of the mitochondrial localized protein;
thereby targeting a mitochondrial localized protein in a cell.

Aspect G2: The method of aspect G1, or any preceding aspect, wherein the mitochondrial localized protein is a mtHtt protein.

Aspect H1: A method of making the polymer of any one of aspects A1-B54, the method comprising:
synthesizing the at least one P¹ peptide;
capping the at least one P¹ peptide at a terminal end with a polymerizable monomer that, once polymerized, becomes polymer backbone subunit B¹, thereby forming a polymerizable P¹ monomer;
polymerizing the polymerizable P¹ monomer.

Aspect H2: The method of aspect H1, or any preceding aspect, wherein:
the synthesizing step comprises solid-phase synthesis using protected amino acids;
the polymerizable monomer comprises an ethylenically unsaturated monomer optionally comprising norbornene or a (meth)acrylate; and
the polymerizing step comprises ROMP, RAFT, or ATRP.

Aspect I1: A therapeutic peptide having 75% or greater (e.g., 75% or greater, 80% or greater, 85% or greater, 90% or greater, 95% or greater, or 100%) sequence identity of SEQ ID NO: 1.

Aspect I1a: A therapeutic peptide having 90% or greater (e.g., 90% or greater, 95% or greater, or 100%) sequence identity of SEQ ID NO: 1.

Aspect I1b: A therapeutic peptide having 95% or greater (e.g., 95% or greater, 99% or greater, or 100%) sequence identity of SEQ ID NO: 1.

Aspect I2: The therapeutic peptide of any one of aspects I1-I1b, or any preceding aspect, wherein the therapeutic peptide has a sequence identity of SEQ ID NO: 1.

Aspect J1: A therapeutic peptide having 75% or greater (e.g., 75% or greater, 80% or greater, 85% or greater, 90% or greater, 95% or greater, or 100%) sequence identity of SEQ ID NO: 2.

Aspect J1a: A therapeutic peptide having 90% or greater (e.g., 90% or greater, 95% or greater, or 100%) sequence identity of SEQ ID NO: 2.

Aspect J1b: A therapeutic peptide having 95% or greater (e.g., 95% or greater, 99% or greater, or 100%) sequence identity of SEQ ID NO: 2.

Aspect J2: The therapeutic peptide of any one of aspects J1-J1b, or any preceding aspect, wherein the therapeutic peptide has a sequence identity of SEQ ID NO: 2.

Aspect K1: A therapeutic peptide having 75% or greater (e.g., 75% or greater, 80% or greater, 85% or greater, 90% or greater, 95% or greater, or 100%) sequence identity of SEQ ID NO: 3.

Aspect K1a: A therapeutic peptide having 90% or greater (e.g., 90% or greater, 95% or greater, or 100%) sequence identity of SEQ ID NO: 3.

Aspect K1b: A therapeutic peptide having 95% or greater (e.g., 95% or greater, 99% or greater, or 100%) sequence identity of SEQ ID NO: 3.

Aspect K2: The therapeutic peptide of any one of aspects K1-K1b, or any preceding aspect, wherein the therapeutic peptide has a sequence identity of SEQ ID NO: 3.

Aspect L1: A therapeutic peptide having 75% or greater (e.g., 75% or greater, 80% or greater, 85% or greater, 90% or greater, 95% or greater, or 100%) sequence identity of SEQ ID NO: 4.

Aspect L1a: A therapeutic peptide having 90% or greater (e.g., 90% or greater, 95% or greater, or 100%) sequence identity of SEQ ID NO: 4.

Aspect L1b: A therapeutic peptide having 95% or greater (e.g., 95% or greater, 99% or greater, or 100%) sequence identity of SEQ ID NO: 4.

Aspect L2: The therapeutic peptide of any one of aspects L1-L1b, or any preceding aspect, wherein the therapeutic peptide has a sequence identity of SEQ ID NO: 4.

Aspect M1: A pharmaceutical composition comprising the therapeutic peptide of any one of aspects I1-L2, or any preceding aspect, and a pharmaceutically acceptable excipient.

Aspect M2: The pharmaceutical composition of aspect M1, or any preceding aspect, wherein the pharmaceutical composition comprises two or more of the therapeutic peptides of aspects I1-L2.

Aspect N1: A method for selectively inhibiting the mitochondrial autophagy pathway in a cell, the method comprising:
contacting the cell with an effective amount of the therapeutic peptide of any one of aspects I1-L2 or the pharmaceutical composition of aspect M1 or M2;
wherein the contacting results in the selective inhibition of the mitochondrial autophagy pathway in the cell.

Aspect O1: A method for selectively inhibiting the interaction between VCP and mtHtt in a cell, the method comprising:
contacting the cell with an effective amount of the therapeutic peptide of any one of aspects I1-L2 or the pharmaceutical composition of aspect M1 or M2;
wherein the contacting results in the selective inhibition of the interaction between VCP and mtHtt in the cell.

Aspect O2: The method of aspect O1, or any preceding aspect, wherein the contacting results in the selective inhibition of the interaction between VCP and mtHtt in or near the mitochondria of the cell.

Aspect P1: A method for preventing or treating a neurodegenerative disease or condition in a subject, the method comprising:
administering to the subject a therapeutically effective amount of the therapeutic peptide of any one of aspects I1-L2 or the pharmaceutical composition of aspect M1 or M2;
thereby preventing or treating the neurodegenerative disease or condition in the subject.

Aspect P2: The method of aspect P2, or any preceding aspect, further comprising:
repeating the step of administering to the subject the therapeutically effective amount of the therapeutic peptide of any one of aspects I1-L2 or the pharmaceutical composition of aspect M1 or M2.

Aspect P3: The method of aspect P1 or P2, or any preceding aspect, wherein the neurodegenerative disease or condition comprises a polyglutamine disease.

Aspect P4: The method of any one of aspects P1-P3, or any preceding aspect, wherein the neurodegenerative disease or condition comprises Huntington's disease.

Aspect P5: The method of any one of aspects P1-P4, or any preceding aspect, wherein the therapeutic peptide or pharmaceutical composition is administered to the subject's brain, spinal cord, cerebrospinal fluid, or any combination thereof.

Aspect Q1: A method of targeting a mitochondrial localized protein in a cell comprising: introducing the therapeutic peptide of any one of aspects I1-L2 or the pharmaceutical composition of aspect M1 or M2 to the cell;
wherein the introducing results in at least a portion of the therapeutic peptide to bind with at least a portion of the mitochondrial localized protein;
thereby targeting a mitochondrial localized protein in a cell.

Aspect Q2: The method of aspect Q1, or any preceding aspect, wherein the mitochondrial localized protein is a mtHtt protein.

### EXAMPLES

The invention can be further understood by the following non-limiting examples. The examples are provided to illustrate some of the concepts described within this disclosure. While each example is considered to provide specific individual embodiments of composition and methods of preparation and use, none of the examples should be considered to limit the more general embodiments described herein.

In the following examples, efforts have been made to ensure accuracy with respect to numbers used (e.g., amounts, temperature, etc.) but some experimental error and deviation should be accounted for.

Materials and Methods: The following descriptions of materials and methods describe the materials and methods used in one or more of the below Examples, as applicable. To the extent of any conflict between the materials and methods of these descriptions and those provided in a particular Example, the Example controls.

Materials: All materials and reagents, unless otherwise noted, were purchased from Sigma Aldrich and Fisher Chemicals. All amino acids used to prepare peptides by solid-phase peptide synthesis (SPPS) were obtained from AAPPTec, Chem-Impex, and NovaBiochem. N-(hexanoic acid)-cis-5-norbomene-exo-dicarboximide was synthesized according to methods discussed in Patel, P. R. et al. Synthesis and cell adhesive properties of linear and cyclic RGD functionalized polynorbornene thin films. Biomacromolecules 2012, 13 (8), 2546-53, which is incorporated herein by reference in its entirety, and specifically for methods of synthesis, to the extent not inconsistent with the description herein. CellTiter-Blue® was purchased from Promega Corporation. Dulbecco's Phosphate Buffered Saline (DPBS) with Ca²⁺ and Mg²⁺ was purchased from Corning Cellgro. Valosin containing protein (VCP, His tag) was purchased from Antibodies-online. All the enzymes were purchased from Promega.

Instrumentation: ¹H NMR: ¹H NMR spectra were recorded on a Varian Inova spectrometer (500 MHz) in DMF-d₇; Analytical HPLC: Analytical HPLC analysis of peptides was performed on a Jupiter 4 Proteo 90A Phenomenex column (150×4.60 mm) using a Hitachi-Elite LaChrom L-2130 pump equipped with UV-Vis detector (Hitachi-Elite LaChrom L2420). The solvent system consists of (A) 0.1% TFA in water and (B) 0.1% TFA in acetonitrile; Preparative HPLC: An Armen Glider CPC preparatory HPLC was used to purify all peptides. The solvent system consists of (A) 0.1% TFA in water and (B) 0.10% TFA in acetonitrile; ESI-MS: ESI-MS spectra of peptides were collected using a Bruker Amazon-SL spectrometer configured with an ESI source in both negative and positive ionization mode; MALDI-ToF MS: MALDI-ToF MS spectra of polymers in CHCA matrix were collected using a Bruker AutoFlex III Smartbeam spectrometer in both negative and positive ionization modes; Organic GPC: Organic phase GPC measurements were performed on a set of Phenomenex Phenogel 5 m, 1K-75K, 300×7.80 mm in series with a Phenomex Phenogel 5 m, 10K-1000K, 300×7.80 mm columns with HPLC grade solvents as eluents: dimethylformamide (DMF) with 0.05 M of LiBr at 60° C. Detection consisted of a Hitachi UV-Vis Detector L-2420, a Wyatt Optilab T-rEX refractive index detector operating at 658 nm and a Wyatt DAWN® HELEOS® II light scattering detector operating at 659 nm. Absolute molecular weights and polydispersities were calculated using the Wyatt ASTRA software with dn/dc values determined by assuming 100% mass recovery during GPC analysis; Aqueous GPC: Aqueous phase GPC measurements were performed on an Agilent 1200 HPLC system with a PSS Suprema column using HPLC grade water with 0.10% trifluoroacetic acid buffer as the mobile phase. Detection consisted of a Wyatt Optilab T-rEX refractive index detector operating at 658 nm and a Wyatt DAWN® HELEOS® II light scattering detector operating at 659 nm. Absolute molecular weights and polydispersities were calculated using the Wyatt ASTRA software with dn/dc values determined by assuming 100% mass recovery during GPC analysis; Flow Cytometry: Cellular uptake of P1/HV3-TAT was analyzed via flow cytometry using a BD FacsAria IIu 4-Laser flow cytometer (Becton Dickinson Inc., USA). Mean fluorescence intensity were prepared for presentation using FlowJo v10; CLSM: Imaging was accomplished using LEICA SP5 II laser scanning confocal microscope with a 63× oil immersion objective at 1.5× optical zoom. All the images were Z-stack images. Slice thickness was 0.26 μm with a scan size of 1024×1024 pixels and a scan speed of 400 Hz. The cell nuclei (stained with Hoechst) was accomplished using a 358 nm laser with a 15% laser power. Cell imaging for the membrane (stained with wheat germ agglutinin) was accomplished using a 488 nm laser with a 12% laser power. Rhodamine-labeled P1 and HV3-TAT were imaged using a 561 nm laser with 12% laser power; Fluorescence Measurements: CellTiter-Blue® fluorescence measurements were recorded using a Perkin Elmer EnSpire multimode Plate Reader; Cell Culture: HdhQ111 cells originally purchased from the Cornell Institute. Cells were incubated at 33° C. at 5% CO2 using DMEM (high glucose, no glutamine, Life Technologies/Gibco, Cat. 11960044) supplemented with 10% FBS (heat inactivated, Omega Scientific, Cat FB-02), and 1× of sodium pyruvate (100x=100 mM, Life Technologies, Cat. 11360070), G-418 Disulfate (DOT Scientific, DSG64500), GlutaMAX (Life technologies, Cat. 35050061) and antibiotics (Penicillin-Streptomycin, Life Technologies Cat 15140122). Cell cultures were maintained by subculturing in flasks every 4-7 days when cells became confluent using trypsin-EDTA, 0.25% (Life Technologies, Cat 25200114); Animal Care: Mice (CD1, C57BL/6J, R6/2) for in vivo studies were obtained from the Jackson Laboratory. All animal procedures were approved by NU's institutional animal care and use committee (IACUC) and performed by Developmental Therapeutics Core; ICP-MS: ICP-MS was performed at the Northwestern University Quantitative Bioelemental Imaging Core on the Thermo iCAP Q ICP-MS, controlled using QTEGRA software.

PLPs: The PLPs tested in the below examples are occasionally referred to as “PX” where “X” denotes a specific peptide sequence. For example, P1 refers to a PLP generated from peptide monomers characterized by SEQ ID: 1; P2 refers to a PLP generated from peptide monomers characterized by SEQ ID: 2; P3 refers to a PLP generated from peptide monomers characterized by SEQ ID: 3; and P4 refers to a PLP generated from peptide monomers characterized by SEQ ID: 4. Similarly, monomers (i.e., monomers which have not been incorporated into the PLP platform) in the below examples are occasionally referred to as “MX” where “X” denotes a specific peptide sequence.

Peptide Synthesis: Peptide monomers are synthesized on Rink resin (0.67 mmol/g) using standard Fmoc SPPS procedures on the Liberty Blue Automated Microwave Synthesizer. Peptide monomers are prepared via amide coupling to N-(hexanoic acid)-cis-5-norbornene-exo-dicarboximide (3.0 equiv.) in the presence of HBTU (2.9 equiv.) and DIPEA (6.0 equiv.). Aspects of this process are described in detail in Blum et al., Peptides Displayed as High Density Brush Polymers Resist Proteolysis and Retain Bioactivity, J. Am. Chem. Soc. 2014, 136 (43), 15422-15437, which is incorporated by reference herein in its entirety, and more specifically for methodologies of synthesis, to the extent not inconsistent with the description herein. The peptide monomers are cleaved off the resin by treating the resin with trifluoroacetic acid (TFA):H₂O:triisopropyl silane (95:2.5:2.5 v/v) for 2 h. The crude products are obtained by precipitation in cold diethyl ether.

Peptides are further purified with a Jupiter Proteo 90 Å Phenomenex column (2050×25.0 mm) on an Armen Glider CPC preparatory phase HPLC to yield 90-95% purity, confirmed by analytical HPLC. For all RP-HPLC purifications, gradient solvent systems utilize Buffer A (water with 0.1% TFA) and Buffer B (acetonitrile with 0.1% TFA). A gradient of 15-45% buffer B over 30 min is used to purify all peptides and monomers. Pure products are then analyzed by ESI-MS.

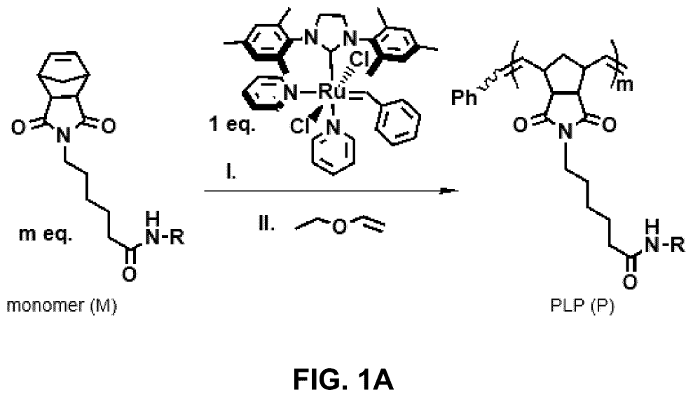

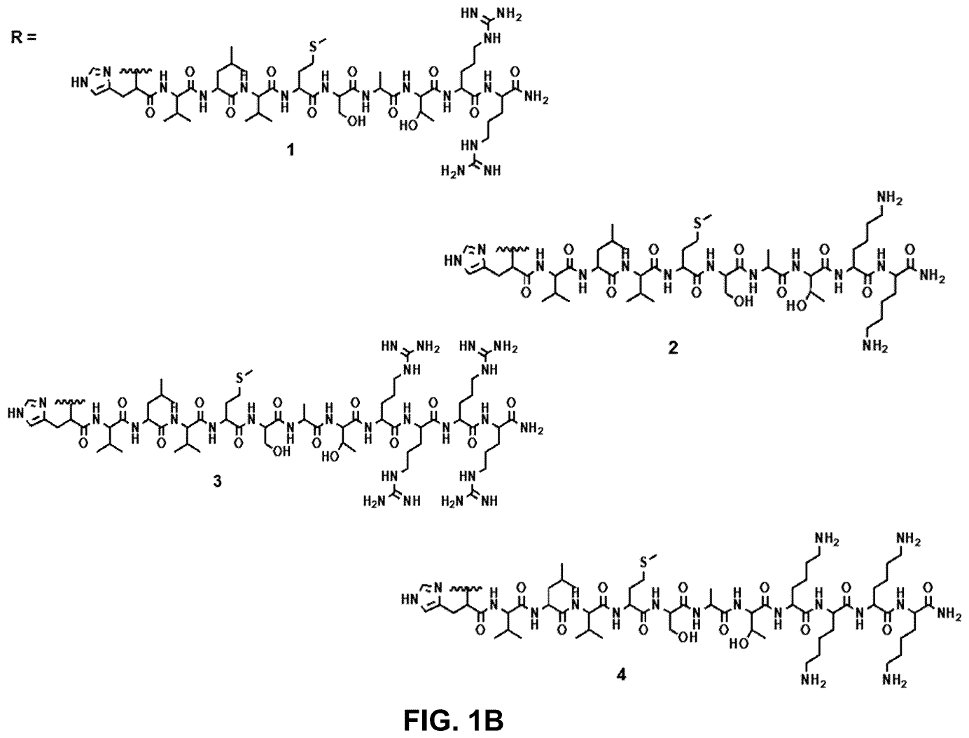

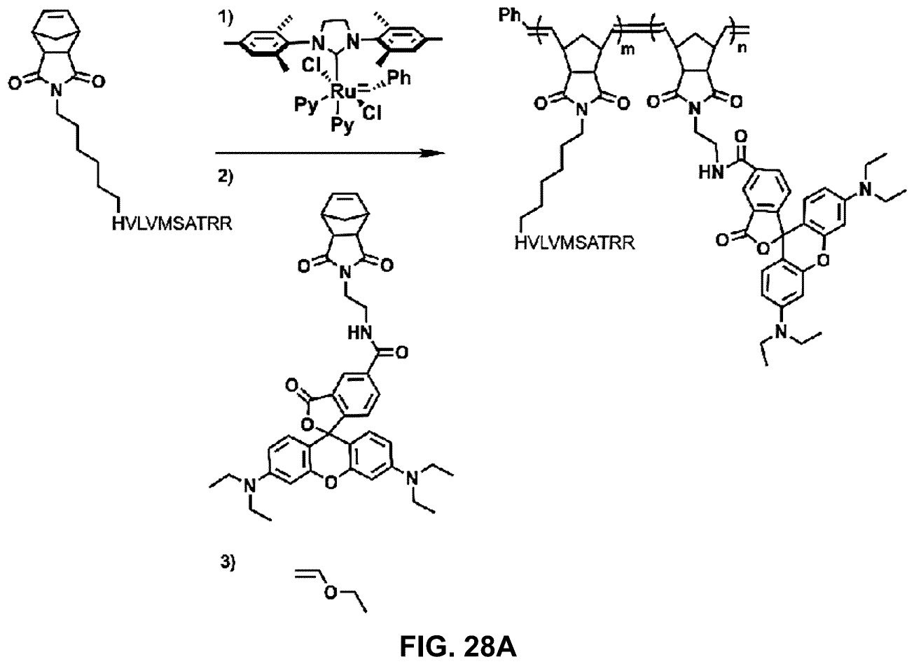

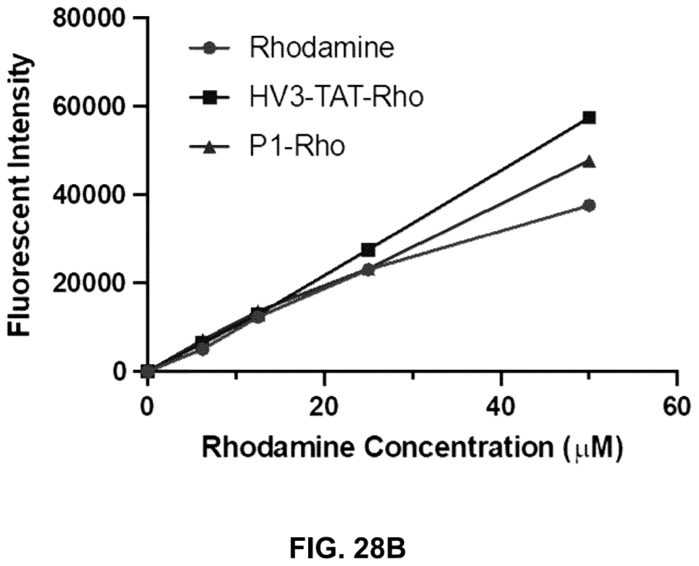

Polymerization: PLPs are achieved by ring-opening metathesis polymerization (ROMP) under nitrogen gas in a glove box (see e.g., FIG. 1A for representative schematic of polymerization). Norbornene conjugated peptide monomers (20 mg, 15.0 equiv., 30 mM) are dissolved in degassed DMF with 1M LiCl. Next, the olefin metathesis initiator (IMesH2)(C5H5N)2(Cl)2Ru=CHPh stock solution (1.0 equiv., 20 mg/mL in DMF) is quickly added into the monomer solution. The solution is left to stir until the full consumption of monomers (e.g., 12 hours). In the case of rhodamine tagged polymers, this is achieved by the addition of 1 eq of a rhodamine linked to norbornene via a six-carbon chain linker with an amide bond (see e.g., FIG. 28A for representative schematic of polymerization). After the polymerization, the polymer solution is precipitated in diethyl ether, and is further purified via dialysis into deionized water. Finally, the polymer product is obtained by lyophilization. Aspects of this process are described in further detail in Kammeyer et al., Polymerization of Protecting-Group-Free Peptides via ROMP, Polym. Chem. 2013, 4 (14), 3929-3933 and Nomura et al., Precise Synthesis of Polymers Containing Functional End Groups by Living Ring-Opening Metathesis Polymerization (ROMP): Efficient Tools for Synthesis of Block Graft Copolymers, Polym. 2010, 51(9), 1861-1881, each of which is incorporated by reference herein in its entirety, and more specifically for methodologies of polymerization, to the extent not inconsistent with the description herein.

¹H NMR is used to confirm complete consumption of the peptide monomer and to determine the time period required to reach completion. The percent conversion of monomer to polymer is tracked over time by comparing a decreasing monomer peak by NMR relative to an internal standard. The linear plot of the NMR reveals the pseudo-first-order kinetics. The polymers are terminated with ethyl vinyl ether (10 eq) for 1 h at room temperature, precipitated and washed with cold diethyl ether three times and collected by centrifugation. Polymers molecular weight and polydispersity are determined by SEC MALS (Phenomenex Phenogel 5μ 103 Å, 1K-75K, 300×7.8 mm in series with a Phenomenex Phenogel 5μ 103 Å, 10K-100K, 300×7.8 mm) at 65° C. in 0.05M LiBr in DMF, using a ChromTech Series 1500 pump equipped with a multi-angle light scattering detector (DAWN-HELIOS II, Wyatt Technology) and a refractive index detector (Wyatt Optilab TrEX) normalized to a 30,000 MW polystyrene standard. Molecular weight may also be determined via SDS-PAGE (sodium dodecyl sulfate-polyacrylamide gel electrophoresis). Polymers are run at 2 mg/ml on precast Mini-PROTEAN TGX gels 4-10% and visualized using Instant Blue or Coomassie Blue Stain.

### Example 1

This example describes the synthesis and characterization of certain PLPs.

Peptide Preparation: The HV3 peptide sequence, SEQ ID: 5 (HVLMCAT), was modified to prevent the possibility of disulfide bond formation and aggregation between peptide-sidechains on the polymers, by substitution of cysteine to serine to give SEQ ID NO: 9 (HVLVMSAT). In addition, two or four positively charged arginine or lysine residues were introduced to facilitate cellular uptake (FIG. 1B). The four resulting peptide sequences (peptides of SEQ ID NOs: 1-4 as shown in FIG. 1B) were synthesized and conjugated to N-(hexanoic acid)-exo-5-norbornene-2,3-dicarboximide at the N-terminus using solid phase peptide synthesis resulting in monomers M1-M4.

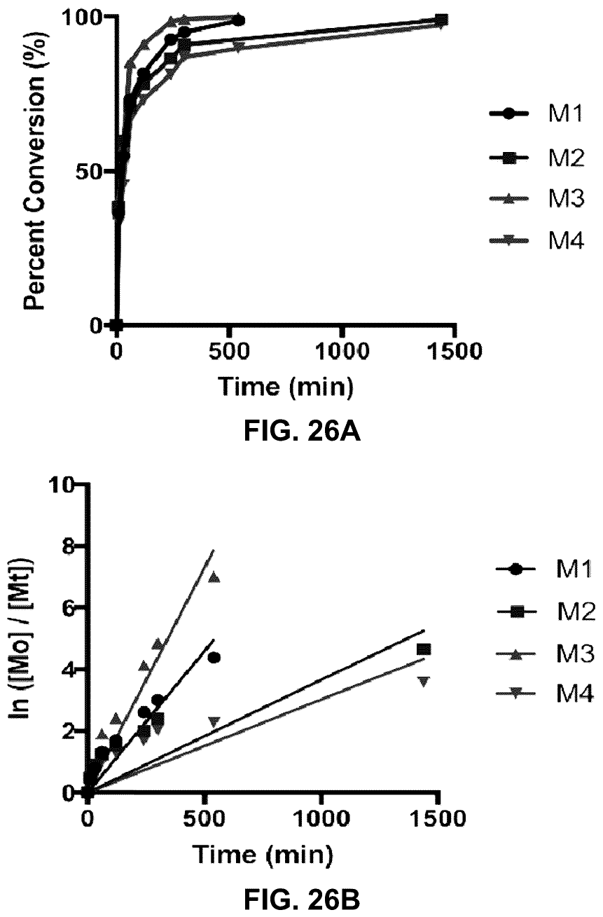

Polymerization: Polymerization reactions were performed on HPLC purified peptide-monomers via ring-opening metathesis polymerization (ROMP) with the Grubbs' ruthenium initiator, (IMesH₂)(C₅H₅N)₂(Cl)₂Ru=CHPh, and were monitored by ¹H NMR (as shown in FIGS. 26A and 26B, where the percent conversion of monomers was determined by integration of olefin peaks in ¹H NMR). For additional information on the Grubbs' ruthenium initiator, see Sanford, M. S.; Love, J. A.; Grubbs, R. H. A Versatile Precursor for the Synthesis of New Ruthenium Olefin Metathesis Catalysts. Organometallics 2001, 20, 5314-5318, which is hereby incorporated by reference in its entirety to the extent not inconsistent with the disclosure herein.

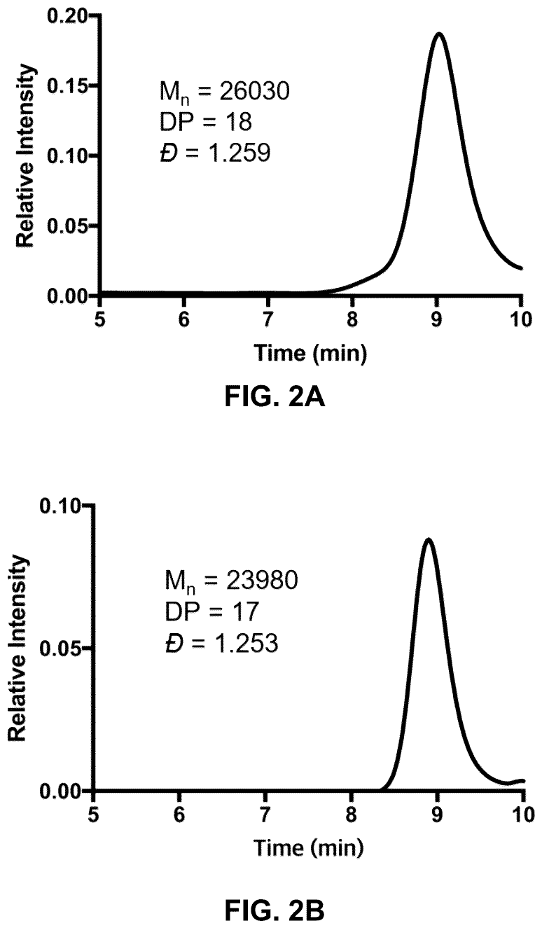

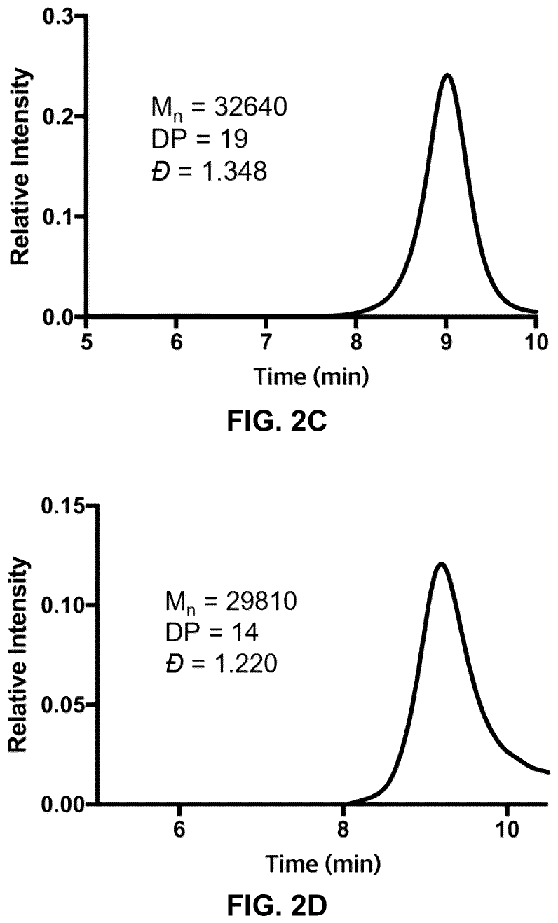

Synthesis and Polymerization Results: The resulting PLPs generated from the four peptide monomers M1-M4, named P1, P2, P3, and P4 respectively, were then characterized via size exclusion chromatography with a multi angle light scattering (SEC-MALS) detector. FIG. 2A depicts the results for P1, where Mₙ was 26,030 Da, DP was 18, and dispersity (D) was 1.259. FIG. 2B depicts the results for P2, where Mₙ was 23,980 Da, DP was 17, and D was 1.253. FIG. 2C depicts the results for P3, where M. was 32,640 Da, DP was 19, and D was 1.348. FIG. 2D depicts the results for P4, where Mₙ was 29,810 Da, DP was 14, and D was 1.220.

### Example 2

This example provides an in vitro assessment of efficacy of the PLPs generated in Example 1 as compared to the efficacy of HV3-TAT.

Cell Viability Methods: In vitro assays were carried out to examine the bioactivity of the PLPs, P1, P2, P3, and P4 and to compare against the known, active HV3-TAT. First, to demonstrate cell rescue, HdhQ111 cells were serum-starved prior to treatment with HV3-TAT and P1 and subsequently assessed for cell viability. HdhQ111 cells were immortalized from knock-in mice carrying 111 CAG repeats in the mouse Htt gene.

VCP Binding Assay Methods: The HV3-TAT peptide and PLPs are designed to selectively inhibit the interaction between VCP and mtHtt as binding of VCP/mtHtt triggers neuronal mitophagy, ultimately leading to cell death. Therefore, target engagement protein binding assays were conducted. HEK293 cells were cultured and plated one day before treatment. Cells were pre-treated with HV3-TAT or PLPs at 3 μM for 2 h. Cells were then co-transfected with GFP-VCP and myc-Q73 FL plasmids. Cells were treated with a second dose of peptide or PLPs 6 hours after transfection, and a final third dose of peptide or PLPs treatment was administered 24 hours after transfection. Cell total lysate was harvested 48 hours after transfection, and to confirm binding of PLPs to VCP in cells, association of VCP to mtHtt was quantified by immunoprecipitation (IP) followed by western blot upon treatment of HV3-TAT peptide and PLPs.

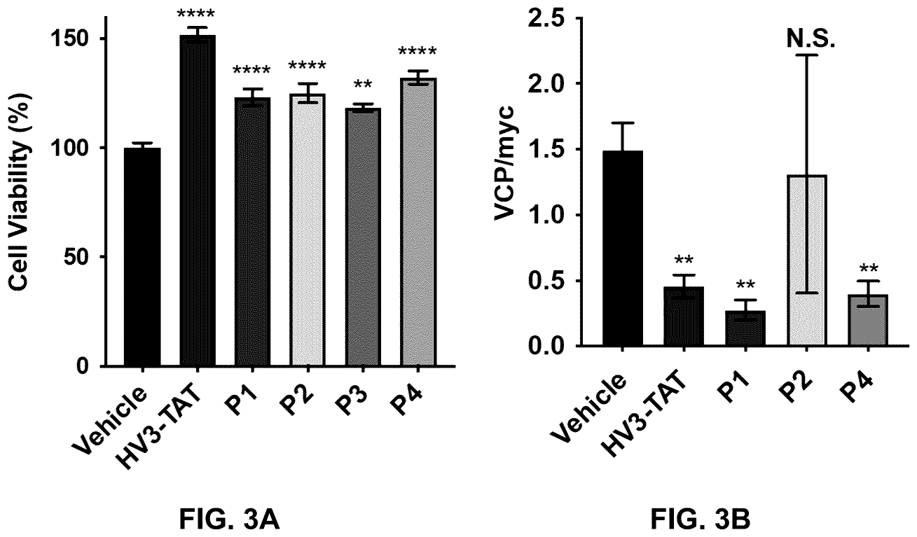

Cell Viability and VCP Binding Results: Compared to the vehicle control, both the HV3-TAT peptide and PLPs showed significant increases in cell viability (FIG. 3A). From these studies, P1, P2, and P4 were selected for the VCP association assay due to higher significance than P3 in the initial cell viability, rescue screen. With respect to the association assay, HV3-TAT peptide, P1 and P4 exhibited evidence for binding (FIG. 3B). Therefore, P1 and P4 were selected to move forward.

### Example 3

Based on the results acquired in Example 2, this Example 3 continued to assess the efficacy of PLPs as compared to HV3-TAT.

Cell Uptake Methods: A major drawback for most peptide-based drugs is poor innate cell membrane permeability, such that many peptide-based drugs fail to reach their putative intracellular targets. Cellular uptake was evaluated by confocal microscopy and flow cytometry using a rhodamine dye labeled version of HV3-TAT (HV3-TAT-Rho) and a rhodamine labeled version of P1 (P1-Rho, e.g., as shown in FIG. 28A). FIG. 28B provides the relative fluorescent intensities of rhodamine alone, HV3-TAT-Rho, and P1-Rho—rhodamine showing near 1:1 addition of dye on to compounds.

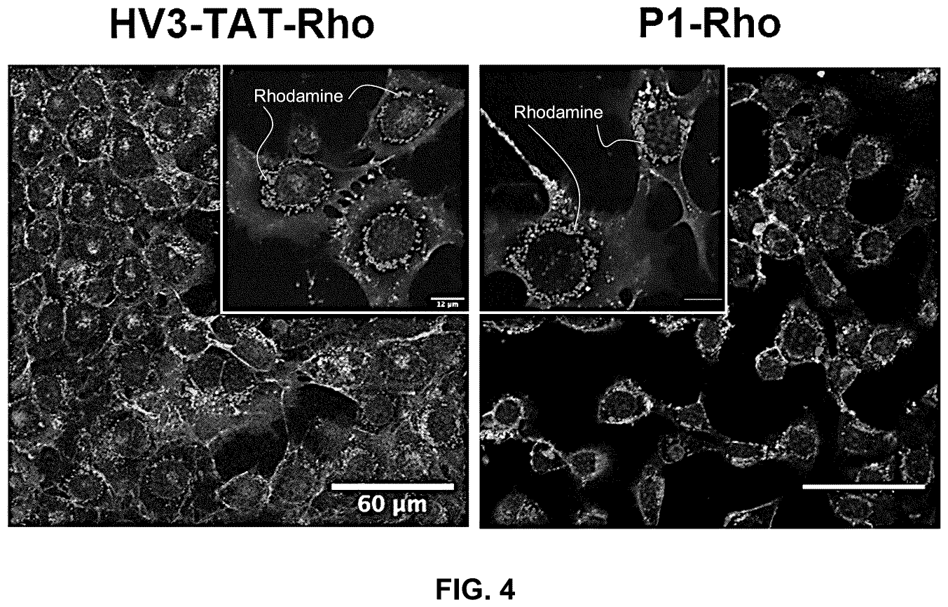

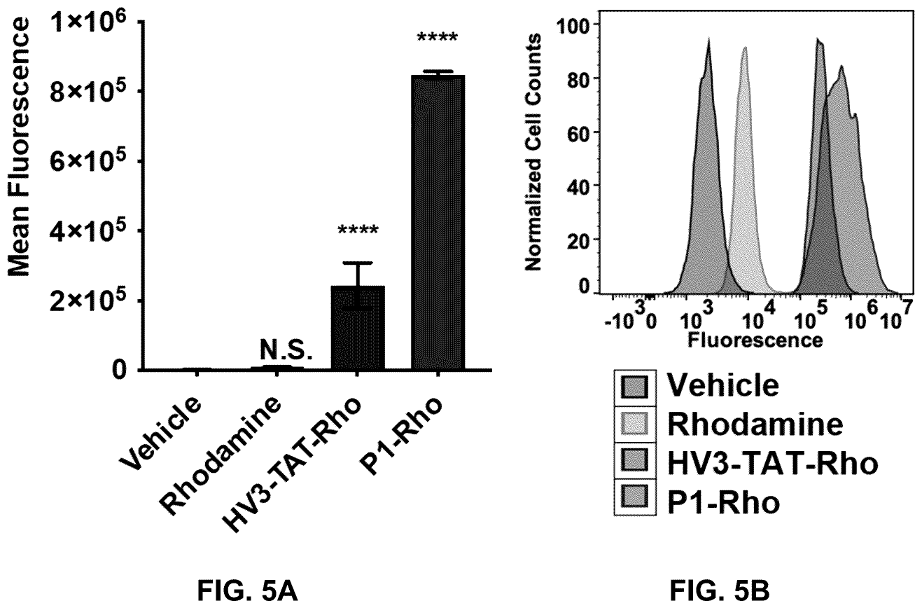

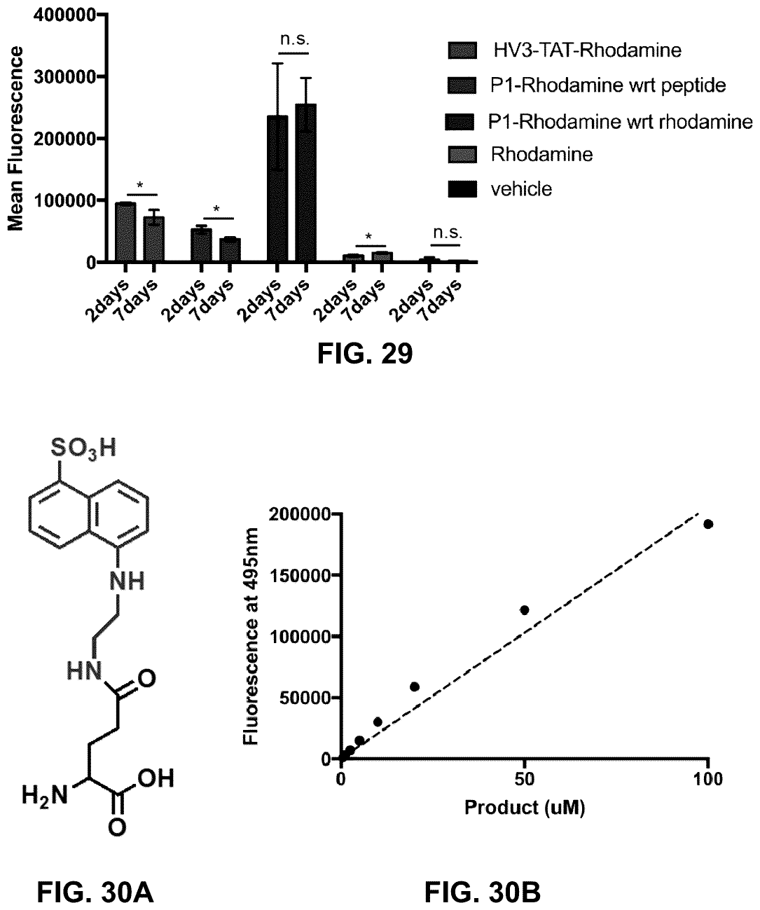

Cell Uptake Results: As shown in FIG. 4, distinct intracellular signals were observed for both dye-labeled treatment groups, confirming the ability of the PLPs to penetrate the cellular membrane. Further, as evaluated by flow cytometry and depicted in FIG. 5A and FIG. 5B, P1-Rho showed significantly better cell association compared to the HV3-TAT-Rho peptide and rhodamine dye control. Further, P1-Rho was detected in the HdhQ111 cells at two and seven days after one initial treatment (FIG. 29).

Mitochondrial Localization Methods: A decisive pathologic hallmark for HdhQ111 cells is mitochondrial fragmentation. Since the mitochondrial membrane is the specific intracellular target location for the designed system, mitochondrial localization of P1-Rho in HdhQ111 cells was evaluated. To visualize mitochondrial localization, the mitochondria were stained with Mito Tracker.

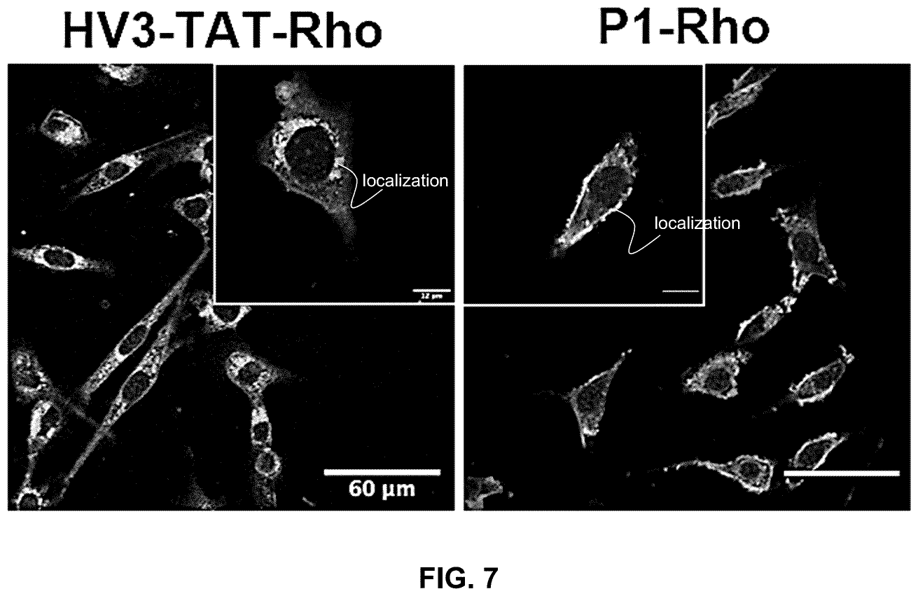

Mitochondrial Localization Results: As shown in FIG. 7, Both HV3-TAT-Rho (positive control) and P1-Rho treated cells exhibited overlapping signals with mitochondria, confirming localization. This suggests that P1-Rho can be delivered intracellularly and subsequently localize to the mitochondrial membrane.

Mitochondrial Fragmentation Methods: As preventing the fragmentation of the mitochondria is important for maintaining the health of striatal cells in the HD in vitro model, the PLP's abilities were tested in this regard. To demonstrate the ability of the PLPs (specifically, P1 and P4) to prevent mitochondrial fragmentation, confocal fluorescent imaging was used to assess mitochondrial morphology after treatment.

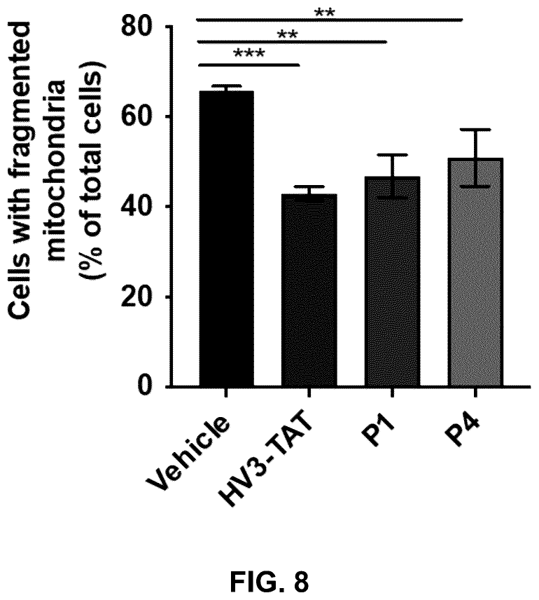

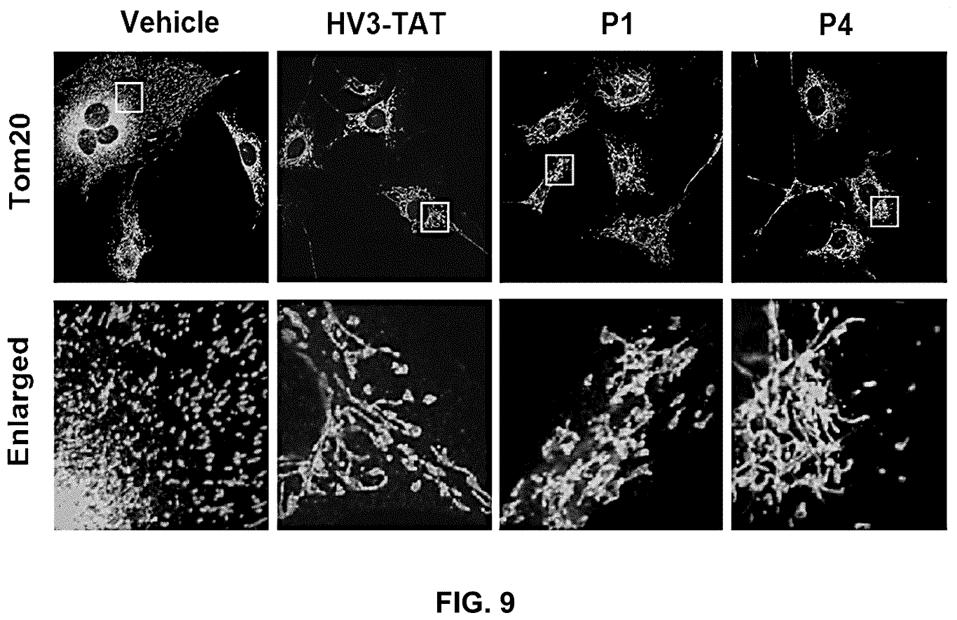

Mitochondrial Fragmentation Results: While mitochondrial fragmentation was observed for the vehicle treatment group, significantly fewer fragmented mitochondria were identified for the HV3-TAT, P1 and P4 treatment groups, as shown in FIG. 9. The percentage of mitochondrial fragmented cells were then quantified with at least 100 cells per group (FIG. 8) and P1 was selected for further analysis.

### Example 4

This Example 4 evaluated the relative binding affinities of VCP with P1 and HV3-TAT.

BLI Methods: With cellular VCP engagement verified as discussed in Example 2 (and shown in FIG. 3B), P1 was further assessed for VCP binding via Bio-layer Interferometry (BLI) to further study the interaction compared to HV3-TAT peptide. Binding affinity was measured by using BLItz instrument. The anti-HIS sensor (HIS2) was hydrated before the installation. After the sensor installation to the BLItz instrument, histidine tagged VCP proteins (50 μg/ml in sodium acetate buffer at pH 6.5) were immobilized on the sensor. HV3-TAT and P1 were dissolved in Tris buffer (pH 8.4) with a range of concentrations. Using BLItz instrument, the dissociation constant (K_{D}), on-rate (kₐ) and off-rate (k_{d}) of each analyte were measured.

Table A. Summary of binding constants of BLI. Binding affinity of HV3-TAT peptide and P1 as measured by bio-layer interferometry. Binding kinetics of HV3-TAT peptide and P1 were measured using VCP protein.

| Constant | HV3-TAT | P1 |
|---|---|---|
| K_{d} (M)^{a} | 1.4 × 10⁻⁵ | 9.2 × 10⁻⁸ |
| kₒₙ (1/Ms) | 1.6 × 10³ | 2.3 × 10³ |
| k_{off} (1/s) | 2.3 × 10⁻² | 2.5 × 10⁻⁵ |

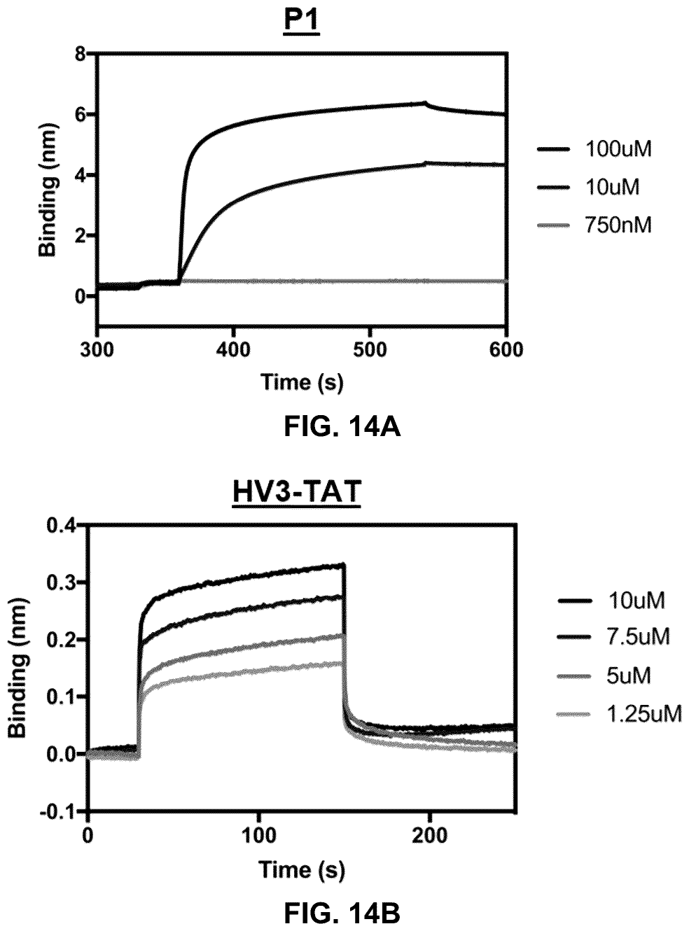

BLI Results: As depicted in FIG. 14B and Table A, a K_{d} of 92 nM was determined for P1, driven by slow k_{off} rates indicative of cooperative binding. This indicates a more than 150 fold decrease in dissociation constant compared to the HV3-TAT peptide at a K_{d} of 14 μM (FIG. 14A). The superior affinity of the PLP compared to free peptide demonstrates multivalency as an inherent attribute of the system Example 5

This example explored P1's ability to permeate the blood brain barrier via in vitro experimentation.

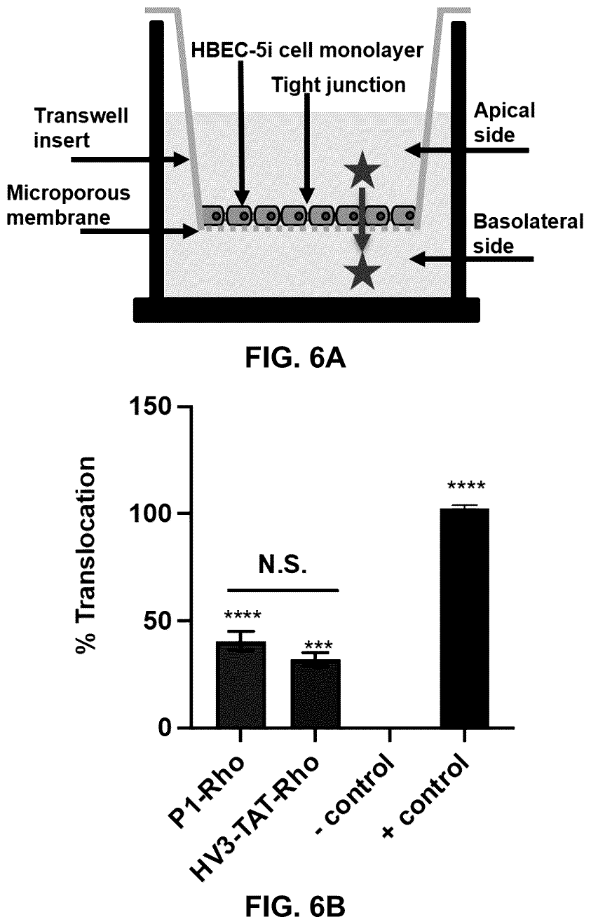

In vitro BBB Methods: To further understand and predict how the PLP would behave in vivo for a neurodegenerative disease target, the ability of P1 to permeate the blood brain barrier was assessed in vitro. Human brain microvascular endothelial cells (HBEC-5i) were used to mimic the highly selective border of endothelial cells in the cerebral vasculature, termed the blood brain barrier (BBB) as depicted in FIG. 6A. Human cerebral microvascular endothelial cells (HBEC-5i, ATCC© CRL-3245) were purchased from American Type Culture Collection (ATCC). HBEC-5i cells were cultured according to manufacturer's instructions: as a monolayer on 0.1% gelatin coated T-flasks in DMEM:F12 medium supplemented with 10% FBS, 1% penicillin/streptomycin antibiotic solution, and 40 μg/mL endothelial growth supplement (ECGS). Cells were grown in an atmosphere of 5% CO₂ at 37° C., with the medium changed every other day, until they reached confluence. Then, cells were detached with trypsin-EDTA and seeded 8,000 cells/well to 0.1% gelatin solution coated tissue culture inserts (transparent polyester (PET) membrane with 1.0 m pores) for 24-well plates (BD Falcon, United States). Over the course of 8 days, the medium was changed every 2 days. After 8 days, cells were washed two times with PBS and once with DMEM:F12 medium without phenol red. Then, P1, HV3-TAT, and controls (Sodium Fluorescein, rhodamine) diluted in DMEM:F12 medium without phenol red to a final concentration of 3 M were added to the apical side of the in vitro BBB model. Translocation was assessed using a plate reader to measure the absorbance of rhodamine, and calculated as % Translocation=(fluorescence outer well—fluorescence from cell media)/(fluorescence control from standard curve of material).

In vitro BBB Results: As depicted in FIG. 6B, compared to the untreated control, both HV3-TAT and P1-Rho showed similar translocation across the in vitro BBB cell layer.

### Example 6

In this example, the proteolytic resistance and maintenance of cellular bioactivity was evaluated.

PLPs have been shown to confer proteolytic resistance on the peptide sidechains driven by polymer backbone hydrophobicity and the resulting globular PLP structure. As discussed, for example, in Sun, H. et al. Origin of Proteolytic Stability of Peptide-Brush Polymers as Globular Proteomimetics. ACS Cent. Sci. 2021, 7 (12) and Gianneschi, N. C. et al. Biomolecular Densely Grafted Brush Polymers: Oligonucleotides, Oligosaccharides and Oligopeptides. Angew. Chemie Int. Ed. 2020, each of which are incorporated by reference in its entirety, and specifically for evidence of proteolytic resistance, to the extent not inconsistent with the description herein. To ascertain the extent to which proteolysis is abated by the peptide sidechains of HV3-based PLPs, enzyme degradation and serum stability studies were conducted.

Stability of PLPs Methods I: For enzyme-induced cleavage experiments, the molar concentration of enzymes was set to 0.1 μM. The concentration of side-chain peptides (having sequence identity of SEQ ID: 1) varied in the range of 50-200 μM. For each enzyme, temperature was set for optimal enzyme activity. In a typical experiment, P1 or HV3-TAT was dissolved in DPBS (pH 7.4) or hydrochloric acid solution (pH 2), to obtain a stock polymer/peptide solution with a peptide concentration of 200 μM. Next, 5 μL of enzyme stock solution (10 μM) was added into 500 μL of the polymer/peptide solution which was subsequently stirred in a preheated oil bath at 37° C. In this case, the molar ratio of peptide substrate to enzymes was 2000:1. During the cleavage, aliquots were taken for HPLC analysis at predetermined time points. Each degradation experiment was repeated three times. Enzyme pretreated P1 was also run on SDS-PAGE and visualized with Coomassie Blue staining.

To verify that the PLPs retain bioactivity after being exposed to enzymes or serum treatments, the viability of HD mouse mutant striatal HdhQ111 cells was assessed following treatment with HV3-TAT and P1. Mouse striatal HdQ111 neurons were grown in Dulbecco's modified Eagle medium (DMEM) supplemented with 10% fetal bovine serum (FBS), 1% penicillin-streptomycin. Cells were maintained at 33° C. and 5% CO₂ with a relative humidity of 95%. HdQ111 cells were plated in 96-well plates at a density of 5000 per well and then left to attach for 24 h. Cells were treated with HV3-TAT or PLPs at 3 μM with respect to peptide once a day, for 3 days. The medium was replaced every day. After 72 h, cells were washed 3 times with PBS buffer. At this point, 20 μL of CTB reagent in 80 uL of medium was added, and the cells incubated for 3 hours at 37° C. Fluorescence was measured at 590 nm with excitation at 560 nm using a Perkin Elmer EnSpire plate reader. The average background fluorescence of CTB in media without cells was subtracted from the average fluorescence readings of the experimental wells. Viability was calculated as the average background-subtracted signal in a well compared to that of a negative control well (treatment with vehicle or media). Three replicates were performed for each independent sample. 10% DMSO was used as a positive control and untreated cells in complete medium as a negative control. Viability is reported as a percentage of untreated cells.

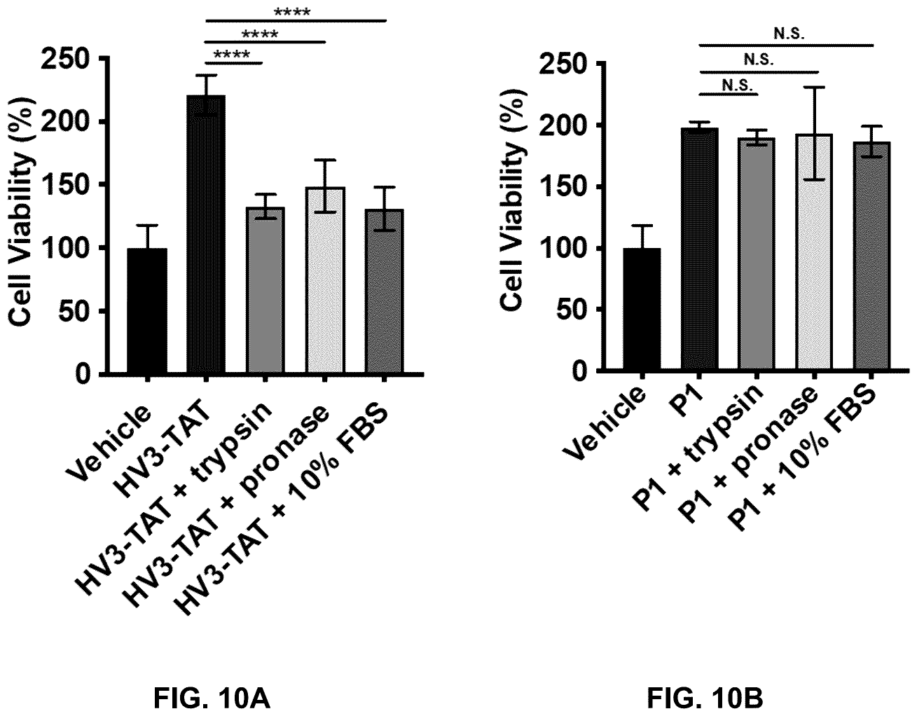

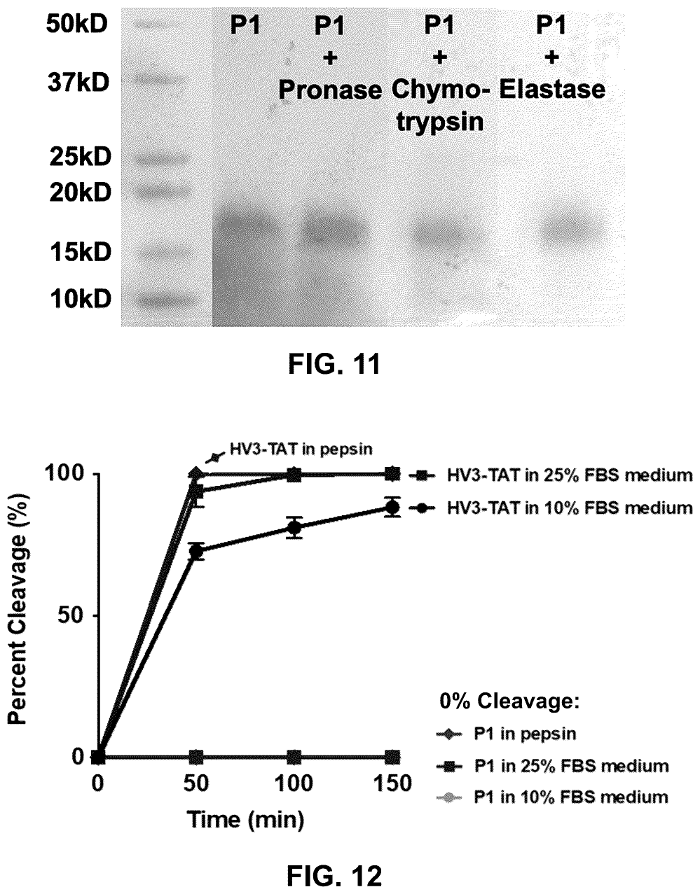

Stability of PLPs Results I: Cells treated with enzyme or serum pretreated HV3-TAT peptide showed a significant decrease in cell viability relative to those treated with undigested peptide (FIG. 10A). Notably, P1 did not have any significant change in cell viability between groups with and without pretreatment with enzyme or serum (FIG. 10B). For SDS-PAGE, enzymatically treated P1 maintained molecular weight running together with the untreated P1 control (FIG. 11), demonstrating proteolytic stability.

Stability of PLPs Methods II: To further confirm the enhanced proteolytic resistance of PLPs compared to the free HV3-TAT peptide, each compound was separately incubated with pepsin and subsequently analyzed using analytical HPLC to evaluate specific peptide fragments and to determine percent cleavage as a function of time. Pepsin is a common gastric enzyme which was selected for its ability to fragment the HV3 peptides at only one cleavage site. As such, fragmentation could be confirmed by comparing the HPLC traces of the enzyme-treated compounds with that of the synthesized peptide fragment generated by pepsin (SEQ ID NO: 10 (SATRR)). Stability of HV3-TAT and P1 were also assessed by incubating materials in serum (10% fetal bovine serum (FBS) rich media, and 25% FBS rich media). Polymers/peptide (200 μM with respect to peptide) were incubated in Dulbecco's modified Eagle medium (DMEM) supplemented with 10% or 25% fetal bovine serum (FBS) at 37° C. At each timepoint, 50 μl of cold acetonitrile with 0.1% trifluoroacetic acid was added to 50 μl of aliquot in order to deproteinize the plasma proteins and subjected to centrifugation at 4° C. for 15 min at 10,000 rpm. The supernatant was injected to the HPLC to analyze degradation kinetics.

Stability of PLPs Results II: As shown in the HPLC results of FIG. 12, the HV3-TAT peptide was 100% cleaved into two fragments in only 50 min, while P1 remained protected, with no cleavage observed for up to 450 min. The HV3-TAT peptide continued to show rapid degradation under both 10% and 25% FBS conditions, in contrast to the stability of P1 seen with serum treatments (FIG. 12).

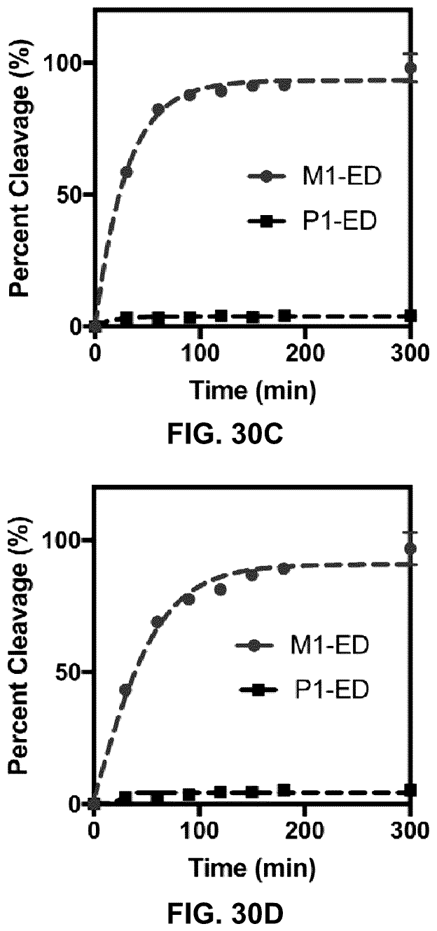

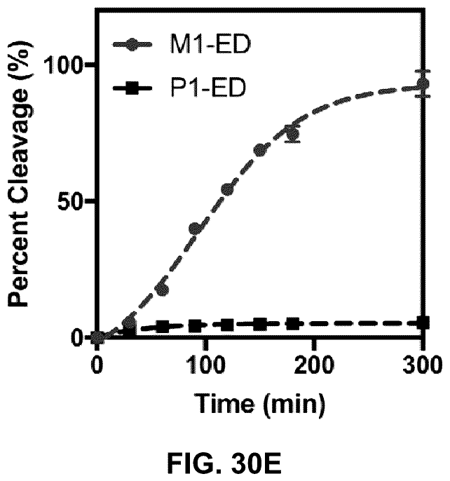

Synthesis and Enzyme Resistance of Fluorogenic Polymer Methods: P1's resistance to enzyme degradation was further tested by preparing a donor-acceptor labeled fluorogenic substrate (EDANS-DABCYL pair). First, EDANS-DABCYL monomer was synthesized. Fmoc-Lys(dabcl)-OH (Sigma) and Fmoc-Glu(EDANS)-OH (Sigma) were coupled on a peptide synthesizer to opposing ends of the peptide sequence of M1. The chemical structure of EDANS-glutamic acid is shown in FIG. 30A for reference. M1-ED monomer was polymerized into EDANS-DABCYL conjugated HV3-PLP (“P1-ED”) via ROMP under nitrogen gas in a glove box. M1-EDANS-DABCYL (8.73 mg, 10.0 equiv.) was dissolved in degassed DMF with 1M LiCl. Next, the olefin metathesis initiator (IMesH2)(C5H5N)2(Cl)2Ru=CHPh stock solution (0.3 mg, 1.0 equiv) was quickly added into the monomer solution. The solution was left to stir for 12 h until the full consumption of monomers. After the polymerization, the polymer solution was precipitated in diethyl ether, and was further purified via dialysis into deionized water. Finally, the polymer product was obtained by lyophilization. Different enzymes (trypsin, thermolysin, and elastase, each at 0.1 μM) were treated to the EDANS-DABCYL conjugated monomer (M1-ED) and polymer (P1-ED). The concentration of M1-ED and P1-ED was set to 50 μM with respect to peptide. The fluorescence changes were observed at 495 nm by plate reader for 300 min and the percent cleavage was calculated based on the calibration curve shown in FIG. 30B. In evaluating the polymerization kinetics (not shown) the absence of the monomer olefin proton peak from the δ=6.55 ppm and the corresponding appearance of two broad cis- and trans-olefin proton peaks from δ=5.8-6.0 ppm confirmed the completion of the polymerization reaction.

Synthesis and Enzyme Resistance of Fluorogenic Polymer Results: In these experiments, fluorogenic P1-ED demonstrated resistance to enzyme degradation with little observed change in fluorescence intensity, compared to clear dequenching of EDANS for the fluorogenic monomer peptide when treated with the three enzymes, trypsin (FIG. 30C), thermolysin (FIG. 30D) and elastase (FIG. 30E).

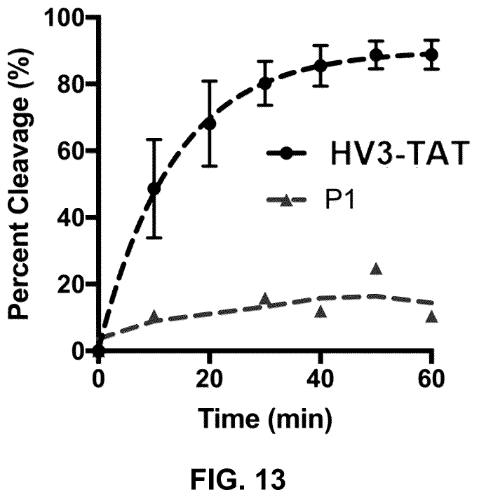

Liver Microsomal Stability Methods: Finally, a liver microsome assay was conducted using P1 and HV3-TAT. P1 or HV3-TAT (2 mg/ml) were incubated in pooled human liver microsome (0.5 mg/ml) activated by 1 mM of NADPH co-factor. At each time point, 10 μL of supernatant was diluted with 90 μL of water with 0.1% trifluoroacetic acid. Degradation kinetics of P1 or HV3-TAT peptide were analyzed by analytic HPLC (FIG. 13).

Liver Microsomal Stability Results: Results from the analytic HPLC in FIG. 13 confirm the superior stability of P1 over HV3-TAT.

### Example 7

In this example, an in vitro analysis of immunocompatibility and hemocompatibility of PLPs was explored.

Immunocompatibility Methods: The interaction of a compound with blood will affect immune response, biodistribution, and clearance. The complement system is a key player in the innate immune response, and thus assaying the interaction between the complement system and PLPs serves as a litmus test to assess inherent inflammatory activity. Complement component C3a is implicated in all three of the complement activation pathways (classic, alternative, and lectin), thus a significant increase in C3a would warrant further investigation into specific activation of the innate and adaptive immune systems. High doses of P1 and HV3-TAT (each at 10 mg/kg), an exemplary therapeutic dose of P1 and HV3-TAT (each at 3 mg/kg), and the PLP backbone were assayed in duplicate using a C3a ELISA assay. In this assay, mouse weight was estimated as 35 g with an average blood volume of 2 mL for dosing preparation. Tested compounds were incubated at a ratio of 1:250 and 1:500 of compound to human serum for 4 hours prior to dilution and start of the assay. Using the C3a ELISA assay, and following manufacturer's protocol, samples were diluted with sample diluent and C3a control reconstituted with water. Cobra venom factor, structurally similar to C3a, was used as positive control, with DPBS as negative vehicle control. Serum samples and controls were incubated in C3a antibody coated wells for 2 hours with gentle shaking, before they were gently washed 3× with washing buffer, and primary antibody biotin solution administered to wells. After an hour of shaking, plate was washed 3× and streptavidin solution added for 1 hour. After a final wash, developing substrate was added for 30 minutes before imaging on plate reader at 450 nM. Results were analyzed via one-way ANOVA performed with respect to mean of P1 at 1.2 μM.

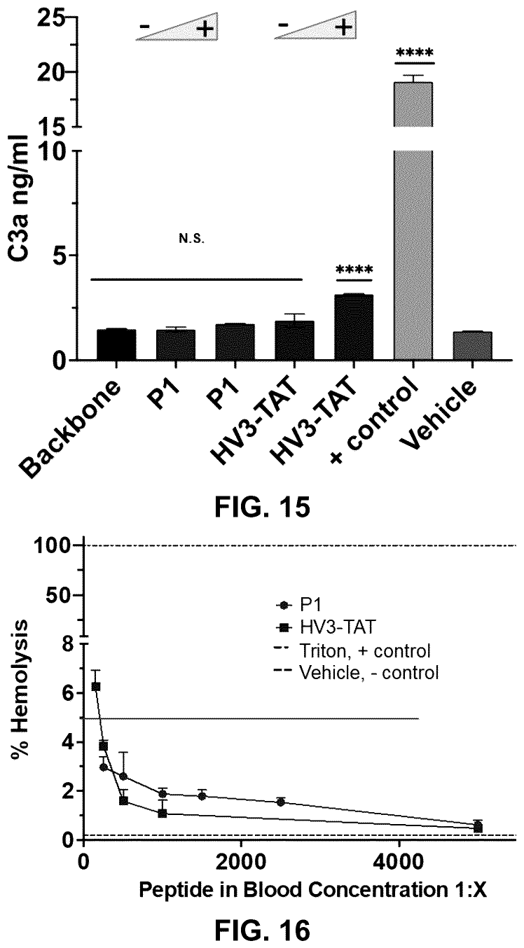

Immunocompatibility Results: In FIG. 15, the first “P1” (leftmost P1) corresponds to the therapeutic dose and the second “P1l” (rightmost P1) corresponds to the high dose, as represented by the icons at the top of the graph. Similarly, in FIG. 15, the first “HV3-TAT” (leftmost HV3-TAT) corresponds to the therapeutic dose and the second “HV3-TAT” (rightmost HV3-TAT) corresponds to the high dose. The PLP backbone and tested concentrations of P1 did not show a statistically significant difference in activity compared to the vehicle, as shown in FIG. 15. HV3-TAT did not show significant activation at the therapeutic concentration of 3 μM but above 60 μM, significant activation was observed.

Hemocompatibility Methods: Before in vivo studies could be conducted, hemocompatibility of the compounds was also assessed via activated clotting time (ACT) and hemolytic activity. Peptide-in-blood and polymer-in-blood dilutions were performed at volume ratios of 1:10000, 1:5000, 1:1000, 1:500, 1:250, and in some cases 1:100. Given efficient distribution after injection, it was expected that a peptide-in-blood or polymer-in-blood dilution would be between 1:1000 and 1:500, whereas concentration immediately after bolus in tail vein could be as high as 1:250 to 1:100, informing higher concentrations. However, it was anticipated that efficient distribution of compounds (as would be informed by pharmacokinetics) and the injection would take place over the course of minutes, allowing the compounds to diffuse. It should be noted that efficacy would be performed in vivo with compounds released via Alzet pump, resulting in even further blood dilution, and thus our concentration range is sufficient to inform us of hemocompatibility issues even with build up over time at dilute levels.

Regarding the hemolytic activity assay, the hemolytic assessments required 25 ml of whole human blood mixed with Na Citrate anticoagulant to be obtained. To prepare solution of erythrocytes, whole blood was centrifuged at 500 g for 5 minutes, and plasma aspirated. The same volume of plasma removed was replaced with 150 mM NaCl buffer (in Millipore water). Tube was inverted to mix, centrifuged at 500 g for 5 min, and supernatant removed, and this step repeated 2×. Finally, red blood cells (RBC) were resuspended in PBS. 10 μL of compound was added to 190 μL of diluted red blood cell erythrocyte solution in a 96 well plate (n=4). 20% Triton X acted as the positive control, and PBS acted as the negative control/vehicle. Plates were incubated at 37° C. for 1 hour. Plates were centrifuged for 5 min at 500×g to pellet intact erythrocytes. Using a multichannel pipet, 100 μl of supernatant was transferred from each well into a clear, flat-bottomed 96-well plate. Absorbance of supernatants was measured with a plate reader at 405 nM

For ACT assay, ACT of whole human blood was measured using a Hemochron 801 instrument calibrated with an electronic system verification (ESV). To minimize variability in starting time points for clotting in all assays, ACTs were measured using sodium citrate treated whole human blood with calcium chloride added at a specific time. Controls with Type I collagen (0.095 mg/mL, 1:240 dilution) or without calcium both utilized 1×DPBS as a vehicle to ensure consistent blood dilution in all experiments. 4 μL CaCl₂) (1.1M) and 36 μL peptide stock solution (12.2× final blood concentration) was added to each Hemochron P214 tube with glass beads. Samples were mixed, and citrated whole human blood (400 μL) was then added which marked t=0 sec, mixed by hand for 10 sec, and added to the instrument. Clot formation was recorded by the instrument as the time points at which the magnet was displaced by the formed clot. Each experiment was performed n=4 times with averages and standard error plotted. For samples without calcium, blood clotting times exceeded the instrument maximum range (>1500 sec).

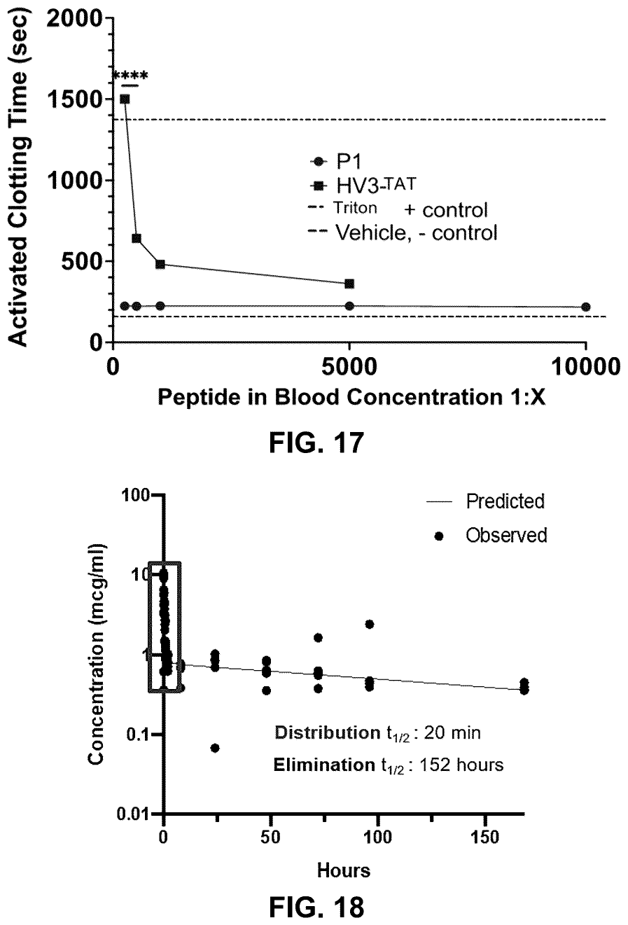

Hemocompatibility Results: In the ACT Hemochron assay, P1 did not show any significant change in ACT compared to the vehicle control (FIG. 16), suggesting that P1 will not induce significant clotting changes. Hemolytic activity of P1 was also found to be non-significant compared to vehicle control (FIG. 17), suggesting that P1 will not induce the breakage of red blood cells in the blood. The results of these in vitro assays suggest that P1, is hemocompatible and non-immunogenic.

### Example 8

Based on the promising results discussed in Example 7, in vivo pharmacokinetic profiling experiments were conducted as detailed in this example.

Synthesis of DOTA Terminating Agent (DOTA-TA) Methods: A gadolinium (Gd) metallated termination agent was prepared to provide a tag for PLPs for in vivo PK analysis using inductively coupled plasma mass spectrometry (ICPMS) allowing quantification in blood and tissue samples. To prepare this tag, first DOTA-TA was synthesized. 51.69 mg (0.10375 mmol) 4,4′-[(2Z)-2-Butene-1,4-diylbis(oxy)]bis[benzeneethanamine] was prepared and deprotected with 3 ml of TFA in 3 ml of DCM for 2 hours and rotovapped to leave a brown oil, and then mixed with DOTA-NHS Ester (Macrocyclics, B-280), 158 mg (0.2075 mmol) in DIPEA (5 mL) and DCM (5 mL) and MeOH (1 mL). The reaction mixture was stirred at room temperature for 48 hours. Solvent was removed in vacuo leaving a brown oil. NMR peaks (not shown) for DOTA-TA: ¹H NMR-D₂O-7.25-6.85 (m, 8H), 5.9 (m, 2H), 4.7 (m, 4H), 4.1-2.6 (m, 32H), 2 (s, 1H), 1.9 (s, 1H). In evaluating the polymerization kinetics (not shown) the absence of the monomer olefin proton peak from the δ=6.55 ppm and the corresponding appearance of two broad cis- and trans-olefin proton peaks from δ=5.8-6.0 ppm confirmed the completion of the polymerization reaction. Additionally, the reduction of the catalyst-monomer olefin proton peak from the δ=18.75 ppm to δ=17.75 ppm and the corresponding appearance of catalyst-terminating agent olefin proton peak at δ=19 ppm confirmed the termination of the polymerization reaction.

Synthesis of Gd-DOTA Terminating Agent Methods: DOTA-TA (35.92 mg) was dissolved in DCM (5 mL), and 2 eq. Gd(OAc)₃ added (25.87 mg, 0.0656 mmol). Reaction stirred for 48 hours at room temperature. Solvent was removed in vacuo to yield Gd-DOTA-TA as a white microcrystalline solid. This powder was purified using preparative HPLC on a gradient of 25%-35% Buffer B (MeCN with 0.1% TFA) in Buffer A (Water with 0.1% TFA). Purified, lyophilized product was characterized via ESI and RP-HPLC. Gd metalation efficiency was confirmed via ICPM-MS. Briefly, 0.68 mg of Gd-DOTA-TA was weighed and digested overnighted, diluted and then run on ICP-MS where metalation efficiency was determined to be 98.99% (not shown).

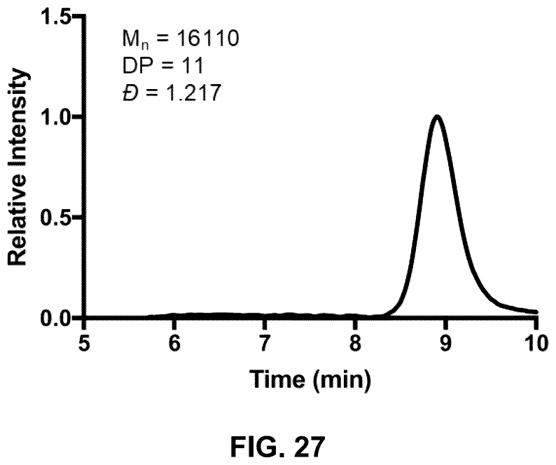

Polymerization of Gd-DOTA-TA and P1 (“P1-Gd”) Methods: P1-Gd was achieved by ROMP, where M1 (88.2 mg, 15.0 equiv., 30 mM) was dissolved in degassed DMF with 1M LiCl. Next, the olefin metathesis initiator (IMesH2)(C5H5N)2(Cl)2Ru=CHPh stock solution (3 mg, 1.0 equiv., 10 mM) was quickly added into the monomer solution. The solution was left to stir for 12 h until the full consumption of monomers. Then, Gd-DOTA-TA was added (8.71 mg, 1.5 equiv., 10 mM). After the polymerization, the polymer solution was precipitated in diethyl ether, and was further purified via dialysis into deionized water. Finally, the polymer product was obtained by lyophilization. Gd metalation efficiency was confirmed via ICPMS. Briefly, serial dilutions of P1-Gd were prepared in Millipore H₂O, digested according to ICP-MS protocol, and ICPMS run, confirming metalation efficiency at 95% (not shown). The resulting characteristics of the P1-Gd are provided in the SEC-MALS results of FIG. 27: M.=16,110 Da; DP=11; and Ð=1.217.

In vivo Pharmacokinetic and Biodistribution via ICPMS Methods: CD1 mice were maintained with a 12-h light/dark cycle (on 06:00 hours, off 18:00 hours) and acclimated for 1 week. Mice were dosed via IV with the P1-Gd at 10 mg/kg with respect to peptide (xx mg/kg of polymer) with a dose for a 30 g mouse being 0.189 mg of polymer in 100 μL of saline. At timepoints of 5 minutes, 10 min, 20 min, 30 min, 40 min, 50 min, 1 hr, 2 hr, 4 hr, 8 hr, 24 hr, 48 hr, 72 hr, 96 hr, and 168 hr, mice were anesthetized and exsanguinated. N=5. Whole blood was collected in pre-weighed falcon tubes. Livers, kidneys, striatum, cortex, and rest of the brain tissue were collected in pre-weighed tubes. Tubes were weighed again, to determine total mass of tissue collected in each tube. To each tube of all samples except liver, 300 μl of conc. nitric acid and 30% hydrogen peroxide were added. Liver samples had 500 μl of conc. nitric acid and 30% hydrogen peroxide added. All samples were digested in a 65° C. water bath for 24 hours, until solutions were transparent. Tubes were diluted to 5 ml total volume (10 ml for liver) with Millipore water, and weighed for a final time. After this point, any excess digested tissue that precipitated was removed by centrifugation and saving the supernatant. Each sample was run on high resolution ICPMS, the Thermo iCAP Q Inductively Coupled Plasma Mass Spectrometer, which, after calibration with known Gd concentrations, resulted in exact Gd concentration in ppb of each sample. Ppb of Gd directly related to the calibration curve of known Gd PLP concentrations, and was used to calculate the amount of PLP present in each sample.

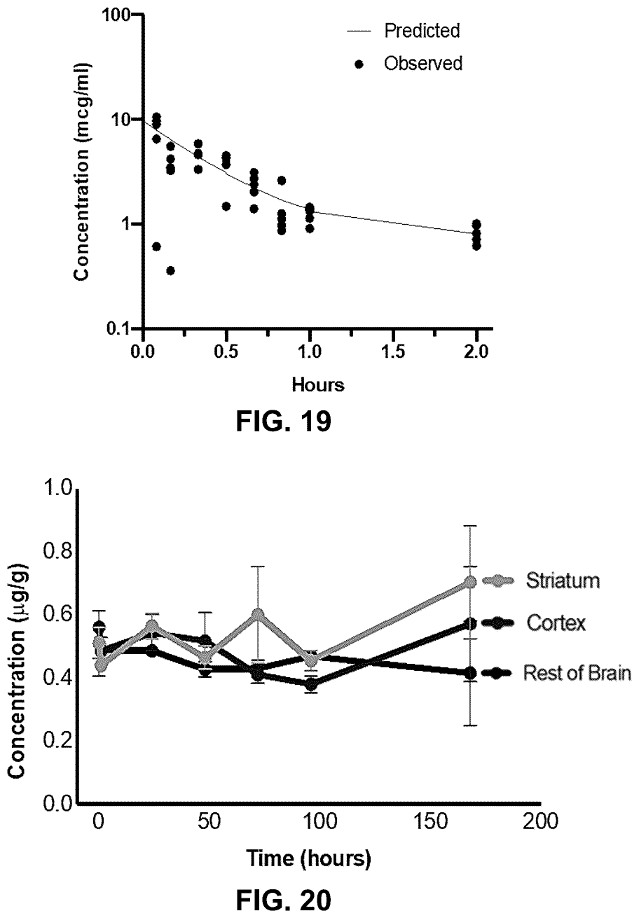

ICPMS Results: The pharmacokinetics of the PLP followed a two-compartment model. Here, an initial 20 min distribution half-life was observed (FIG. 19), followed by an extended 152 hr elimination half-life (FIG. 18). This represents a 3000-fold-increase in half-life compared to the HV3-TAT peptide. This was expected since therapeutic peptides in general are typically hindered by poor pharmacokinetics and half-lives. While the majority of the compound injected ends up in the liver and kidney (not shown), primary sites for drug metabolism, P1-Gd was detectable in the CNS (FIG. 20). Two-way ANOVA showed no significant difference between the concentrations in the brain, striatum, and cortex for all time points (FIG. 20), suggesting an average concentration of 0.6 μg/g is maintained after 5 min, indicating repeated dosing may be needed to reach the target efficacious dose in the brain.

### Example 9

This example discusses experiments conducted to evaluate the toxicity of P1 in WT mice.

Methods: To assess the toxicity of P1 at the administrable dose in healthy animals, as compared to HV3-TAT at its operating dose, and saline for the negative control, male C57BL/6J mice were assigned to 3 groups (n=3-4 per group) and administered via osmotic subcutaneous pump (Alzet) either HV3-TAT at 3 mg/kg, P1 at 3 mg/kg, all with respect to peptide concentration, or saline as a negative control for 8 weeks. Animals were checked weekly after drug administration for mortality. At sacrifice, blood was collected from each animal via cardiac puncture using a 25-gauge needle while under isoflurane, and then animals were sacrificed and major organs (brain, lung, heart, liver, spleen, spinal cord, and kidneys) collected, weighed, and stored in 10% buffered formalin followed by paraffin embedment.

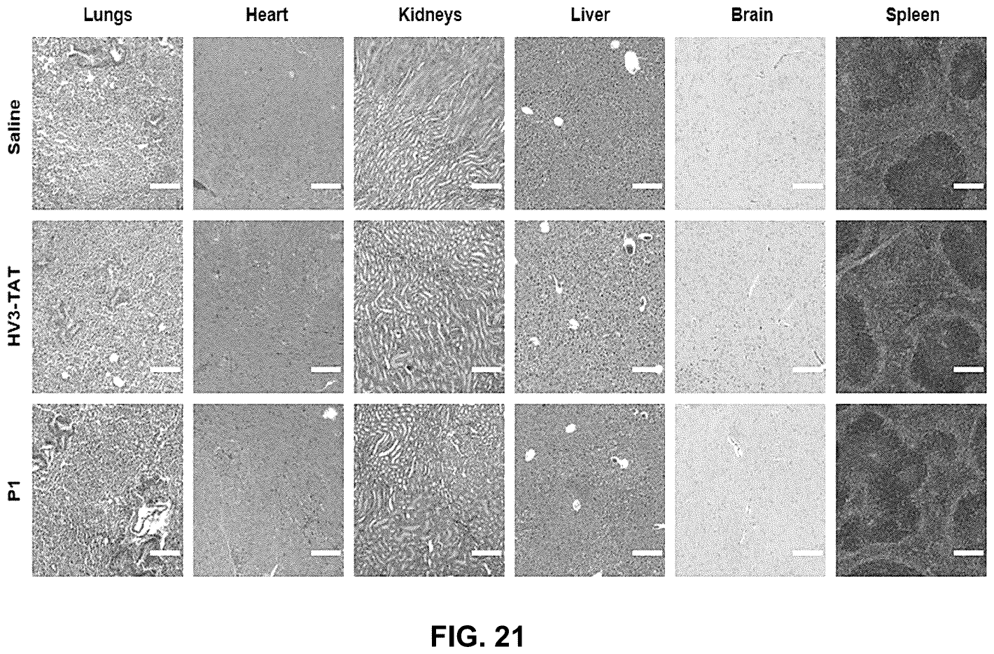

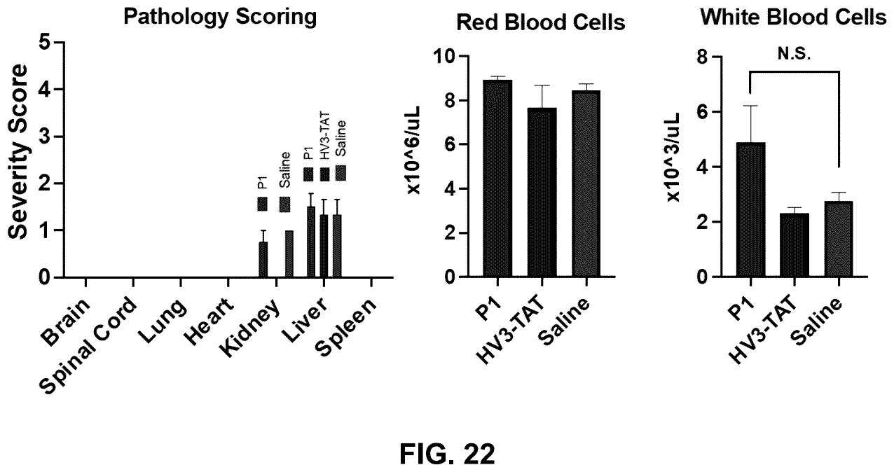

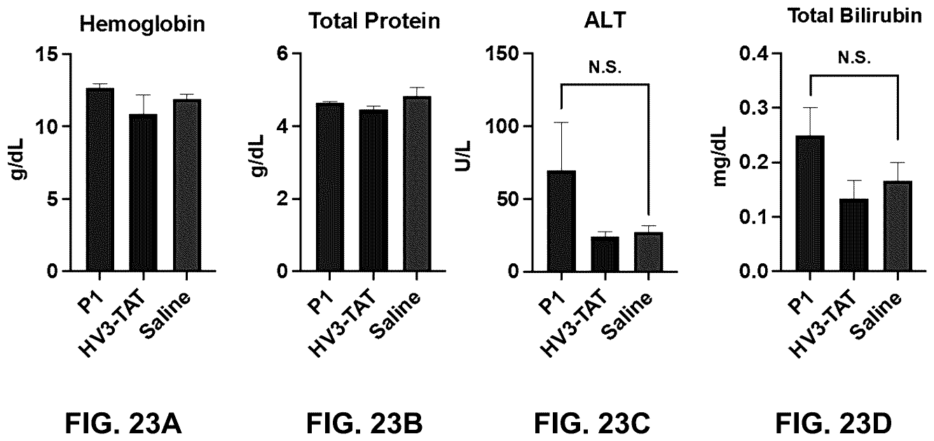

Results: A complete blood count (CBC) panel and serum chemical analysis were obtained with no significant difference between groups (FIG. 22 (middle), FIG. 22 (right), and FIGS. 23A-23D). All values were not significantly different from saline control values, suggesting no toxicity. The tissues were stained with hematoxylin and eosin (H&E), as shown in FIG. 21, and scored by a senior pathologist at Charles River Laboratory (FIG. 22 (left)). No pathological differences between the treated (P1 and HV3-TAT) and untreated (saline) were observed.

### Example 10

In this example, in vivo Huntington's disease model efficacy of P1 is evaluated.

In vivo Efficacy Methods—Behavioral Analysis: All experiments in animals were conducted in accordance with protocols approved by the Institutional Animal Care and Use Committee of Northwestern University and were performed based on the National Institutes of Health Guide for the Care and Use of Laboratory Animals. HV3-TAT at its operating dose, saline (negative control), and P1 at a dose of 1.69 mg/kg/day (3 mg/kg/day with respect to peptide) were administered to Huntington's phenotype R6/2 (sometimes referred to herein as R62) mice and WT mice via Alzet osmotic pump from 6 to 13 weeks of age. The accepted HD phenotype is a decrease in motor coordination, general motility and body weight in male mice as they age from 5 to 13 weeks. A key phenotype of R6/2 mice is hindlimb clasping. Accordingly, hindlimb clasping was assessed with the tail suspension test weekly from the ages 7 to 13 weeks in R6/2 mice and WT littermates. Mice were suspended by the tail for 60 sec and the latency for the hindlimbs or all four paws to clasp was recorded using the following score system: clasping over 10 sec, score 3; 5-10 sec, score 2; 0-5 sec, score 1; 0 sec, score 0. The body weight and survival rate of HD mice and WT littermates were also recorded throughout the study period. For open field motor activity, gross locomotor activity was assessed in R6/2 mice and age-matched WT littermates at the ages of 13 weeks. In an open-field activity chamber (Omnitech Electronics Inc.), mice were placed in the center of the camber and allowed to explore while being tracked by an automated beam system (Vertax, Omnitech Electronics Inc.). The analyzer recorded the time and position of the mice based on beam break information and rapidly analyzed it. The computer software then calculated multiple activity measures, including: total distance traveled; horizontal activities (ambulation-horizontally directed movement) and vertical activities (rearing-vertically directed movement). One-hour locomotor activity analysis was conducted for R6/2 mice and WT littermates.

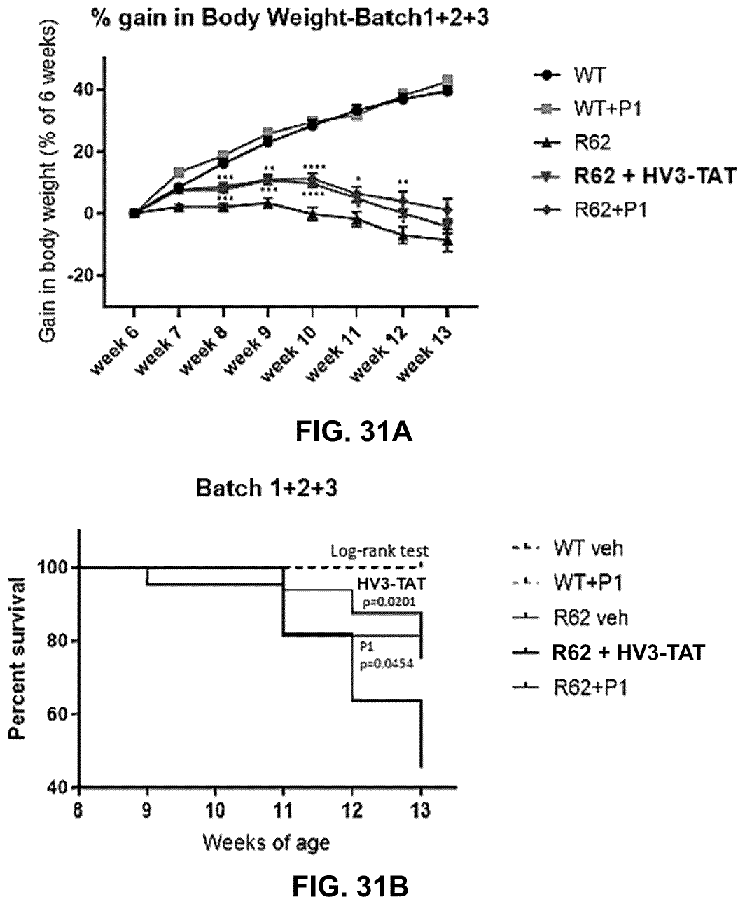

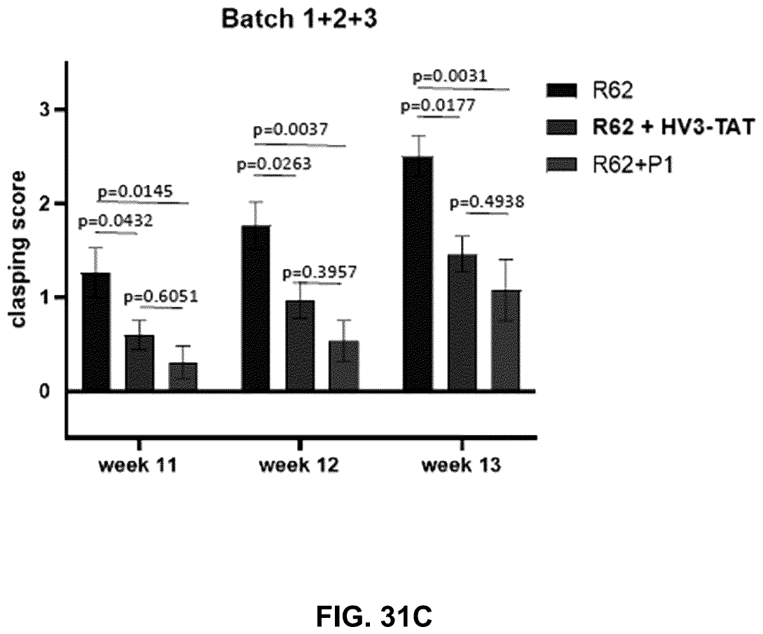

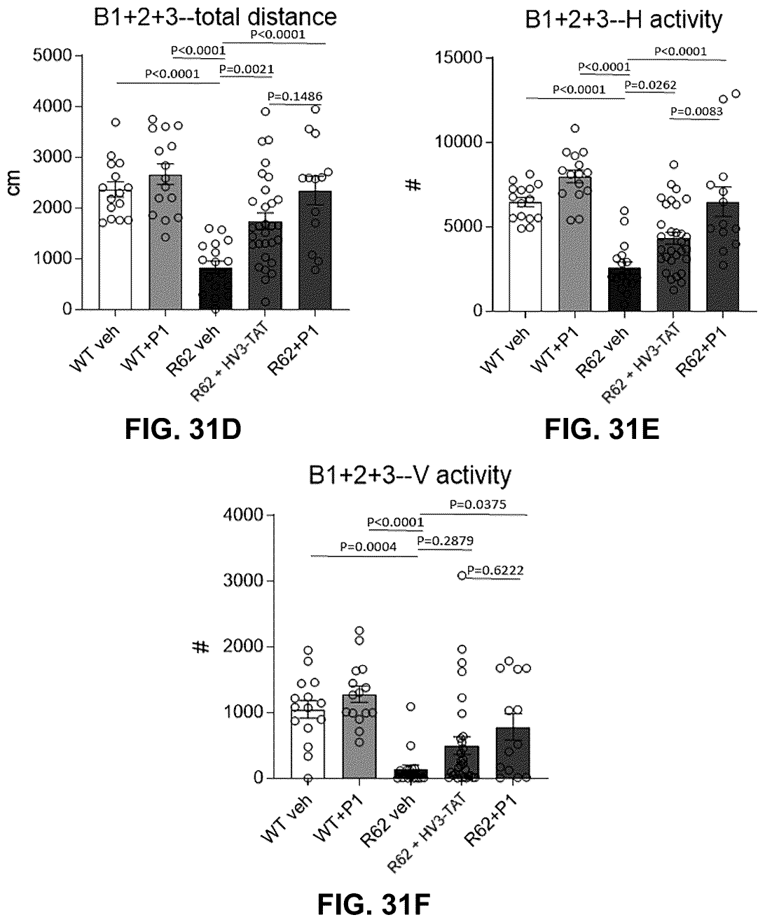

In vivo Efficacy Results—Behavioral Analysis: Disease groups (i.e., R6/2 mice groups) showed a decrease in weight over the course of the study compared to WT controls (FIG. 31A), with P1 and HV3-TAT significantly extending survival over the saline control (FIG. 31B). Hindlimb clasping was assessed weekly, with HV3-TAT peptide treated R6/2 mice exhibiting progressively severe deficits marked by longer clasping time than P1 treated mice (FIG. 31C). Open field motor activity was assessed one week before sacrifice, and P1 and HV3-TAT improved total distance (FIG. 31D), horizontal activity (FIG. 31E), and vertical activity (FIG. 31F). P1 treatment had no significant effects on motor ability, body weight, or neuropathology of WT mice.

In vivo Efficacy Methods—Levels of Proteins of Interest: To further assess the efficacy of P1 in modulating HD neuropathology, the levels of cAMP-regulated phosphoprotein 32 kDa (DARPP-32), post-synaptic density protein (PSD95), and brain derived neurotrophic factor (BDNF) were determined in medium spiny neurons, each of which are decreased in the striatum of HD patients and R6/2 mouse models. Striatal lysates were generated from frozen striatal tissue from un-perfused sacrificed mice. Using a plate reader, protein concentrations were determined by protein assay dye (Bio-Rad). Protein stocks in lysis buffer were mixed with Laemmli buffer, loaded on SDS-PAGE, and transferred onto nitrocellulose membranes (Invitrogen). Membranes were probed with the primary antibodies for actin, DARPP-32, PSD95, and BDNF, and respective IgG-HRP secondary antibodies, followed by visualization of HRP with a blot scanner.

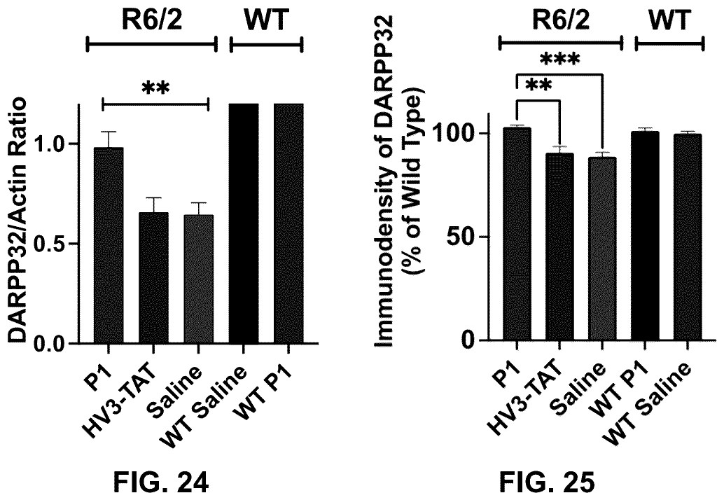

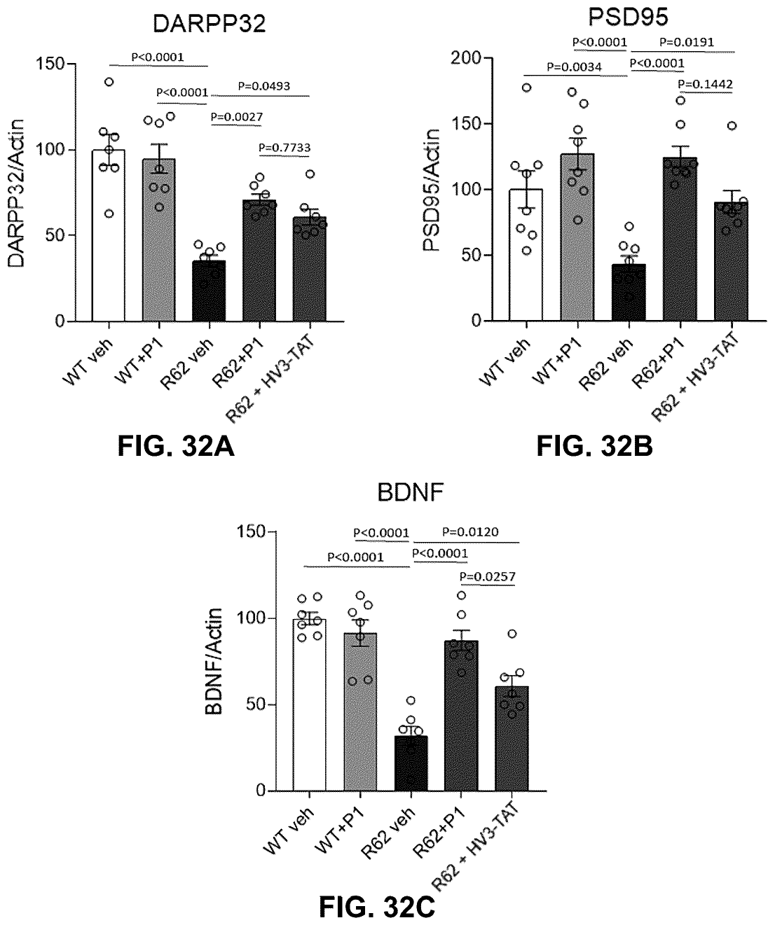

In vivo Efficacy Results—Levels of Proteins of Interest: Western blot analysis of striatal extracts revealed a significant reduction of DARPP-32 protein levels (FIG. 32A), PSD95 protein levels (FIG. 32B), and BDNF (FIG. 32C) in untreated R6/2 mice. HD R6/2 mice treated with P1 had significantly increased DARPP-32 levels over saline control when examined by immunoblotting (FIG. 32A; see also FIG. 24 depicting results from an earlier study). Significant increases of PSD95 and BDNF protein levels were also observed in the immunoblot results (FIG. 32B and FIG. 32C, respectively) for HD R6/2 mice treated with P1 as compared to the saline control. These data sets show that higher levels of each protein of interest is present in brains of PLP treated animals versus control groups, indicating striatal neuron rescue.

In vivo Efficacy Methods—Immunohistochemistry: Mice were anaesthetized with isoflurane and transcardially perfused with PBS. After post-fixed with 4% paraformaldehyde, brains were sliced and frozen. Brain sections (20 μm, coronal) were used for immunohistochemical localization of DARPP-32 (1:14000, Abcam) using the IHC Select HRP/DAB kit (Millipore). Imaging of slices was conducted on an all-in-one fluorescence microscope (Keyence). The same image exposure times and threshold settings were used for sections from all treatment groups. Quantitation of DARPP-32 immunostaining was conducted using NIH image J software.

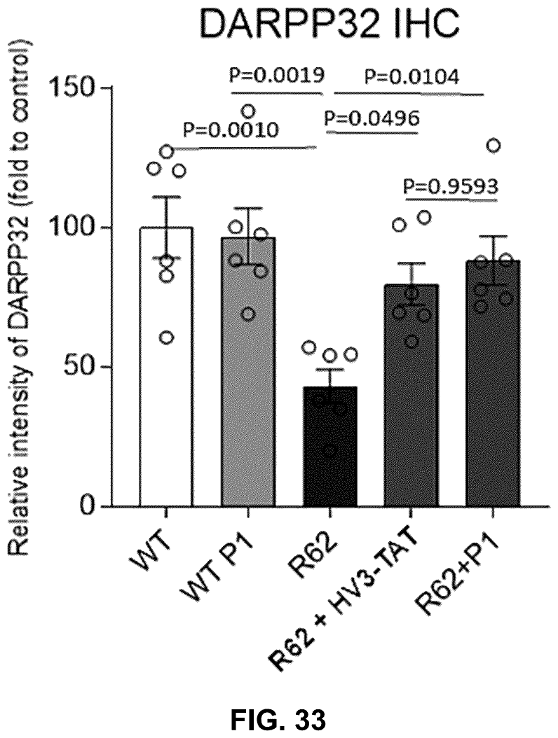

In vivo Efficacy Results—Immunohistochemistry: HD R6/2 mice treated with P1 had significantly increased DARPP-32 levels over saline control when examined by immunohistochemistry (FIG. 33, see also FIG. 25 depicting results from an earlier study), showing similar efficacy to the HV3-TAT peptide. Furthermore, the quantification of histologically stained brain tissue in the striatum showed that P1 was significantly more dense than the saline control (FIG. 33). These results provide additional evidence indicating significantly higher levels of striatal neuron rescue in P1 treated mice when compared to saline treated groups.

In vivo Efficacy Methods—mtHtt Impact: As the pathophysiological hallmark of HD, the formation of mtHtt aggregates can be observed in striatum of HD mouse models, like R6/2 mice. Previous studies have demonstrated that mtHtt is required for VCP accumulation on mitochondria, which causes excessive mitophagy and subsequence neuropathology in HD. HV3 peptide blocks mtHtt-VCP binding, and inhibits VCP mitochondrial accumulation, which reduce behavioral and neuropathological phenotypes of HD. Mitochondrial level of VCP and mtHtt aggregation were examined to test whether PLP recapitulates the mechanism of HV3 peptide. Mitochondrial fractions of striatum were isolated from mice in each treatment group. Protein levels of VCP on mitochondria were analyzed by western blotting, using ATPB as a loading control for mitochondria. mtHtt aggregation was visualized by immunohistochemical staining by anti-mtHtt antibody.

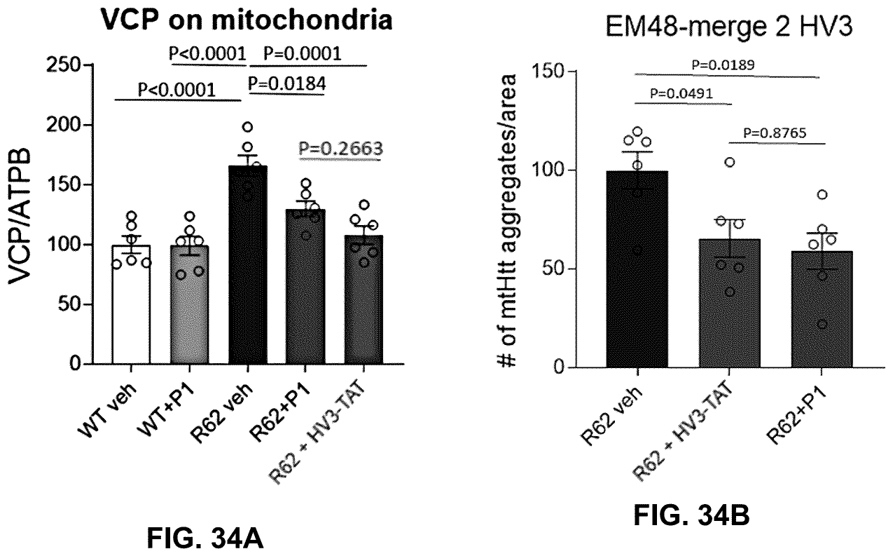

In vivo Efficacy Results—mtHtt Impact: Both HV3-TAT and P1 inhibited mtHtt aggregation showing a reduced number of mtHtt aggregates in striatum compared to saline treated R6/2 mice (FIG. 34B). As shown in FIG. 34A, HV3-TAT blocked aberrant VCP mitochondrial translocation in R6/2 mice showing a comparable level of VCP of mitochondria with WT mice. P1 inhibited mitochondrial VCP accumulation to a lesser extent, but also had a significant effect on it (FIG. 34A).

The combination of results from Examples 1-10 provide support for the treatment of neuropathology with PLPs having therapeutic peptide sidechains capable of VCP/mtHtt inhibition. The PLP exhibited exceptional stability compared to the free peptide, HV3-TAT, yielding increased activity in rescuing neurons following serum incubation of both test articles. PLP significantly extended the distribution half-life (t_{1/2}=20 min) and elimination half-life (t_{1/2}=152 hr) compared to HV3-TAT, with no signs of toxicity despite high and continuous dosing. In vivo efficacy studies for PLP demonstrated similar performance of PLP and HV3-TAT peptide in behavioral studies as well as increases in DARPP-32, PSD95, and BDNF levels, with both treatment groups extending survival compared to saline control groups. Moreover, Examples 1-10 demonstrate the utility of the PLP platform to harness identified bioactive peptide sequences to overcome challenges facing the base peptide therapeutic and to successfully target specific intracellular proteins in the central nervous system.

### Example 11

In this prophetic example, PLP copolymers incorporating peptide side chains with (1) sequences having 75% or greater sequence identity of any one of SEQ ID NOs: 1-5, and (2) additional distinct therapeutic sequences is explored (this PLP copolymer is referred to as “co-PLP” in this Example 11).

SEQ ID NO: 6 (AASSGVSTPGSAGHDIITEQPRS), known in the art as “P42,” is a peptide derived from endogenous Htt. P42 has been shown to target Htt and exhibits anti-mitophagy properties similar to the HV-3 mechanism. Additionally, P42 has been shown to alleviate HD symptoms in R6/2 mice. However, it has not been shown to disrupt preformed Htt aggregates. These characteristics are discussed in detail in Marelli, C.; Maschat, F. The P42 Peptide and Peptide-Based Therapies for Huntington's Disease. Orphanet. J. Rare Dis. 2016, 11 (1), and Couly, S. et al. Improvement of BDNF Signaling by P42 Peptide in Huntington's Disease. Hum. Mol. Genet. 2018, 27 (17), 3012-3028, each of which is incorporated by reference in its entirety, and more specifically to facilitate the understanding of P42, to the extent not inconsistent with the description herein. Given the targeting and therapeutic properties of P42 with respect to HD, it is believed SEQ ID NO: 6, or a sequence having 75% or greater sequence identity of SEQ ID NO: 6, when included as part of the co-PLP may function as both a targeting peptide and/or a therapeutic payload of the co-PLP.

SEQ ID NO: 7 (SNWKWWPGIFD), known in the art as “QBP1,” is a peptide shown to target polyQ repeats on Htt as well as reduce and delay aggregation of Htt. Moreover, QBP1, when integrated into a protein transduction domain delivery system, has been shown to penetrate cells and reach the striatum. These characteristics are discussed in detail in Popiel, H. A. et al. Protein Transduction Domain-Mediated Delivery of QBP1 Suppresses Polyglutamine-Induced Neurodegeneration in Vivo. Mol. Ther. 2007, 15 (2), 303-309, and Popiel, H. A. et al. Delivery of the Aggregate Inhibitor Peptide QBP1 into the Mouse Brain Using PTDs and Its Therapeutic Effect on Polyglutamine Disease Mice. Neurosci. Lett. 2009, 449 (2), 87-92, each of which is incorporated by reference in its entirety, and more specifically to facilitate the understanding of QBP1, to the extent not inconsistent with the description herein. Given the targeting properties of the PLP platform to specifically target intracellular proteins in the central nervous system, it believed the incorporation of SEQ ID NO: 7 in the co-PLP may improve therapeutic payload in neuronal cells.

SEQ ID NO: 8 (GRKKRRQRRRPPQSSEIVLDGTDN), known in the art as “ED11,” is a synthetic peptide based on the Htt Caspase-6 cleavage site. ED11 was designed to target mtHtt/caspase 6 inhibition to prevent mtHtt cleavage. SEQ ID NO: 8 is an example of a mechanism different from the VCP/mtHtt inhibition mechanism contemplated for SEQ ID NOs: 1-5. Accordingly, it is believed a co-PLP incorporating both SEQ ID NO: 8 and any of SEQ ID NOs: 1-5 as peptide side chains may results in an effective dual therapy of HD. The properties of ED11 are discussed in detail in Aharony, et al. A Huntingtin-Based Peptide Inhibitor of Caspase-6 Provides Protection from Mutant Huntingtin-Induced Motor and Behavioral Deficits. Hum. Mol. Genet. 2015, 24 (9), 2604-2614, which is incorporated by reference in its entirety, and more specifically to facilitate understanding of ED11, to the extent not inconsistent with the description herein.

For each of the peptides disclosed in this Example 11, it will be appreciated that each peptide may be modified, synthesized, and polymerized according to procedures discussed in Example 1. For example, arginine or lysine residues may be added to any of SEQ ID NOs: 6-8 to introduce a positive charge and improve cellular uptake prior to conjugation to an unsaturated monomer, such as N-(hexanoic acid)-exo-5-norbornene-2,3-dicarboximide, and polymerization. Furthermore, co-PLPs incorporating any of SEQ ID NOs: 6-8 will be tested for cell viability, cell uptake, mitochondrial localization, mitochondrial fragmentation prevention, BBB permeation, and stability pursuant to methods discussed in Examples 2, 3, 5, and 6. To confirm suitability for in vivo treatments, immunocompatibility, hemocompatibility, pharmacokinetic profiling, toxicity, and efficacy studies will be conducted according to procedures discussed in Examples 7-10.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, including any isomers, enantiomers, and diastereomers of the group members, are disclosed separately. When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individual or in any combination. Additionally, unless otherwise specified, all isotopic variants of compounds disclosed herein are intended to be encompassed by the disclosure. For example, it will be understood that any one or more hydrogens in a molecule disclosed can be replaced with deuterium or tritium. Isotopic variants of a molecule are generally useful as standards in assays for the molecule and in chemical and biological research related to the molecule or its use. Methods for making such isotopic variants are known in the art. Specific names of compounds are intended to be exemplary, as it is known that one of ordinary skill in the art can name the same compounds differently.

Certain molecules disclosed herein may contain one or more ionizable groups [groups from which a proton can be removed (e.g., —COOH) or added (e.g., amines) or which can be quaternized (e.g., amines)]. All possible ionic forms of such molecules and salts thereof are intended to be included individually in the disclosure herein. With regard to salts of the compounds herein, one of ordinary skill in the art can select from among a wide variety of available counterions those that are appropriate for preparation of salts of this invention for a given application. In specific applications, the selection of a given anion or cation for preparation of a salt may result in increased or decreased solubility of that salt.

Every device, system, formulation, combination of components, or method described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

One of ordinary skill in the art will appreciate that starting materials, biological materials, reagents, synthetic methods, purification methods, analytical methods, assay methods, and biological methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A polymer comprising:
a first repeating unit comprising a first polymer backbone subunit directly or indirectly covalently linked to a first functional sidechain comprising a peptide having 75% or greater sequence identity of a homologous region between VCP and Htt.

2-3. (canceled)

4. A polymer characterized by a formula (FX1): 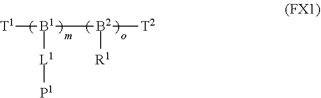
wherein:
each P¹ independently comprises a peptide;
at least one P¹ independently, or in combination with other instances of P¹, comprises a HV3 peptide or a modified HV3 peptide;
T¹ and T² are each independently polymer backbone terminating groups that can be the same or different;
B¹ and B² are each independently a polymer backbone subunit;
L¹ is optionally present and is a linking group;
R¹ is independently a substituent;
m is an integer from 2 to 1000;
o is an integer from 0 to 1000;
each connecting line in the formula (FX1) represents a covalent linkage comprising at least one of a single bond, a double bond, one or more atoms, or any combination thereof, optionally wherein the one or more atoms comprise carbon, nitrogen, and/or oxygen atoms;
each instance of B¹, B², L¹, R¹, and P¹ is the same as or different from any other instance of B¹, B², L¹, R¹, and P¹, respectively; and
when o is an integer from 1 to 1000 and/or at least one instance of P¹ is different from another instance of P¹, the polymer is a block copolymer or a statistical copolymer.

5. The polymer of claim 4, wherein the polymer is characterized by a formula (FX2): 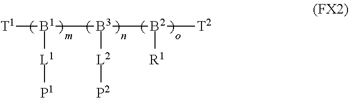
wherein:
T¹ and T² are each independently polymer backbone terminating groups that can be the same or different;
B¹, B², and B³ are each independently polymer backbone subunits;
each L¹ and L² is optionally present and each is independently a linking group;
each P¹ and P² independently comprise a peptide or a small molecule;
at least one P¹ independently, or in combination with other instances of P¹, comprises a HV3 peptide or a modified HV3 peptide;
each instance of P² is different from each instance of P¹;
each R¹ is independently a substituent;
m is an integer selected from the range of 2 to 1000;
n is an integer selected from the range of 1 to 1000;
o is an integer selected from the range of 0 to 1000;
each connecting line in formula (FX2) represents a covalent linkage comprising at least one of a single bond, a double bond, one or more atoms, or any combination thereof, optionally, for example, each connecting line represents a single bond or double bond;
each instance of B¹, B², B³, L¹, L², R¹, P¹, and P² is the same as or different from any other instance of B¹, B², B³, L¹, L², R¹, P¹, and P², respectively; and
the polymer is a block copolymer or a statistical copolymer.

6. The polymer of claim 4, wherein at least one of B¹ or B² comprises a polymerized monomer comprising an unsaturated monomer, wherein the unsaturated monomer comprises an ethylenically unsaturated monomer, a norbornene monomer, or a norbornene dicarboxyimide.

7.-9. (canceled)

10. The polymer of claim 4, wherein each instance of L¹ is independently selected from a single bond, an oxygen, and groups having an alkylene group, a heteroalkylene group, an alkenylene group, an arylene group, an alkoxy group, an acyl group, a triazole group, a diazole group, a pyrazole group, and combinations thereof.

11.-12. (canceled)

13. The polymer of claim 4, wherein each instance of R¹, T¹, and T² independently is hydrogen, C₁-C₃₀ alkyl, C₃-C₃₀ cycloalkyl, C₅-C₃₀ aryl, C₅-C₃₀ heteroaryl, C₁-C₃₀ acyl, C₁-C₃₀ hydroxyl, C₁-C₃₀ alkoxy, C₂-C₃₀ alkenyl, C₂-C₃₀ alkynyl, C₅-C₃₀ alkylaryl, —CO₂R⁴, —CONR⁵R⁶, —COR⁷, —SOR⁸, —OSR⁹, —SO₂R¹⁰, —OR¹¹, —SR¹², —NR¹³R¹⁴, —NR¹⁵COR¹⁶, C₁-C₃₀ alkyl halide, phosphonate, phosphonic acid, silane, siloxane, silsesquioxane, C₂-C₃₀ halocarbon chain, C₂-C₃₀ perfluorocarbon, C₂-C₃₀ polyethylene glycol, a metal, or a metal complex, wherein each of R⁴-R¹⁶ independently is H, C₅-C₁₀ aryl, or C₁-C₁₀ alkyl.

14.-21. (canceled)

22. The polymer of claim 1, wherein the polymer is a high-density brush polymer characterized by a brush density greater than or equal to 75%.

23. (canceled)

24. The polymer of claim 1, wherein the polymer is metaphilic.

25. (canceled)

26. The polymer of claim 4, wherein the polymer is characterized by a P¹ peptide density of greater than or equal to 85%.

27.-28. (canceled)

29. The polymer of claim 4, wherein at least 75% of all instances of P¹ on a number basis comprises an amino acid sequence derived from Htt.

30. The polymer of claim 4, wherein at least 75% of all instances of P¹ on a number basis comprises a sequence identity having 75% or greater sequence identity of an HV3 peptide or a modified HV3 peptide.

31. The polymer of claim 4, wherein at least one P¹ comprises a protein-protein interaction inhibitor, and wherein the protein-protein interaction comprises the interaction of VCP with mtHtt.

32. (canceled)

33. The polymer of claim 5, wherein at least one of P¹ or P² is characterized by a net positive charge.

34. The polymer of claim 5, wherein at least one of P¹ or P² further comprises a charge modulating domain.

35.-37. (canceled)

38. The polymer of claim 5, wherein the polymer has a P¹:P² ratio of between 1:1 and 15:1.

39. (canceled)

40. The polymer of claim 4, wherein at least one P¹ comprises a sequence having 75% or greater sequence identity of:
SEQ ID NO: 1
(HVLVMSATRR);
SEQ ID NO: 2
(HVLVMSATKK);
SEQ ID NO: 3
(HVLVMSATRRRR);
or
SEQ ID NO: 4
(HVLVMSATKKKK).

41.-47. (canceled)

48. The polymer of claim 4, wherein at least 75% of all instances of P¹ on a number basis comprise:
SEQ ID NO: 1
(HVLVMSATRR);
SEQ ID NO: 2
(HVLVMSATKK);
SEQ ID NO: 3
(HVLVMSATRRRR);
SEQ ID NO: 4
(HVLVMSATKKKK);
SEQ ID NO: 9
(HVLVMSAT);
or
any combination thereof.

49.-50. (canceled)

51. The polymer of claim 5, wherein at least one P² comprises a sequence having 75% or greater sequence identity of:
SEQ ID NO: 6
(AASSGVSTPGSAGHDIITEQPRS);
SEQ ID NO: 7
(SNWKWWPGIFD);
or
SEQ ID NO: 8
(GRKKRRQRRRPPQSSEIVLDGTDN).

52.-53. (canceled)

54. The polymer of claim 4, wherein at least 85% of all instances of P¹ on a number basis do not comprise sequences having cysteine residues.

55. (canceled)

56. The polymer of claim 5, wherein at least one of P¹ or P² comprises or further comprises a point mutation or substitution to comprise at least one serine.

57. A composition comprising the polymer of claim 1 and a pharmaceutically acceptable excipient.

58. A method for selectively inhibiting the mitochondrial autophagy pathway in a cell, the method comprising:
contacting the cell with an effective amount of the polymer of claim 1;
wherein the contacting results in the selective inhibition of the mitochondrial autophagy pathway in the cell.

59. A method for selectively inhibiting the interaction between VCP and mtHtt in a cell, the method comprising:
contacting the cell with an effective amount of the polymer of claim 1;
wherein the contacting results in the selective inhibition of the interaction between VCP and mtHtt in the cell.

60. (canceled)

61. A method for preventing or treating a neurodegenerative disease or condition in a subject, the method comprising:
administering to the subject a therapeutically effective amount of the polymer of claim 1;
thereby preventing or treating the neurodegenerative disease or condition in the subject.

62.-63. (canceled)

64. The method of claim 61, wherein the neurodegenerative disease or condition comprises Huntington's disease.

65. (canceled)

66. A method of targeting a mitochondrial localized protein in a cell comprising:
introducing the polymer of claim 1;
wherein the introducing results in at least a portion of the polymer to bind with at least a portion of the mitochondrial localized protein;
thereby targeting a mitochondrial localized protein in a cell.

67.-69. (canceled)

70. A therapeutic peptide having 75% or greater sequence identity of SEQ ID NO: 1, SEQ ID NO: 2, SEQ ID NO: 3, or SEQ ID NO: 4.

71.-77. (canceled)
